(19) United States
(12) Patent Application Publication
(10) Pub. No.: US 2006/0234223 A1
(43) Pub. Date: Oct. 19, 2006

(54) Association of snps in the comt locus and neighboring loci with schizophrenia, bipolar disorder, breast cancer and colorectal cancer

(76) Inventors: Ariel Darvasi, Zion (IL); Naomi Zak, Jerusalem (IL)

Correspondence Address: Martin Moynihan;Anthony Castorina, Suite 207, 2001 Jefferson Davis Highway, Arlington, VA, 22202, US

(21) Appl. No.: 10/504,146

(22) PCT Filed:
(86) PCT No.: PCT/IL03/00140

Related U.S. Application Data
(60) Provisional application No. 60/357,822, filed on Feb. 21, 2002.
(60) Provisional application No. 60/437,459, filed on Jan. 2, 2003.

(51) Int. Cl. C12Q 1/68

(52) U.S. Cl. 435/6

(57) ABSTRACT

Methods and kits used for determining predisposition and/or diagnosis of schizophrenia, bipolar disorder, breast cancer and colorectal cancer using genotypes in the COMT locus are disclosed. Also disclosed are methods and drugs for treating these disorders. Further disclosed are methods and kits useful for prediction drug responsiveness towards mental disorders drugs, and more specifically towards schizophrenia drugs.

### FIELD AND BACKGROUND OF THE INVENTION

The present invention relates to (i) the use of genetic markers, such as single nucleotide polymorphisms (SNPs) for the identification of genes related to and for determining a risk of developing complex (e.g., multigenic) diseases; (ii) methods of identifying novel drugs for the treatment and/or prevention of complex diseases; (iii) methods of treating and/or preventing complex diseases; and (iv) pharmacogenomic methods of determining drug responsiveness. More particularly, the present invention relates to (i) the use of SNPs in the COMT locus and neighboring loci for the identification of genes related to, and for determining a risk of developing schizophrenia, bipolar disorder, breast cancer and colorectal cancer; (ii) methods of identifying novel drugs for the treatment and/or prevention of schizophrenia, bipolar disorder, breast cancer and colorectal cancer; (iii) methods of treating and/or preventing schizophrenia, bipolar disorder, breast cancer and colorectal cancer; and (iv) pharmacogenomic methods of determining drug responsiveness in mental disorders, schizophrenia in particular.

Common and Complex Disorders Have Genetic Components

Recent advances in the fields of genetics and molecular biology have allowed identification of forms, or alleles, of human genes that lead to diseases. Most of the genetic variations responsible for human diseases identified so far belong to the class of single gene disorders which are also referred to in the art as Mendelian disorders. As this name implies, the development of single gene disorders is determined, or largely influenced, by the alleles of a single gene. The alleles that cause single gene disorders are, in general, highly deleterious (and highly penetrant) to individuals who carry them. Therefore, these alleles and their associated diseases, with some exceptions, tend to be very rare in the human population.

Common diseases such as certain central nervous system (CNS) disorders, various cancers and heart diseases often aggregate in families, suggesting that there is a genetic component to the disease. For example, breast cancer is more common among first-degree relatives of breast cancer patients than it is in the general population. In addition, alcoholism or certain forms of mental illness can result from genetic predisposition demonstrated by the higher risk of the children of alcoholics or mentally ill individuals to develop the same illness, even when they are adopted away from their biological parents early in infancy, so as to preclude environmental effects. Evidence of this sort is available for many common diseases. However, complex traits which include drug efficacy, response and tolerance to toxicity are often due to the combined action of multiple genes, as well as environmental factors. The genetics of complex diseases is therefore more complicated than that of Mendelian diseases since predisposing genes do not produce the disease in every person who carries them.

Central Nervous System (CNS) Disorders

Central nervous system (CNS) disorders have complex and poorly understood etiologies with overlapping symptoms that are difficult to quantitate. The development of treatment regimes and drugs for CNS disorders requires the understanding of the multigenic causes of the various CNS disorders. CNS disorders include mental retardation syndromes which appear early in life, psychiatric diseases such as schizophrenia and bipolar disorder which appear later during adolescence and early adulthood and late-onset degenerative disorders such as, for example, Parkinson's and Alzheimer's diseases.

Schizophrenia

Schizophrenia is one of the most severe and debilitating of psychiatric disorders. Schizophrenia is used to describe a cluster of symptoms that typically includes delusions, hallucinations, disordered thinking and emotional unresponsiveness. Its onset is usually in late adolescence or early adult life and often it becomes chronic and disabling. Men and women are at equal risk of developing this illness; however, most males become ill between 16 and 25 years of age, while females develop symptoms between 25 and 30 years of age. Individuals with schizophrenia often experience both “positive” symptoms (e.g., delusions, hallucinations, disorganized thinking, and agitation) and “negative” symptoms (e.g., lack of drive or initiative, social withdrawal, apathy, and emotional unresponsiveness).

Schizophrenia affects 1% of the world population. There are an estimated 45 million people with schizophrenia world wide, with more than 33 million of them in the developing countries.

Moreover, schizophrenia accounts for one fourth of all mental health costs and takes up one in three psychiatric hospital beds. Most schizophrenia patients will never be able to provide for themselves. The cost of schizophrenia to society is enormous. In the United States, for example, the direct cost of treatment of schizophrenia has been estimated to be close to 0.5% of the gross national product. Standardized mortality ratios (SMRs) for schizophrenic patients are estimated to be two to four times higher than for the general population, and their overall life expectancy is 20% shorter than of the general population. The most common cause of death among schizophrenic patients is suicide (in 10% of patients) which represents a 20 times higher risk than for the general population. Deaths from heart disease or from diseases of the respiratory and digestive system are also increased among schizophrenic patients.

Bipolar Disorder

Bipolar affective disorder is a relatively common disorder affecting about 1.3% of the population and have been reported to constitute about half of the mood disorders seen in a psychiatric clinic. In addition to the severe effects on patients' social development, suicide completion rates among bipolar patients are reported to be about 15%.

Bipolar disorder is a characterized by phases of excitement and phases of depression; the excitement phases are oftentimes referred to as mania or hypomania. The depression can alternate or occur in various admixtures with the mania and at different degrees of severity and with varying time periods. Because bipolar disorder can exist in different forms and display different symptoms, the classification of bipolar disorder has been the subject of extensive study resulting in the definition of bipolar disorder subtypes and widening of the overall concept to include patients previously thought to be suffering from other disorders. Patients suffering from bipolar disorder often share certain clinical signs, symptoms, responsiveness to various treatments and neurobiological features with those suffering from other with psychotic illnesses, and therefore present a diagnostic challenge to the psychiatrist. Nontheless, because the course and etiology of bipolar disorder and various mood and psychotic disorders can differ greatly, it is critical to characterize the illness as early as possible in order to offer means to manage the illness over a long term.

Similar to schizophrenia, the cost of bipolar disorder to society is enormous. The mania associated with the disease impairs performance, causes psychosis, and often results in hospitalization. Furthermore, the earlier the onset, the more severe are the effects of interrupted education and social development and abilities.

The Diagnostic and Statistical Manual of Mental Disorders (DSM-IV) classification of bipolar disorder distinguishes among four types of disorders based on the degree and duration of mania or hypomania. Mania is recognized by elevated, expansive or irritable mood as well as by distractive, impulsive behavior, increased activity, grandiosity, elation, racing thoughts, and pressured speech. The DSM-IV classification is as follows: Bipolar disorder I, including patients displaying mania for at least one week; bipolar disorder II, including patients displaying hypomania for at least 4 days, characterized by milder symptoms of excitement than mania, who have not previously displayed mania, and have previously suffered from episodes of major depression; bipolar disorder not otherwise specified (NOS), including patients otherwise displaying features of bipolar disorder II but not meeting the 4 day duration for the excitement phase, or who display hypomania without an episode of major depression; and cyclothymia, including patients who show numerous manic and depressive symptoms that do not meet the criteria for hypomania or major depression, but which display symptoms for over two years without a symptom-free interval of more than two months.

Bipolar disorder has been found to vary with gender depending on the type of disorder; for example, bipolar disorder I is found equally among men and women, while bipolar disorder II is reportedly more common in women. The age of onset of bipolar disorder is typically in the teenage years and diagnosis is typically made in the patient's early twenties. Bipolar disorder also occurs among the elderly, generally as a result of a medical or neurological disorder.

Diagnosis of bipolar disorder can be very challenging. One particularly troublesome difficulty is that some patients exhibit mixed states, simultaneously manic and dysphoric or depressive, but do not fall into the DSM-IV classification because not all required criteria for mania and major depression are met daily for at least one week. Other difficulties include classification of patients in the DSM-IV groups based on duration of phase since patients often cycle between excited and depressive episodes at different rates. In particular, it is reported that the use of antidepressants may alter the course of the disease for the worse by causing “rapid-cycling”. In addition, psychiatrists must distinguish between agitated depression and mixed mania; it is common that patients with major depression (14 days or more) exhibit agitation, resulting in bipolar-like features. A yet further complicating factor is that bipolar patients have an exceptionally high rate of substance, particularly alcohol, abuse. While the prevalence of mania in alcoholic patients is low, it is well known that substance abusers can show excited symptoms. There is thus a need to develop useful methods for accurate diagnosis of bipolar disorder.

Treatment for Schizophrenia and Bipolar Disorder

As there are currently no cures for bipolar disorder or schizophrenia, the objective of treatment is to reduce the severity of the symptoms, if possible to the point of remission. Due to overlap in symptoms, schizophrenia and bipolar disorder are often treated with some of the same medicaments including antipsychotics and neuroleptics drugs.

In schizophrenia, for example, antipsychotic medications are the most common and most valuable treatments. There are four main classes of antipsychotic drugs which are commonly prescribed for schizophrenia. The first, neuroleptics, exemplified by chlorpromazine (Thorazine™), has revolutionized the treatment of schizophrenic patients by reducing positive (psychotic) symptoms and preventing their recurrence. Patients receiving chlorpromazine have been able to leave mental hospitals and live in community programs or their own homes. However, these drugs are far from ideal. Some 20% to 30% of patients do not respond to them at all, and others eventually relapse. These drugs produce serious neurological side effects, including rigidity and tremors in the arms and legs, muscle spasms, abnormal body movements, and akathisia (restless pacing and fidgeting). These side effects are so troublesome that many patients simply refuse to take the drugs. Furthermore, neuroleptics do not improve the so-called negative symptoms of schizophrenia and the side effects may even exacerbate these symptoms. Thus, despite the clear beneficial effects of neuroleptics, even some patients who have a good short-term response will ultimately deteriorate in overall functioning.

The well-known deficiencies in the standard neuroleptics have stimulated a search for new treatments and have led to a new class of drugs termed “atypical neuroleptics”. The first atypical neuroleptic, clozapine, is effective for about one third of patients who do not respond to standard neuroleptics. It seems to reduce negative as well as positive symptoms, or at least exacerbates negative symptoms less than standard neuroleptics do. Moreover, it has beneficial effects on overall functioning and may reduce the chance of suicide in schizophrenic patients. It does not produce the troubling neurological symptoms of the standard neuroleptics, or raise blood levels of the hormone prolactin, excess of which may cause menstrual irregularities and infertility in women, impotence or breast enlargement in men. Many patients who cannot tolerate standard neuroleptics have been able to take clozapine. However, clozapine has serious limitations. It was originally withdrawn from the drug market because it can cause agranulocytosis, a potentially lethal inability to produce white blood cells. Agranulocytosis remains a threat that requires careful monitoring and periodic blood tests. Clozapine can also cause seizures and other disturbing side effects (e.g., drowsiness, lowered blood pressure, drooling, bed-wetting, and weight gain). Thus, it is usually taken only by patients who do not respond to other drugs.

Researchers have developed a third class of antipsychotic drugs that have the virtues of clozapine without its defects. One of these drugs is risperidone (Risperdal™). Early studies suggest that it is as effective as standard neuroleptic drugs for positive symptoms and may be somewhat more effective for negative symptoms. It produces more neurological side effects than clozapine but fewer than standard neuroleptics. However, it raises prolactin levels. Risperidone is now prescribed for a broad range of psychotic patients, and many clinicians seem to use it before clozapine for patients who do not respond to standard drugs, because they regard it as being safer. Another new drug is olanzapine (Zyprexa™), which is at least as effective as standard drugs for positive symptoms and more effective for negative symptoms. It has few neurological side effects at ordinary clinical doses, and it does not significantly raise prolactin levels. Although it does not produce most of clozapine's most troubling side effects, including agranulocytosis, some patients taking olanzapine may become sedated or dizzy, develop dry mouth, or gain weight. In rare cases, liver function tests become transiently abnormal.

However, the use of drugs is often delayed for patients experiencing a first episode of the illness. The patients may not realize that they are ill, or they may be afraid to seek help; family members sometimes hope the problem will simply disappear or cannot persuade the patient to seek treatment; clinicians may hesitate to prescribe antipsychotic medications when the diagnosis is uncertain because of potential side effects. Indeed, at the first manifestation of the disease, schizophrenia is difficult to distinguish from bipolar disorder, severe depression, drug-related disorders, and stress-related disorders.

Moreover, all the known treatments for schizophrenia and bipolar disorder act against the symptoms of the disease. There is thus a strong need for new molecules directed against targets which are involved in the causal mechanisms of schizophrenia and bipolar disorder. Therefore, tools facilitating the discovery and characterization of these targets are necessary and useful.

Schizophrenia and bipolar disorder are considered brain diseases, and as such are subjected to neuroimaging and neuropathological studies. These studies demonstrate pathological changes in the brain which probably occurred early in brain development [Engelien, A., Stern, E., Silbersweig, D. (2001). Functional neuroimaging of human central auditory processing in normal subjects and patients with neurological and neuropsychiatric disorders. J. Clin. Exp. Neuropsychol. 23: 94-120]. In addition, the previously known dopamine hypothesis, i.e., that depression is caused by deficiency in dopamine, norepinephrine and/or serotonin has been extensively revised and is no longer considered as a primary causative model.

The aggregation of schizophrenia and bipolar disorder in families, the evidence from twin and adoption studies, and the lack of variation in worldwide incidence, indicate that schizophrenia and bipolar disorder are primarily genetic conditions. For example, although the general risk for having schizophrenia is 1%, the risk for schizophrenia increases to 3% if one grandparent is affected, to 10% if one parent is affected, and to approximately 40% if both parents are affected.

Consequently, there is a strong need to identify genes involved in schizophrenia and bipolar disorder. The knowledge of these genes will allow researchers to understand the etiology of schizophrenia and bipolar disorder and could lead to the development of drugs and medications directed against the cause of the diseases, not just against their symptoms.

There is also a great need for new methods of detecting susceptibility to schizophrenia and bipolar disorder, as well as for preventing or following up the development of the diseases. Diagnostic tools could also prove extremely useful. Indeed, early identification of subjects at risk of developing schizophrenia would enable early and/or prophylactic treatment to be administered.

Knowledge of genetic variations in the causative genes will also enable the development of pharmacogenomics in which the treatment is genotype-specific.

Cancer Disease

Cancer is characterized primarily by an increase in the number of abnormal cells derived from a given normal tissue, invasion of adjacent tissues by these abnormal cells, and lymphatic or blood-borne spread of malignant cells to regional lymph nodes and to distant sites (metastasis).

The genetics of cancer is complicated, involving multiple dominant, positive regulators of the transformed state (e.g., oncogenes) as well as multiple recessive, negative regulators (e.g., tumor suppressor genes). Over one hundred oncogenes and several tumor suppressor genes have thus far been characterized. The involvement of so many genes underscores the complexity of the growth control mechanisms that operate in the cells to maintain the integrity of a normal tissue. However, no single gene has been shown to participate in the development of all, or even the majority of human cancers. The most common oncogene mutations are in the H-ras gene, found in 10-15% of all solid tumors [Anderson, J. A., Irish, J. C., Ngan, B. Y. (1992). Prevalence of RAS oncogene mutation in head and neck carcinomas. J. Otolaryngol. 21: 321-6]. Several tumor suppressor genes which confer susceptibility to various cancers have been cloned. These include the genes for retinoblastoma (RB 1), Wilms' tumor (WT1), Li-Fraumeni (p53), familial adenomatous polyposis (APC), neurofibromatosis type 1 (NF1), neurofibromatosis type 2 (NF2), von Hippel-Lindau syndrome (VHL), multiple endocrine neoplasia type 2A (MEN2A) and melanoma (CDKN2). Other tumor suppressor loci include the genes for multiple endocrine neoplasia type 1 (MEN1), Lynch cancer family syndrome 2 (LCFS2), neuroblastoma (NB), basal cell nevus syndrome (BCNS), Beckwith-Wiedemann syndrome (BWS), renal cell carcinoma (RCC), tuberous sclerosis 1 (TSC1) and tuberous sclerosis 2 (TSC2). The tumor suppressor genes that have been characterized to date encode products with similarities to a variety of protein types, including DNA binding proteins (WT1), ancillary transcription regulators (RB1), GTPase activating proteins or GAPs (NF1), cytoskeletal components (NF2), membrane bound receptor kinases (MEN2A), cell cycle regulators (CDKN2) and others with no obvious similarity to known proteins (APC and VHL). In many cases, tumor suppressor genes originally identified through genetic studies were shown to be lost or mutated in sporadic tumors. The deletions are often referred to as “loss of heterozygosity” (LOH) i.e., loss of a single allele, but tumors may also involve homozygous deletion of both alleles. In cases of LOH, the remaining allele is presumed to be nonfunctional, either because of a pre-existing inherited mutation, or because of a secondary sporadic mutation.

Breast Cancer

Breast cancer is the most common form of malignancy in women. One in eight to nine women in the Western world will develop breast cancer in their lifetime [Grover, P. L. and F. L. Martin (2002). The initiation of breast and prostate cancer. Carcinogenesis 23(7): 1095-102] and about 25% of them will ultimately die from the disease. For example, 180,000 American women were diagnosed with breast cancer in 1997 and 43,900 succumbed to the disease [Parker, S. L. et al., (1997). Cancer Statistics, 1997. CA Cancer J. Clin. 47: 5-27]. A disturbing fact is the observation that breast cancer has been increasing at a rate of 3 percent per year since 1980 [Niederhuber, J. E., ed. Current Therapy in Oncology, B. C. Decker, Mosby, 1993].

Breast cancer is among the more slow-growing tumors, and the preclinical period before diagnosis and the clinical phases after initial treatment and even after the appearance of metastasis are measured in years and decades. Nevertheless, some patients have aggressive forms of the disease and have a worse prognosis. During the long clinical phase there is ample opportunity for clonal mutation and evolution, and it seems probable that individual patients may have multiple tumor clones, each with its own growth rate, propensity to metastasize, and sensitivity to drugs.

In both Europe and North America, early detection campaigns based on mass screening programs have been introduced in an effort to reduce mortality rates. Widespread use of these procedures resulted in an increased frequency of detection of breast cancer, which in turn has contributed to a greater number of women with early stage disease [Harris et al., 1993, Cancer: Principles and Practice of Oncology, eds. De Vita, V. T., Hellman, S., & Rosenberg, S. A. (J. B. Lippincott, Philadelphia), 4th Ed., pp. 1264-1332]. Given the high degree of morphological heterogeneity of most breast cancers, it is at present still difficult to assess appropriate therapy and risk of recurrence for the majority of women who present with early stage disease. The currently available criteria affecting prognosis are tumor size and grade, lymph node status, DNA ploidy and mitotic index, lymphovascular invasion, as well as estrogen receptor status [Harris et al., 1993, Cancer: Principles and Practice of Oncology, eds. De Vita, V. T., Hellman, S., & Rosenberg, S. A., J. B. Lippincott, Philadelphia, 4th Ed., pp. 1264-1332]. These multiple parameters remain poorly correlated with the molecular events associated with a multi-step progression of malignancy.

Genetic factors contribute to 10-15% of all breast cancer cases. Breast cancer has been subdivided into two types, early-age onset and late-age onset, based on an inflection in the age-specific incidence curve around age 50. The proportion of breast cancer cases predicted to be attributable to breast cancer susceptibility gene(s) decreases markedly with age; approximately 33% of cases age 20-29 years compared with approximately 2% of cases age 70-79 years [Claus, E. B., Schildkraut, J. M., Thompson, W. D., Risch, N. J. (1996). The genetic attributable risk of breast and ovarian cancer. Cancer 77: 2318-24]. Mutations in BRCA1 are estimated to account for 52% of familial breast cancer and to 81% of families with both breast and ovarian cancer. In one large-scale study it was found that mutations in BRCA2 account for 32% families with breast cancer and to 14% of breast-ovarian cancer families. Conversely, the majority of families with male and female breast cancer were due to BRCA2 (76%) [Ford D et al., (1998). Genetic heterogeneity and penetrance analysis of the BRCA1 and BRCA2 genes in breast cancer families. The Breast Cancer Linkage Consortium. Am. J. Hum. Genet. 62: 676-89]. Breast cancer in the remaining families was not linked to BRCA1 or BRCA2, suggesting the involvement of other predisposition genes in the etiology of breast cancer.

It has been suggested that heterozygote carriers for defective forms of the Ataxia-Telangectasia gene (ATM) are at higher risk for breast cancer. In addition, loss of heterozygosity in the region of the ATM gene on chromosome 11 was found in about 40% of sporadic breast tumors. However, screening for ATM mutations in sporadic breast cancer cases has not revealed the magnitude of involvement of the ATM gene expected [Angele S, Hall J. (2000). The ATM gene and breast cancer: is it really a risk factor? Mutat. Res. 462: 167-78]. On the other hand, factors affecting exposure to endogenous hormones might be involved in increased risk for breast cancer. Thus, genes involved in the metabolism of sex hormones are strong candidates for breast cancer susceptibility genes. Among them are the CYP 17, CYP 19 and the 17β-hydroxysteroid dehydrogenase type 2 genes. Several case-control association studies revealed statistically significant differences in genotype frequencies of several polymorphisms, including the CYP19 (TTTA)ₙ polymorphism, the GSTP1 Ile105Val polymorphism and the p53 Arg72Pro polymorphism. In addition, the GSTM1 gene deletion was found to be significantly associated with postmenopausal breast cancer [reviewed in Dunning, A. M., Healey, C. S., Pharoah, P. D. P., Teare, MD., Ponder, B. A. J., Easton, D. F. (1999). A systematic review of genetic polymorphisms and breast cancer risk. Cancer Epidemiology, Biomarkers & Prevention, 8: 843-854]. However, precise estimation of the risks associated with these genes will require much larger studies than those reported.

Colorectal Cancer

Colorectal cancer is the second most common cause of cancer death in the United States and the third most prevalent cancer in both men and women with approx. 160,000 new cases of colon cancer per year [M. L. Davila & A. D. Davila, Screening for Colon and Rectal Cancer, in Colon and Rectal Cancer 47 (Peter S. Edelstein ed., 2000)]. The lifetime risk for colorectal cancer is 5%, making it an important public health issue [Calvert, P. M. and H. Frucht (2002). The genetics of colorectal cancer. Ann Intern Med 137(7): 603-12]. Cancer of the gastrointestinal tract, especially colon cancer, is a highly treatable and often a curable disease when localized to the bowel. Surgery is the primary treatment and results in cure in approximately 50% of patients. Recurrence following surgery is a major problem and often is the ultimate cause of death. The prognosis of colon cancer is clearly related to the degree of penetration of the tumor through the bowel wall and the presence or absence of nodal involvement. These two characteristics form the basis for all staging systems developed for this disease. Bowel obstruction and bowel perforation are indicators of poor prognosis. Elevated pretreatment serum levels of carcino-embryonic antigen (CEA) and carbohydrate antigen 19-9 (CA 19-9) also have negative prognostic significance.

Nearly all cases of colorectal cancer arise from adenomatous polyps, some of which mature into large polyps, undergo abnormal growth and development, and ultimately progress into cancer. This progression would appear to take at least 10 years in most patients, rendering it a readily treatable form of cancer if diagnosed early, when the cancer is localized. Although the understanding of the etiology of colon cancer is undergoing continual refinement, extensive research in this area points to a combination of factors, including age, hereditary and non-hereditary conditions, and environmental/dietary factors. Age is a key risk factor in the development of colorectal cancer, with men and women over 40 years of age become increasingly susceptible to the disease. Incidence rates increase considerably in each subsequent decade of life. A number of hereditary and nonhereditary conditions have also been linked to a heightened risk of developing colorectal cancer, including familial adenomatous polyposis (FAP), hereditary nonpolyposis colorectal cancer (Lynch syndrome or HNPCC), a personal and/or family history of colorectal cancer or adenomatous polyps, inflammatory bowel disease, diabetes mellitus, and obesity [Henry T. Lynch & Jane F. Lynch, Hereditary 5 Nonpolyposis Colorectal Cancer (Lynch Syndromes), in Colon and Rectal Cancer 67-68 (Peter S. Edelstein ed., 2000)]. In the case of FAP, the tumor suppressor gene APC (adenomatous polyposis coli), located at 5q21, has been either mutationally inactivated or deleted [Alberts et al., Molecular Biology of the Cell 1288 (3d ed. 1994)]. The APC protein plays a role in a number of functions, including cell adhesion, apoptosis, and repression of the c-myc oncogene [N. R. Hall & R. D. Madoff, Genetics and the Polyp-Cancer Sequence, Colon and Rectal Cancer 8 (Peter S. Edelstein, ed., 2000)]. Of those patients with colorectal cancer who have normal APC genes, over 65% have such mutations in the cancer cells but not in other tissues. In the case of HPNCC, patients manifest abnormalities in the tumor suppressor gene HNPCC, but only about 15% of tumors contain the mutated gene. Other genes have also been implicated in colorectal cancer, including the K-ras, N-ras, H-ras and c-myc oncogenes, the tumor suppressor genes DCC (deleted in colon carcinoma) and p53. Conversely, environmental/dietary factors associated with a reduced risk of colorectal cancer include a diet high in fiber, folic acid, calcium, and hormone-replacement therapy in post-menopausal women.

Identification of breast cancer and colorectal cancer susceptibility loci would permit the early detection of at-risk individuals and greatly increase the ability to understand the initial steps that lead to cancer development. As susceptibility loci are often altered during tumor progression, cloning these genes could also be important in the development of better diagnostic and prognostic tools, as well as better cancer therapies.

Genetic Strategies for the Identification of Disease-Causing Genes

The traditional genetic strategy for the identification of disease-causing genes is based on linkage analysis studies of large informative families, which are studied using microsatellite markers (e.g., di-, tri-, or tetra-nucleotide repeats). Genetic linkage analysis calculates the probabilities of recombination between the disease-causing gene and the chromosomal markers used, according to the genealogical tree, the transmission of the disease and the transmission of the markers. Thus, if a particular allele of a given marker is transmitted with the disease more often than expected by chance (recombination level between 0 and 0.5), it is possible to deduce that the target gene in question is found in the neighborhood of the marker. However, the families included in the study must satisfy the “informativeness” criteria: several affected subjects (whose constitutional DNA is available) per generation, and at best a large number of siblings.

Linkage analysis has been successfully applied to map simple genetic traits that show clear Mendelian inheritance patterns and which have a high penetrance. However, this method suffers from a variety of drawbacks. First, linkage analysis is limited by its reliance on the choice of a genetic model suitable for each studied trait. Furthermore, the resolution attainable using linkage analysis is limited, and complementary studies are required to refine the analysis of the typical 20 Mb regions initially identified through this method. In addition, linkage analyses have proven difficult when applied to complex genetic traits, such as those due to the combined action of multiple genes and/or environmental factors. Finally, linkage analysis relies on the availability of large informative families.

Other genetic strategies for the identification of disease-causing genes are based on the association of single nucleotide polymorphisms (SNPs) with disease cases. One of these strategies is the transmission/disequilibrium test (TDT) which is based on the examination of a SNP marker with affected or unaffected individuals in a nuclear family comprised of at least two parents and a disease-case (i.e., a trio).

However, since many complex diseases are of late-onset, (e.g., Alzheimer's and Parkinson's diseases, coronary heart disorders, type II diabetes mellitus and various cancers), the parents of the affected individuals are often not alive or not available for a family-based genetic study using the linkage analysis or the TDT designs.

Population Association Studies

Complex genetic disorders may be advantageously studied using the population association studies with the case-control design. The general concept underlying this approach is the comparison of allele frequencies between cases and controls. The risk for a false-positive result is very much reduced when both cases and controls originate in the same homogenous population.

Association studies seek to analyze the distributions of chromosomes in populations of unrelated (at least not directly related) individuals. A key assumption in this type of study is that genetic alleles that result in susceptibility for a common trait arose by ancient mutational events on chromosomes that have been passed down through many generations in the population. These alleles can become common throughout the population, in part because the trait they influence, if deleterious, is only expressed in a fraction of those individuals who carry them. Identification of these “ancestral” chromosomes is made difficult by the fact that genetic markers are likely to have become separated from the trait susceptibility allele through the process of recombination, except in regions of DNA which immediately surround the disease allele. The identities of genetic markers contained within the fragments of DNA surrounding a susceptibility allele will be the same as those from the ancestral chromosome on which the allele arose. Therefore, individuals from the population who express a complex trait are expected to carry the same set of genetic markers in the vicinity of a susceptibility allele more often than those who do not express the trait; that is these markers will show an association with the trait.

One of the advantages of the case-control design over the TDT design for studying markers distinct from the disease variants, is the number of cases needed to reveal significance. The number of trios (i.e., two parents and a disease case) needed for the TDT design is virtually equal to the number of cases needed for case-control studies with an equal number of cases and controls [McGinnis, R., Shifman, S. and Darvasi, A. (2002). Power and efficiency of the TDT and case-control design for association scans. Behavior. Genetics 32: 135-144]. This implies that in case-control studies, two-thirds of the samples, compared to the TDT design, are required to achieve the same power, suggesting greater genotyping efficiency with the case-control design. Moreover, unrelated controls are often much easier to collect than both parents of a disease case. Furthermore, unlike the TDT, the case-control design can also use DNA pooling strategies [Arnheim et al., (1985). Use of pooled DNA samples to detect linkage disequilibrium of polymorphic restriction fragments and human disease: Studies of the HLA class II loci. Proc. Natl. Acad. Sci. USA 82: 6970-6974; Barcellos et al., (1997). Association mapping of disease loci by use of a pooled DNA genomic screen. Am. J. Hum. Genet. 61: 734-747] and thus may reduce the huge number of genotyping reactions required for association scans by several orders of magnitude. This again argues that a case-control study will often be much cheaper and faster to implement than a similar-powered TDT design.

The Catechol-O-methyltransferase (COMT)

Inconclusive Association Studies with Schizophrenia

Accumulating evidence suggest an association between schizophrenia susceptibility and hemizygous deletions of 22q11. One of the genes located within the 22q11 microdeletion is the catechol-O-methyltransferase (COMT) gene. This enzyme (E.C. 2.1.1.6) degrades catecholamine neurotransmitters including dopamine. Since altered dopaminergic transmission has long been believed to play a role in the development of schizophrenia [Carlsson A. (1988). The current status of the dopamine hypothesis of schizophrenia. Neuropsychopharmacology. 1: 179-861 this gene has been extensively studied as a candidate gene for schizophrenia. Many of these studies have focused on a non-synonymous Val/Met polymorphism at position 108 of SEQ ID NO:30 or at position 158 of SEQ ID NO:29, which leads to a several-fold reduction in COMT enzymatic activity [Lachman H M, Morrow B, Shprintzen R, Veit S, Parsia S S, Faedda G, Goldberg R, Kucherlapati R, Papolos D F. (1996). Association of codon 108/158 catechol-O-methyltransferase gene polymorphism with the psychiatric manifestations of velo-cardio-facial syndrome. Am J Med Genet. 67: 468-72; Lotta T, Vidgren J, Tilgmann C, Ulmanen I, Melen K, Julkunen I, Taskinen J. (1995). Kinetics of human soluble and membrane-bound catechol O-methyltransferase: a revised mechanism and description of the thermolabile variant of the enzyme. Biochemistry. 34: 4202-10; Kunugi et al. (1997). Psychiatr. Genet. 7: 97-101; Li et al. (1996). Psychiatr. Genet. 6: 131-133; Li et al. (2000). Mol. Psychiatry 5: 77-84; Karayiorgou et al. (1998). Biol. Psychiatry 43: 425-431; Palmatier et al. (1997). PNAS: 94: 587-592]. However, none of these studies has provided conclusive results.

Thus, there is no genetic marker available to date which is indicative of a subject's susceptibility to schizophrenia or bipolar disorder.

Inconclusive Association Studies with Breast Cancer

The COMT polymorphism V158M of SEQ ID NO:29 (or V108M of SEQ ID NO:30) has been extensively investigated in several breast cancer genetic association studies. Several studies have found an association of the low activity form of the COMT enzyme (M158 of SEQ ID NO:29 or M108 of SEQ ID NO:30) with breast cancer [Thompson, P. A., P. G. Shields, et al. (1998). Genetic polymorphisms in catechol-O-methyltransferase, menopausal status, and breast cancer risk. Cancer Res 58(10): 2107-10; Lavigne, J. A., K. J. Helzlsouer, et al. (1997). An association between the allele coding for a low activity variant of catechol-O-methyltransferase and the risk for breast cancer. Cancer Res 57(24): 5493-7; Matsui, A., T. Ikeda, et al. (2000). Progression of human breast cancers to the metastatic state is linked to genotypes of catechol-O-methyltransferase. Cancer Lett 150(1): 23-31; Yim, D. S., S. K. Parkb, et al. (2001). Relationship between the Val158Met polymorphism of catechol O-methyl transferase and breast cancer. Pharmacogenetics 11(4): 279-8]. However, other studies reported no significant association between the COMT gene and breast cancer [Millikan, R. C., G. S. Pittman, et al. (1998). Catechol-O-methyltransferase and breast cancer risk. Carcinogenesis 19(11): 1943-7; Bergman-Jungestrom, M. and S. Wingren (2001). Catechol-O-Methyltransferase (COMT) gene polymorphism and breast cancer risk in young women. Br J Cancer 85(6): 859-62; Hamajima, N., K. Matsuo, et al. (2001). Limited association between a catechol-O-methyltransferase (COMT) polymorphism and breast cancer risk in Japan. Int J Clin Oncol 6(1): 13-8; Kocabas, N. A., S. Sardas, et al. (2002). Cytochrome P450 CYP1B1 and catechol O-methyltransferase (COMT) genetic polymorphisms and breast cancer susceptibility in a Turkish population. Arch Toxicol. 76(11): 643-9]. Thus, there is no definitive finding associating the COMT gene with breast cancer. In addition, no studies on association between the COMT gene and colorectal cancer have been reported.

There is thus a widely recognized need for, and it would be highly advantageous to have, indicative markers for a subject's susceptibility to schizophrenia, bipolar disorder, breast cancer and colorectal cancer which will be used for the diagnosis of these diseases and will contribute to the development of medicaments for treating the diseases.

### SUMMARY OF THE INVENTION

According to one aspect of the present invention there is provided a method of determining a predisposition of a subject to develop schizophrenia. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with schizophrenia, thereby determining the predisposition of the subject of developing schizophrenia.

According to another aspect of the present invention there is provided a kit for determining a predisposition of a subject to develop schizophrenia. The kit according to this aspect of the present invention comprises a packaging material packaging at least one reagent, the at least one reagent is for determining a presence in a homozygous or heterozygous form, or an absence, of at least one genotype in the COMT locus or in neighboring loci, the neighboring loci are in linkage disequilibrium with the COMT locus, the genotype having been associated with schizophrenia. The kit further comprises a notification in or on the packaging material identifying the kit for use in determining a predisposition of developing schizophrenia. Preferably, the notification also provides for instructions of using the kit in determining a predisposition of developing schizophrenia.

According to yet another aspect of the present invention there is provided a method of assisting in diagnosing schizophrenia in a subject in need thereof. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with schizophrenia, thereby assisting in diagnosing schizophrenia in the subject.

According to still another aspect of the present invention there is provided a kit for assisting in diagnosing schizophrenia in a subject in need thereof. The kit according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with schizophrenia. The kit further comprises a notification in or on the packaging material identifying the kit for use in assisting in diagnosing schizophrenia in a subject. Preferably, the notification also provides for instructions of using the kit in assisting in diagnosing schizophrenia of a subject.

According to an additional aspect of the present invention there is provided a method of determining a predisposition to develop schizophrenia in a female subject. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with schizophrenia in females in higher association than in males, thereby determining the predisposition of the female subject of developing schizophrenia.

According to yet an additional aspect of the present invention there is provided a kit for determining a predisposition to develop schizophrenia in a female subject. The kit, according to this aspect of the present invention comprises a packaging material packaging at least one reagent, the at least one reagent for determining a presence in a homozygous or heterozygous form, or an absence, of at least one genotype in the COMT locus or in neighboring loci, the neighboring loci are in linkage disequilibrium with the COMT locus, the genotype having been associated with schizophrenia in females in higher association than in males. The kit further comprises a notification in or on the packaging material, the notification identifying the kit for use in determining a predisposition of a female subject of developing schizophrenia. Preferably, the notification also provides for instructions of using the kit in determining the predisposition of a female subject of developing schizophrenia.

According to still an additional aspect of the present invention there is provided a method of assisting in diagnosing schizophrenia in a female subject in need thereof. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with schizophrenia in females in higher association than in males, thereby assisting in diagnosing schizophrenia in the female subject.

According to a further aspect of the present invention there is provided a kit for assisting in diagnosing schizophrenia in a female subject in need thereof. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with schizophrenia in females in higher association than in males. The kit further comprises a notification in or on the packaging material, the notification identifying the kit for use in assisting in diagnosing schizophrenia in a female subject. Preferably, the notification also provides for instructions of using the kit in assisting in diagnosing schizophrenia of a female subject.

According to yet a: further aspect of the present invention there is provided a method of determining a predisposition to develop schizophrenia in a male subject. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with schizophrenia in males in higher association than in females, thereby determining the predisposition of the male subject of developing schizophrenia.

According to still a further aspect of the present invention there is provided a kit for determining a predisposition to develop schizophrenia in a male subject. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with schizophrenia in males in higher association than in females. The kit further comprises a notification in or on the packaging material, the notification identifying the kit for use in determining a predisposition of a male subject of developing schizophrenia. Preferably, the notification also provides for instructions of using the kit in determining the predisposition of a male subject of developing schizophrenia.

According to still a further aspect of the present invention there is provided method of assisting in diagnosing schizophrenia in a male subject in need thereof. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with schizophrenia in males in higher association than in females, thereby assisting in diagnosing schizophrenia in the male subject.

According to still a further aspect of the present invention there is provided kit for assisting in diagnosing schizophrenia in a male subject in need thereof. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with schizophrenia in males in higher association than in females. The kit further comprises a notification in or on the packaging material, the notification identifying the kit for use in assisting in diagnosing schizophrenia in a male subject. Preferably, the notification also provides for instructions of using the kit in assisting in diagnosing schizophrenia of a male subject.

According to still a further aspect of the present invention there is provided a method of determining a predisposition to develop bipolar disorder in a subject. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with bipolar disorder, thereby determining the predisposition of the subject of developing bipolar disorder.

According to still a further aspect of the present invention there is provided a kit for determining a predisposition to develop bipolar disorder in a subject. The kit, according to this aspect of the present invention comprises a packaging material packaging at least one reagent, the at least one reagent for determining a presence in a homozygous or heterozygous form, or an absence, of at least one genotype in the COMT locus or in neighboring loci, the neighboring loci are in linkage disequilibrium with the COMT locus, the genotype having been associated with bipolar disorder. The kit further comprises a notification in or on the packaging material, the notification identifying the kit for use in determining a predisposition of a subject of developing bipolar disorder. Preferably, the notification also provides for instructions of using the kit in determining the predisposition of a subject of developing bipolar disorder.

According to still a further aspect of the present invention there is provided a method of determining a predisposition to develop bipolar disorder in a female subject. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with bipolar disorder in females in higher association than in males, thereby determining the predisposition of the female subject of developing bipolar disorder.

According to still a further aspect of the present invention there is provided a kit for determining a predisposition to develop bipolar disorder in a female subject. The kit, according to this aspect of the present invention comprises a packaging material packaging at least one reagent, the at least one reagent for determining a presence in a homozygous or heterozygous form, or an absence, of at least one genotype in the COMT locus or in neighboring loci, the neighboring loci are in linkage disequilibrium with the COMT locus, the genotype having been associated with bipolar disorder in females in higher association than in males. The kit further comprises a notification in or on the packaging material, the notification identifying the kit for use in determining a predisposition of a female subject of developing bipolar disorder. Preferably, the notification also provides for instructions of using the kit in determining the predisposition of a female subject of developing bipolar disorder.

According to still a further aspect of the present invention there is provided a method of assisting in diagnosing bipolar disorder in a subject in need thereof. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with bipolar disorder, thereby assisting in diagnosing the bipolar disorder in the subject.

According to still a further aspect of the present invention there is provided a kit for assisting in diagnosing bipolar disorder in a subject in need thereof. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with bipolar disorder. The kit further comprises a notification in or on the packaging material, the notification identifying the kit for use in assisting in diagnosing bipolar disorder in a subject. Preferably, the notification also provides for instructions of using the kit in assisting in diagnosing bipolar disorder in a subject.

According to still a further aspect of the present invention there is provided a method of assisting in diagnosing bipolar disorder in a female subject in need thereof. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with bipolar disorder in females in higher association than in males, thereby assisting in diagnosing the bipolar disorder in the female subject.

According to still a further aspect of the present invention there is provided a kit for assisting in diagnosing bipolar disorder in a female subject in need thereof. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with bipolar disorder in females in higher association than in males. The kit further comprises a notification in or on the packaging material, the notification identifying the kit for use in assisting in diagnosing bipolar disorder in a female subject. Preferably, the notification also provides for instructions of using the kit in assisting in diagnosing bipolar disorder in a female subject.

According to still a further aspect of the present invention there is provided a method of determining a predisposition to develop breast cancer in a subject. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with breast cancer, thereby determining the predisposition of the subject of developing breast cancer.

According to still a further aspect of the present invention there is provided a kit for determining a predisposition to develop breast cancer in a subject. The kit, according to this aspect of the present invention comprises a packaging material packaging at least one reagent, the at least one reagent for determining a presence in a homozygous or heterozygous form, or an absence, of at least one genotype in the COMT locus or in neighboring loci, the neighboring loci are in linkage disequilibrium with the COMT locus, the genotype having been associated with breast cancer. The kit further comprises a notification in or on the packaging material, the notification identifying the kit for use in determining a predisposition of a subject of developing breast cancer. Preferably, the notification also provides for instructions of using the kit in determining a predisposition of a subject of developing breast cancer.

According to still a further aspect of the present invention there is provided a method of assisting in diagnosing breast cancer in a subject in need thereof. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with breast cancer, thereby assisting in diagnosing the breast cancer in the subject.

According to still a further aspect of the present invention there is provided a kit for assisting in diagnosing breast cancer in a subject in need thereof. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with breast cancer. The kit further comprises a notification in or on the packaging material, the notification identifying the kit for use in assisting in diagnosing breast cancer in a subject. Preferably, the notification also provides for instructions of using the kit in assisting in diagnosing breast cancer in a subject.

According to still a further aspect of the present invention there is provided a method of determining a prognosis of a breast cancer in a subject in need thereof. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with lymph node metastases of breast cancer, thereby determining the prognosis of the breast cancer in the subject.

According to still a further aspect of the present invention there is provided a kit for determining a prognosis of a breast cancer in a subject in need thereof. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with lymph node metastases of breast cancer. The kit further comprises a notification in or on the packaging material, the notification identifying the kit for use in determining a prognosis of a breast cancer in a subject. Preferably, the notification also provides for instructions of using the kit in determining the prognosis of the breast cancer in a subject.

According to still a further aspect of the present invention there is provided a method of determining a predisposition to develop colorectal cancer in a subject. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with colorectal cancer, thereby determining the predisposition of the subject of developing colorectal cancer.

According to still a further aspect of the present invention there is provided a kit for determining a predisposition to develop colorectal cancer in a subject. The kit, according to this aspect of the present invention comprises a packaging material packaging at least one reagent, the at least one reagent for determining a presence in a homozygous or heterozygous form, or an absence, of at least one genotype in the COMT locus or in neighboring loci, the neighboring loci are in linkage disequilibrium with the COMT locus, the genotype having been associated with colorectal cancer. The kit further comprises a notification in or on the packaging material, the notification identifying the kit for use in determining a predisposition of a subject of developing colorectal cancer. Preferably, the notification also provides for instructions of using the kit in determining the predisposition of a subject of developing colorectal cancer.

According to still a further aspect of the present invention there is provided a method of assisting in diagnosing colorectal cancer in a subject in need thereof. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with colorectal cancer, thereby assisting in diagnosing colorectal cancer in the subject.

According to still a further aspect of the present invention there is provided a kit for assisting in diagnosing colorectal cancer in a subject in need thereof. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with colorectal cancer. The kit further comprises a notification in or on the packaging material, the notification identifying the kit for use in assisting in diagnosing colorectal cancer in a subject. Preferably, the notification also provides for instructions of using the kit in assisting in diagnosing colorectal cancer in a subject.

According to still a further aspect of the present invention there is provided a method of determining a predisposition to develop colorectal cancer in a female subject. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with colorectal cancer in females in higher association than in males, thereby determining the predisposition of the female subject of developing colorectal cancer.

According to still a further aspect of the present invention there is provided a kit for determining a predisposition to develop colorectal cancer in a female subject. The kit, according to this aspect of the present invention comprises a packaging material packaging at least one reagent, the at least one reagent for determining a presence in a homozygous or heterozygous form, or an absence, of at least one genotype in the COMT locus or in neighboring loci, the neighboring loci are in linkage disequilibrium with the COMT locus, the genotype having been associated with colorectal cancer in females in higher association than in males. The kit further comprises a notification in or on the packaging material, the notification identifying the kit for use in determining a predisposition of a female subject of developing colorectal cancer. Preferably, the notification also provides for instructions of using the kit in determining the predisposition of the female subject of developing colorectal cancer.

According to still a further aspect of the present invention there is provided a method of assisting in diagnosing colorectal cancer in a female subject. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with colorectal cancer in females in higher association than in males, thereby assisting in diagnosing colorectal cancer in the female subject.

According to still a further aspect of the present invention there is provided a kit for assisting in diagnosing colorectal cancer in a female subject in need thereof. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with colorectal cancer in females in higher association than in males. The kit further comprises a notification in or on the packaging material, the notification identifying the kit for use in assisting in diagnosing colorectal cancer in a female subject. Preferably, the notification also provides for instructions of using the kit in assisting in diagnosing colorectal cancer in a female subject.

According to still a further aspect of the present invention there is provided a method of predicting drug responsiveness of a subject having schizophrenia to a schizophrenia drug. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with drug responsiveness to the drug, thereby predicting drug responsiveness of the subject to the drug.

According to still a further aspect of the present invention there is provided a kit for predicting drug responsiveness of a subject having schizophrenia to a schizophrenia drug. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with schizophrenia. The kit further comprises a notification in or on the packaging material, the notification identifying the kit for use in predicting drug responsiveness of a subject having schizophrenia to the drug. Preferably, the notification also provides for instructions of using the kit in assisting in predicting drug responsiveness of a subject having schizophrenia to the drug.

According to still a further aspect of the present invention there is provided a method of predicting drug responsiveness of a subject having a given mental disorder to a mental disorder drug. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with drug responsiveness to the drug in at least one mental disorder, thereby predicting drug responsiveness of the subject having the given mental disorder to the drug.

According to still a further aspect of the present invention there is provided a kit for predicting drug responsiveness of a subject having a given mental disorder to a mental disorder drug. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with drug responsiveness to the drug in at least one mental disorder. The kit further comprises a notification in or on the packaging material, the notification identifying the kit for use in predicting drug responsiveness of a subject having the given mental disorder to the drug. Preferably, the notification also provides for instructions of using the kit in assisting in predicting drug responsiveness of a subject having the given mental disorder to the drug.

According to still a further aspect of the present invention there is provided a method of identifying a genetic association with, or a genetic cause to, varying drug responsiveness to a schizophrenia drug. The method according to this aspect of the present invention comprises determining via a population association study an association between a presence in a homozygous or heterozygous form, or an absence, of at least one genotype in the COMT locus or in neighboring loci, the neighboring loci are in linkage disequilibrium with the COMT locus, and responsiveness or non-responsiveness to the schizophrenia drug, thereby identifying a genetic association with, or a genetic cause to, the varying drug responsiveness to the schizophrenia drug.

According to still a further aspect of the present invention there is provided a kit for identifying a genetic association with, or a genetic cause to, varying drug responsiveness to a schizophrenia drug. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with schizophrenia. The kit further comprises a notification in or on the packaging material, the notification identifying the kit for use in identifying a genetic association with, or a genetic cause to, varying drug responsiveness to the drug. Preferably, the notification also provides for instructions of using the kit in identifying a genetic association with, or a genetic cause to, varying drug responsiveness to the drug.

According to still a further aspect of the present invention there is provided a method of identifying a genetic association with, or a genetic cause to, varying drug responsiveness to a mental disorder drug. The method according to this aspect of the present invention comprises determining via a population association study an association between a presence in a homozygous or heterozygous form, or an absence, of at least one genotype in the COMT locus or in neighboring loci, the neighboring loci in linkage disequilibrium with the COMT locus, and responsiveness or non-responsiveness to the mental disorder drug, thereby identifying a genetic association with, or a genetic cause to, the varying drug responsiveness to the mental disorder drug.

According to still a further aspect of the present invention there is provided a kit for identifying a genetic association with, or a genetic cause to, varying drug responsiveness of a subject having a given mental disorder to a mental disorder drug. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with drug responsiveness to the drug in at least one mental disorder. The kit further comprises a notification in or on the packaging material, the notification identifying the kit for use in identifying a genetic association with, or a genetic cause to, varying drug responsiveness of a subject having the given mental disorder to the drug. Preferably, the notification also provides for instructions of using the kit in identifying a genetic association with, or a genetic cause to, varying drug responsiveness of a subject having the given mental disorder to the drug.

According to further features in preferred embodiments of the invention described below, the schizophrenia drug is selected from the group consisting of thioridazine, clozapine and haloperidol.

According to still further features in the described preferred embodiments the mental disorder drug is selected from the group consisting thioridazine, clozapine, haloperidol, fluphenazine, cholrpromazine, risperidone, levomepromazine, perhenazine, chlorprothixene, pimozide, sulpiride, olanzapine, zuclopenthixol, amitriptyline, imipramine, clomipramine, desipramine, doxepin, mianserin, maprotiline, phenelzine, fluoxetine, trazodone, fluvoxamine, sertraline, paroxetine, reboxetine, citalopram, nefazodone, venlafoxine, lithium salts, carbamazapine, valproic acid, and clonazepam.

According to still further features in the described preferred embodiments the mental disorder is selected from the group consisting of schizophrenia, schizoaffective disorder, bipolar disorder, depression, obsessive compulsive disorder, panic disorder, agoraphobia, specific phobia, social phobia, post-traumatic stress disorder, pain disorder, anxiety, somatization disorder, anorexia nervosa, bulimia, and nervosa.

According to still further features in the described preferred embodiments the at least one genotype in the COMT locus is a guanine nucleotide—and/or an adenosine nucleotide—containing allele of SNP rs4680 set forth in SEQ ID NO:5;

According to still further features in the described preferred embodiments the at least one genotype in the COMT locus is a guanine nucleotide—and/or an adenosine nucleotide—containing allele of SNP rs165599 set forth in SEQ ID NO:9.

According to still further features in the described preferred embodiments the at least one genotype in the COMT locus is a cytosine nucleotide—and/or a thymine nucleotide—containing allele of SNP rs737865 set forth in SEQ ID NO: 1.

According to still further features in the described preferred embodiments the at least one genotype in the COMT locus is an allelic haplotype comprising at least two of: a cytosine nucleotide—and/or a thymine nucleotide—containing allele of SNP rs737865 set forth in SEQ ID NO:1; a guanine nucleotide—and/or an adenosine nucleotide containing allele of SNP rs4680 set forth in SEQ ID NO:5; and a guanine nucleotide—and/or an adenosine nucleotide containing allele of SNP rs165599 set forth in SEQ ID NO:9.

According to still further features in the described preferred embodiments the at least one reagent is selected from the group consisting of at least one oligonucleotide, at least one antibody and a DNA chip.

According to still further features in the described preferred embodiments the at least one oligonucleotide has a sequence selected differentially hybridizeable to at least one allele of an SNP selected from the group consisting of rs4680, rs165599 and rs737865, the at least one oligonucleotide can differentiate between polymorphs of the SNP via differential hybridization.

According to still further features in the described preferred embodiments the at least one oligonucleotide has a sequence selected hybridizeable adjacent to an SNP selected from the group consisting of rs4680, rs165599 and rs737865, the at least one oligonucleotide can differentiate between polymorphs of the SNP via a differential template dependent primer extension reaction or a differential template dependent ligation reaction.

According to still further features in the described preferred embodiments the at least one oligonucleotide has a sequence selected hybridizeable adjacent to an SNP selected from the group consisting of rs4680, rs165599 and rs737865, the at least one oligonucleotide can be used to amplify polymorphs of the SNP via an amplification reaction.

According to still further features in the described preferred embodiments the at least one antibody is differentially interactable with at least one form of a COMT protein encoded by a COMT allele having an SNP rs4680, the at least one antibody can differentiate between polymorphs of the COMT protein via differential antibody interaction.

According to still further features in the described preferred embodiments the DNA chip comprises attached thereto at least one oligonucleotide having a sequence selected differentially hybridizeable to at least one allele of an SNP selected from the group consisting of rs4680, rs165599 and rs737865, the at least one oligonucleotide can differentiate between polymorphs of the SNP via differential hybridization.

According to still further features in the described preferred embodiments at least one reagent is designed for performing a detection method selected from the group consisting of a signal amplification method, a direct detection method and detection of at least one sequence change.

According to still further features in the described preferred embodiments the signal amplification method amplifies a molecule selected from the group consisting of a DNA molecule and an RNA molecule.

According to still further features in the described preferred embodiments the signal amplification method is selected from the group consisting of PCR, LCR (LAR), Self-Sustained Synthetic Reaction (3SR/NASBA) and Q-Beta (Qβ) Replicase reaction.

According to still further features in the described preferred embodiments the direct detection method is selected from the group consisting of a cycling probe reaction (CPR) and a branched DNA analysis.

According to still further features in the described preferred embodiments the at least one sequence change employs a method selected from the group consisting of restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis and Dideoxy fingerprinting (ddF).

According to still further features in the described preferred embodiments the at least one reagent is designed for performing an immunological detection method for a COMT protein encoded by the COMT locus.

According to still further features in the described preferred embodiments the immunological detection method is selected from the group consisting of a radio-immunoassay (RIA), an enzyme linked immunosorbent assay (ELISA), a western blot, an immunohistochemical analysis, and fluorescence activated cell sorting (FACS).

According to still a further aspect of the present invention there is provided a method of identifying novel drugs for treatment of schizophrenia. The method according to this aspect of the present invention comprises incubating a catechol-O-methyltransferase protein or cells expressing catechol-O-methyltransferase with potential drugs and selecting for at least one drug of the potential drugs that modulates catechol-O-methyltransferase activity or expression, thereby identifying novel drugs for treatment of schizophrenia.

According to still a further aspect of the present invention there is provided a method of treating schizophrenia. The method according to this aspect of the present invention comprises administering to a subject in need thereof a therapeutically effective amount of a drug for schizophrenia, the drug for schizophrenia having been identified using the method described hereinabove.

According to still a further aspect of the present invention there is provided a method of identifying novel drugs for treatment of bipolar disorder. The method according to this aspect of the present invention comprises incubating a catechol-O-methyltransferase protein or cells expressing catechol-O-methyltransferase with potential drugs and selecting for at least one drug of the potential drugs that modulates catechol-O-methyltransferase activity or expression, thereby identifying novel drugs for treatment of bipolar disorder.

According to still a further aspect of the present invention there is provided a method of treating bipolar disorder. The method according to this aspect of the present invention comprises administering to a subject in need thereof a therapeutically effective amount of a drug for bipolar disorder, the drug for bipolar disorder having been identified using the method described hereinabove.

According to still a further aspect of the present invention there is provided a method of identifying novel drugs for treatment of breast cancer. The method according to this aspect of the present invention comprises incubating a catechol-O-methyltransferase protein or cells expressing catechol-O-methyltransferase with potential drugs and selecting for at least one drug of the potential drugs that modulates catechol-O-methyltransferase activity or expression, thereby identifying novel drugs for treatment of breast cancer.

According to still a further aspect of the present invention there is provided a method of treating breast cancer. The method according to this aspect of the present invention comprises administering to a subject in need thereof a therapeutically effective amount of a drug for breast cancer, the drug for breast cancer having been identified using the method described hereinabove.

According to still a further aspect of the present invention there is provided a method of identifying novel drugs for treatment of colorectal cancer. The method according to this aspect of the present invention comprises incubating a catechol-O-methyltransferase protein or cells expressing catechol-O-methyltransferase with potential drugs and selecting for at least one drug of the potential drugs that modulates catechol-O-methyltransferase activity or expression, thereby identifying novel drugs for treatment of colorectal cancer.

According to still a further aspect of the present invention there is provided a method of treating colorectal cancer. The method according to this aspect of the present invention comprises administering to a subject in need thereof a therapeutically effective amount of a drug for colorectal cancer, the drug for colorectal cancer having been identified using the method described hereinabove.

According to still further features in the described preferred embodiments the at least one drug of the potential drugs is selected from the group consisting of a peptide, a polynucleotide and a small molecule.

According to still further features in the described preferred embodiments the polynucleotide is selected from the group consisting of an antisense oligonucleotide, an siRNA, a ribozymes and a DNAzyme.

According to still further features in the described preferred embodiments the peptide is selected from the group consisting of a small peptide, a polypeptide and an antibody.

According to still a further aspect of the present invention there is provided a method of treating and/or preventing schizophrenia. The method according to this aspect of the present invention comprises administering to a subject in need thereof a therapeutically effective amount of at least one agent capable of inhibiting COMT protein expression or activity.

According to still a further aspect of the present invention there is provided a method of preventing breast cancer. The method according to this aspect of the present invention comprises administering to a subject in need thereof a therapeutically prophylactically effective amount of at least one agents capable of inhibiting COMT protein expression or activity.

According to still a further aspect of the present invention there is provided a method of treating and/or preventing colorectal cancer. The method according to this aspect of the present invention comprises administering to a subject in need thereof a therapeutically effective amount of at least one agents capable of inhibiting COMT protein expression or activity.

According to still further features in the described preferred embodiments the at least one agent is selected from the group consisting of an anti COMT antibody, a polynucleotide encoding an intracellular anti COMT antibody, an anti COMT antisense molecule, an anti COMT siRNA, an anti COMT ribozyme, an anti COMT DNAzyme, and a COMT inhibitor.

According to still further features in the described preferred embodiments the COMT inhibitor is selected from the group consisting of 2′-fluoro-3,4-dihydroxy-5-nitrobenzophenone, 3,4-dihydroxy-5-nitrophenyl derivatives, catechol derivatives, 3,4-dihydroxy4′-methyl-5-nitrobenzophenone.

The present invention successfully addresses the shortcomings of the presently known configurations by providing methods and kits useful for predisposition, diagnosis and treatment of schizophrenia, bipolar disorder, breast cancer and colorectal cancer.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

The following terms and phrases shall have the following meaning:

As used herein, the term “predisposition” refers to a situation of susceptibility to develop a disorder or disease. An individual with a predisposition to a disorder or disease is more likely than an individual without the predisposition to the disorder or disease to develop the disorder or disease.

Methods of “determining a predisposition” of an individual to a disorder or disease are used in genetic counseling, especially prior to making a decision to conceive a child. Also, application of methods for detecting genotypes of SNPs in the COMT locus as described herein can be applied to the parents of an individual with any of the disorders described hereinabove. The transmission of the polymorphism refers to the distribution of a COMT polymorphic allele from a parent to an offspring. When a parent is heterozygous for a polymorphism, for example, the guanine/adenosine (G/A) polymorphism of SNP rs4680 set forth in SEQ ID NO:5, the likelihood that the guanine allele will be transmitted is usually the same as the likelihood that the adenosine allele will be transmitted to the offspring. A genetic locus that is linked to another locus shows equal allelic transmission.

The term “polymorphism” refers to the occurrence of two or more genetically determined variant forms (alleles) of a particular nucleic acid at a frequency where the rarer (or rarest) form could not be maintained by recurrent mutation alone. A single nucleotide polymorphism (SNP) results from a single base difference between related alleles at the same genetic locus. Exemplary nucleotide polymorphisms include the G/A polymorphism of SNP rs4680 set forth in SEQ ID NO:5.

The term “homozygous” refers to an individual having two identical alleles of a certain polymorphism. A non-limiting example is an individual having the GG genotype of SNP rs4680. This individual is referred to as an homozygote for this SNP.

The term “heterozygous” refers to an individual having two different alleles of a certain polymorphism. For example, an individual having the A/G genotype of SNP rs4680 is referred as an heterozygote individual for this SNP.

The phrase “absence of genotype” is used herein to describe the negative result of a specific genotype determination test. For example, if the genotype determination test is suitable for the identification of guanine nucleotide—containing allele of SNP rs4680 set forth in SEQ ID NO:5, and the individual on which the test is performed is homozygote for the adenosine nucleotide—containing allele of SNP rs4680, then the result of the test will be “absence of genotype”.

SNPs may be detectable at the protein level if the nucleotide change is in the coding sequence and encodes a different amino acid residue, creating two “polymorphs” of the protein. A non-limiting example of an amino acid change is the Val/Met polymorphism at position 158 and/or 108 of the COMT protein set forth in SEQ ID NO:29 and/or SEQ ID NO:30, respectively, which results from the G/A polymorphism of SNP rs4680 set forth in SEQ ID NO:5 in the COMT gene.

As used herein the phrase “COMT locus” refers to the location of a DNA stretch in the human genome corresponding to a gene coding for the COMT enzyme.

The phrase “neighboring loci” is used herein to describe DNA sequences (either genes or intergene sequences) that are in close vicinity of the COMT locus and that include other SNPs that are in linkage disequilibrium with SNPs in the COMT locus.

The phrase “linkage disequilibrium” (LD) is used to describe the statistical correlation between two neighboring polymorphic genotypes. Typically, LD refers to the correlation between the alleles of a random gamete at the two loci, assuming Hardy-Weinberg equilibrium (statistical independence) between gametes. LD is quantified with either Lewontin's parameter of association (D′) or with Pearson correlation coefficient (r) [Devlin B, Risch N. (1995). A comparison of linkage disequilibrium measures for fine-scale mapping. Genomics. 29: 311-322.]. Two loci with a LD value of 1 are said to be in complete LD. At the other extreme, two loci with a LD value of 0 are termed to be in linkage equilibrium. Linkage disequilibrium is calculated following the application of the expectation maximization algorithm (EM) for the estimation of haplotype frequencies [Slatkin M, Excoffier L. (1996). Testing for linkage disequilibrium in genotypic data using the Expectation-Maximization algorithm. Heredity. 76: 377-83.]. LD values according to the present invention for neighboring genotypes/loci are selected above 0.1, preferably, above 0.2, more preferable above 0.5, more preferably, above 0.6, still more preferably, above 0.7, preferably, above 0.8, more preferably above 0.9, ideally about 1.0 to 1.0.

The phrase “genetic association” refers to a linkage between certain genetic markers such as SNPs, di-nucleotide or tri-nucleotide repeats, and the like with a certain phenotype such as disease, trait, drug responsiveness and the like.

The phrase “genetic cause” refers to the genetic basis of a phenomenon such as a disease or disorder or varying responsiveness to a drug. As the genetic make-up of an individual determines the individual various traits, including individual's risk of developing a disease, it also determines the response exerted in an individual to a specific drug.

The phrase “drug responsiveness” is used herein in reference to genotypes in the COMT locus which determine the specific response an individual has to a specific drug. The response to the drug can be high- or low-efficiency, no-effect or adverse-effect. The knowledge of the specific genotype which predicts drug responsiveness can be applied to dosing and/or drug selection, can avoid adverse reactions or therapeutic failure and thus enhance therapeutic or prophylactic efficiency when treating a subject.

The phrase “mental disorder” refers to any disorder affecting an individual behavior which has a central nervous system etiology.

The term “treating” refers to inhibiting or arresting the development of a disease, disorder or condition and/or causing the reduction, remission, or regression of a disease, disorder or condition. Those of skill in the art will understand that various methodologies and assays can be used to assess the development of a disease, disorder or condition, and similarly, various methodologies and assays may be used to assess the reduction, remission or regression of a disease, disorder or condition.

As used herein, the term “preventing” refers to keeping a disease, disorder or condition from occurring in a subject who may be at risk for the disease, but has not yet been diagnosed as having the disease.

As used herein, a “subject in need” refers to an individual who has been diagnosed with or is suspected of having a disease, disorder or condition, or who is predisposed to a disease, disorder or condition. Those of skill in the art will understand that a variety of methods may be used to determine a subject at risk for a disease, and that whether a subject is at risk for a disease will depend on a variety of factors known to those of skill in the art, including genetic make-up of the subject, age, body weight, sex, diet, general health, occupation, exposure to environmental conditions, marital status, familial history and the like.

As used herein, “administering to a subject” refers to means for providing a compound that modulates COMT activity and/or expression to a patient, using any suitable route, e.g., oral, sublingual intravenous, subcutaneous, transcutaneous, intramuscular, intracutaneous, intrathecal, epidural, intraoccular, intracranial, inhalation, rectal, vaginal, and the like administration. Administration of compounds in the form of creams, lotions, tablets, capsules, pellets, dispersible powders, granules, suppositories, syrups, elixirs, lozenges, injectable solutions, sterile aqueous or non-aqueous solutions, suspensions or emulsions, patches, and the like, is also contemplated. The active ingredients may be compounded with non-toxic, pharmaceutically acceptable carriers including, glucose, lactose, gum acacia, gelatin, mannitol, starch paste, magnesium trisilicate, talc, corn starch, keratin, colloidal silica, potato starch, urea, dextrans, and the like.

As used herein, the phrase “therapeutically effective amount”, when used in reference to the invention methods employing compounds that modulate COMT activity and/or expression, refers to a dose of compound sufficient to provide circulating concentrations high enough to impart a beneficial effect on the recipient thereof. The specific therapeutically effective dose level for any particular patient will depend upon a variety of factors including the disorder being treated, the severity of the disorder, the activity of the specific compound used, the route of administration, the rate of clearance of the specific compound, the duration of treatment, the drugs used in combination or coincident with the specific compound, the age, body weight, sex, diet and general health of the patient, and like factors well known in the medical arts and sciences. Dosage levels typically fall in the range of about 0.001 up to 100 mg/kg/day; with levels in the range of about 0.05 up to 10 mg/kg/day being preferred.

The phrase “therapeutically prophylactically effective amount” refers to the amount of anti COMT agent used in order to prevent the appearance of a disease in a subject predisposed to the disease, yet is not yet inflicted with the disease.

The term “kit” refers to the combination of packaging material, reagents and a notification of use. The kit may be approved by a regulatory agency, such as the FDA, for the use identified in the notification. The notification may also include instructions to use.

The term “packaging material” refers to paper, plastic, foil, foamed substances and other materials commonly used in the packaging of chemical reagents into kits, in the form of tubes, containers, holders, wrappers, etc.

The term “notification” refers to the print, in any language, identifying the use of a kit and/or instructions of how to use the kit for that use.

### DESCRIPTION OF THE PREFERRED EMBODIMENTS

The present invention is of (i) the use of SNPs in the COMT locus and neighboring loci for the identification of genes related to, and for determining a risk of developing schizophrenia, bipolar disorder, breast cancer and colorectal cancer; (ii) methods of identifying novel drugs for the treatment and/or prevention of schizophrenia, bipolar disorder, breast cancer and colorectal cancer; (iii) methods of treating and/or preventing schizophrenia, bipolar disorder, breast cancer and colorectal cancer; and (iv) pharmacogenomic methods of determining drug responsiveness in mental disorders, schizophrenia in particular.

The principles and operation of the methods, kits and drugs according to the present invention may be better understood with reference to the accompanying descriptions and examples.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Common diseases such as mental disorders, various cancers and heart diseases often aggregate in families suggesting that there is a genetic component to the disease.

However, complex traits as well as drug efficacy, response and tolerance to toxicity are often due to the combined action of multiple genes, as well as environmental factors. The genetics of complex diseases is therefore more complicated than that of Mendelian diseases since predisposing genes do not cause the disease in every person who carries them.

While reducing the present invention to practice it was uncovered that certain genotypes (single nucleotide polymorphysms, SNPs) in the COMT locus and SNPs in neighboring loci, such as those coding for the ARVCF protein set forth in SEQ ID NO:31 (for example, SNPs selected from the NCBI data base (http://www.ncbi.nlm.nih.gov:80/SNP/snp_ref.cgi?search) such as SNPs rs3788322, rs3081759, rs2531717) and the TXNRD2 protein forms set forth in SEQ ID NOs:32, 33 and 34 (for example, SNPs selected from the NCBI data base, such as, for example, rs1139795, rs1139793 and rs3842462), which are in linkage disequilibrium with SNPs rs4680, rs165599 and/or rs737865 of the COMT locus, are highly and significantly associated with schizophrenia, bipolar disorder, breast cancer and colorectal cancer, as is further detailed and exemplified below.

Table I below summarizes the results of this study:

TABLE 1
Association and increased predisposition probabilities to schizophrenia, bipolar
disorders, breast cancer and colorectal cancer with COMT genotypes

| Allele/Genotype/double or triple | | Studied group associated |
|---|---|---|
| genotype | Disease | (increased predisposition probability) |
| rs4680 G allele | Schizophrenia | All; Male |
| rs4680 GG genotype | Schizophrenia | All (40%); Male (44%) |
| rs165599 G allele | Schizophrenia | All; Female |
| rs165599 GG genotype | Schizophrenia | All (56%); Female (123%) |
| rs737865 C allele | Schizophrenia | All; Female, Male |
| rs737865 CC genotype | Schizophrenia | All (53%); Female (83%), Male (46%) |
| rs737865/rs4680/rs165599 | Schizophrenia | All (105%); Female (82%); Male (212%) |
| CC-GG-GG | | |
| rs165599 G allele | Bipolar disorders | Females |
| rs165599 GG genotype | Bipolar disorders | Females (104%) |
| rs737865 C allele | Bipolar disorders | Females |
| rs737865 CC genotype | Bipolar disorders | Females |
| rs737865/rs165599 (CC-GG) | Bipolar disorders | Females (65%) |
| rs4680 G allele | Breast cancer | All; metastases in lymph nodes, AOO > 50, non- |
| | | familial |
| rs4680 GG genotype | Breast cancer | All (35%); metastases in lymph nodes (50%); |
| | | AOO > 50 (28%), non-familial (32%) |
| rs165599 G allele | Breast cancer | All; metastases in lymph nodes; AOO > 50, non- |
| | | familial |
| rs165599 GG genotype | Breast cancer | All (85%); metastases in lymph nodes (17%); |
| | | AOO > 50 (51%); non-familial (44%) |
| rs737865/rs4680 (TT-GG) | Breast cancer | All (155%) |
| rs4680 G allele | Colorectal | All; Females |
| | cancer | |
| rs4680 GG genotype | Colorectal | All (37%) |
| | cancer | |
| rs165599 G allele | Colorectal | All; Females |
| | cancer | |
| rs165599 GG genotype | Colorectal | All (39%); Females (80%) |
| | cancer | |
| rs737865 C allele | Colorectal | All |
| | cancer | |
| rs737865 CC genotype | Colorectal | All (39%) |
| | cancer | |
| rs4680/rs165599 (GG-GG) | Colorectal | All (56%); Females (110%) |
| | cancer | |

Studied group of schizophrenia, bipolar disorders, breast cancer and colorectal cancer associated with genotypes of the COMT locus. The increased predisposition probability is the genotype relative risk (GRR) of the homozygous risk genotype over the homozygous protective genotype.
All = males and females,
metastases in lymph nodes = an aggressive form of breast cancer,
AOO = age of onset,
non-familial = sporadic form of a disease.

Further while reducing the present invention to practice, it was uncovered that that genotypes of SNPs in the COMT locus are associated with drug responsiveness towards schizophrenia drugs.

Based on these findings, the present invention discloses methods of preventing and of treating schizophrenia, bipolar disorder, breast cancer and colorectal cancer.

### Predisposition to Schizophrenia

Previous attempts to establish an association between the COMT gene and schizophrenia resulted in inconclusive findings [Lachman H M et al., 1996, Am J Med Genet. 67: 468-72; Lotta T et al., 1995, Biochemistry. 34: 4202-10; Kunugi et al., 1997, Psychiatr. Genet. 7: 97-101; Li et al., 1996, Psychiatr. Genet. 6: 131-133; Li et al., 2000, Mol. Psychiatry 5: 77-84; Karayiorgou et al., 1998, Biol. Psychiatry 43: 425-431; Palnatier et al., 1997, PNAS: 94: 587-592]. In sharp contrast to these statistically low-power studies the present invention has established a strong and statistically significant association between genotypes in the COMT locus and increased risk to develop schizophrenia.

More specifically, the inventors of the present invention have uncovered that the G allele of SNP rs4680 set forth in SEQ ID NO:5, the G allele of SNP rs165599 set forth in SEQ ID NO:9 and the C allele of SNP rs737865 set forth in SEQ ID NO:1 are highly and significantly associated with schizophrenia. As is further described in Example 1 of the Examples section that follows and in Table 1 hereinabove, individuals homozygote to the risk COMT genotypes of SNPs rs4680, rs165599 and rs737865 have increased predisposition probabilities of 40%, 56% and 53%, respectively, over individuals having the protective genotypes of these COMT SNPs. Moreover, the predisposition probability for schizophrenia in individuals having the CC-GG-GG triple genotype of SNPs rs737865-rs4680-rs165599 is 105% higher than in individuals having the protective triple genotype. Therefore, these results suggest the use of SNPs in the COMT locus and any SNP in linkage disequilibrium with SNPs rs4680, rs165599 and rs737865 for the determination of a predisposition to schizophrenia in a subject.

Thus, according to one aspect of the present invention there is provided a method of determining a predisposition of a subject to develop schizophrenia. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with schizophrenia, thereby determining the predisposition of the subject of developing schizophrenia.

According to another aspect of the present invention there is provided a kit for determining a predisposition of a subject to develop schizophrenia. The kit according to this aspect of the present invention comprises a packaging material packaging at least one reagent, the at least one reagent is for determining a presence in a homozygous or heterozygous form, or an absence, of at least one genotype in the COMT locus or in neighboring loci, the neighboring loci are in linkage disequilibrium with the COMT locus, the genotype having been associated with schizophrenia. The kit further comprises a notification in or on the packaging material identifying the kit for use in determining a predisposition of developing schizophrenia. Preferably, the notification also provides for instructions of using the kit in determining a predisposition of developing schizophrenia.

Genotypes in the COMT locus can also assist in the diagnosis of schizophrenia. Thus, according to yet another aspect of the present invention there is provided a method of assisting in diagnosing schizophrenia in a subject in need thereof (e.g., a subject suspected of having or having a specified disease). The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with schizophrenia, thereby assisting in diagnosing schizophrenia in the subject.

According to still another aspect of the present invention there is provided a kit for assisting in diagnosing schizophrenia in a subject in need thereof. The kit according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with schizophrenia. The kit further comprises a notification in or on the packaging material identifying the kit for use in assisting in diagnosing schizophrenia in a subject. Preferably, the notification also provides for instructions of using the kit in assisting in diagnosing schizophrenia of a subject.

As is further shown in Example 1 of the Examples section which follows and in Table 1 hereinabove, the G allele of SNP rs165599 and the C allele of SNP rs737864 are highly and significantly associated with schizophrenia in females. In addition, the predisposition probability for schizophrenia in females having the GG genotype of SNP rs165599 is 123% higher than the predisposition probability in females having the AA genotype; and the predisposition probability in females having the CC genotype of SNP rs737865 is 83% higher than in females having the TT genotype. Moreover, the predisposition probability in females having the CC-GG-GG triple genotype of SNPs rs73786-rs4680-rs165599 is 82% higher than in females having the protective triple genotype.

Therefore, according to an additional aspect of the present invention there is provided a method of determining a predisposition to develop schizophrenia in a female subject. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with schizophrenia in females in higher association than in males, thereby determining the predisposition of the female subject of developing schizophrenia.

According to yet an additional aspect of the present invention there is provided a kit for determining a predisposition to develop schizophrenia in a female subject. The kit, according to this aspect of the present invention comprises a packaging material packaging at least one reagent, the at least one reagent for determining a presence in a homozygous or heterozygous form, or an absence, of at least one genotype in the COMT locus or in neighboring loci, the neighboring loci are in linkage disequilibrium with the COMT locus, the genotype having been associated with schizophrenia in females in higher association than in males. The kit further comprises a notification in or on the packaging material, the notification identifying the kit for use in determining a predisposition of a female subject of developing schizophrenia. Preferably, the notification also provides for instructions of using the kit in determining the predisposition of a female subject of developing schizophrenia.

Genotypes in the COMT locus can also assist in the diagnosis of schizophrenia in a female subject. Thus, according to still an additional aspect of the present invention there is provided a method of assisting in diagnosing schizophrenia in a female subject in need thereof. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with schizophrenia in females in higher association than in males, thereby assisting in diagnosing schizophrenia in the female subject.

According to a further aspect of the present invention there is provided a kit for assisting in diagnosing schizophrenia in a female subject in need thereof. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with schizophrenia in females in higher association than in males. The kit further comprises a notification in or on the packaging material, the notification identifying the kit for use in assisting in diagnosing schizophrenia in a female subject. Preferably, the notification also provides for instructions of using the kit in assisting in diagnosing schizophrenia of a female subject.

As is further shown in Example 1 of the Examples section which follows and in Table 1 hereinabove, the G allele and the GG genotype of SNP rs4680 and the C allele and the CC genotype of SNP rs737865 are highly and significantly associated with schizophrenia in males. Moreover, the predisposition probability for schizophrenia is 44% higher in males having the GG genotype of SNP rs4680 over males having the AA genotype, and the predisposition probability for schizophrenia is 46% higher in males having the CC genotype of SNP rs737865 over males having the TT genotype. Furthermore, the predisposition probability in males having the CC-GG-GG triple genotype of SNPs rs737865-rs4680-rs165599 is 212% higher than in males having the protective triple genotype.

Thus, according to yet a further aspect of the present invention there is provided a method of determining a predisposition to develop schizophrenia in a male subject. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with schizophrenia in males in higher association than in females, thereby determining the predisposition of the male subject of developing schizophrenia.

According to still a further aspect of the present invention there is provided a kit for determining a predisposition to develop schizophrenia in a male subject. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with schizophrenia in males in higher association than in females. The kit further comprises a notification in or on the packaging material, the notification identifying the kit for use in determining a predisposition of a male subject of developing schizophrenia. Preferably, the notification also provides for instructions of using the kit in determining the predisposition of a male subject of developing schizophrenia.

Genotypes in the COMT locus can also assist in the diagnosis of schizophrenia in a male subject. Thus, according to still a further aspect of the present invention there is provided method of assisting in diagnosing schizophrenia in a male subject in need thereof. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with schizophrenia in males in higher association than in females, thereby assisting in diagnosing schizophrenia in the male subject.

Thus, according to still a further aspect of the present invention there is provided kit for assisting in diagnosing schizophrenia in a male subject in need thereof. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with schizophrenia in males in higher association than in females. The kit further comprises a notification in or on the packaging material, the notification identifying the kit for use in assisting in diagnosing schizophrenia in a male subject. Preferably, the notification also provides for instructions of using the kit in assisting in diagnosing schizophrenia of a male subject.

### Drug Responsiveness

The inventors of the present invention have uncovered that genotypes of SNPs in the COMT locus are highly and significantly associated with drug responsiveness to schizophrenia drugs, which are commonly used in treating schizophrenia and other mental disorders. More specifically, as is further shown in Example 2 of the Examples section which follows, the GG genotype of SNP rs4680 was found to be highly and significantly associated with high efficiency of thioridazine treatment in schizophrenic females. On the other hand, the GG genotype of SNP rs4680 was significantly associated with high efficiency of clozapine in schizophrenia males. In addition, the AA genotype of SNP rs165599 was significantly associated with high efficiency of thioridazine in schizophrenic males and the AG genotype of SNP rs165599 was significantly associated with adverse effect of haloperidol treatment in schizophrenic females.

These results clearly demonstrate the use of SNPs in the COMT locus or SNPs in neighboring loci which are in linkage disequilibrium with SNPs rs4680 and rs165599 for the prediction of drug responsiveness to schizophrenia drugs. Moreover, since common schizophrenia treatment regimens include antipsychotics and neuroleptics drugs which are also widely used for treating patients having other mental disorders, in effect, the inventors of the present invention have uncovered methods for predicting the pharmacogenomic responsiveness of subjects having a variety of mental disorders to drugs used for treating these disorders using SNPs in the COMT locus and neighboring loci.

Thus, according to still a further aspect of the present invention there is provided a method of predicting drug responsiveness of a subject having schizophrenia to a schizophrenia drug. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with drug responsiveness to the drug, thereby predicting drug responsiveness of the subject to the drug.

According to still a further aspect of the present invention there is provided a kit for predicting drug responsiveness of a subject having schizophrenia to a schizophrenia drug. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with schizophrenia. The kit further comprises a notification in or on the packaging material, the notification identifying the kit for use in predicting drug responsiveness of a subject having schizophrenia to the drug. Preferably, the notification also provides for instructions of using the kit in assisting in predicting drug responsiveness of a subject having schizophrenia to the drug.

According to still a further aspect of the present invention there is provided a method of predicting drug responsiveness of a subject having a given mental disorder to a mental disorder drug. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with drug responsiveness to the drug in at least one mental disorder, thereby predicting drug responsiveness of the subject having the given mental disorder to the drug.

According to still a further aspect of the present invention there is provided a kit for predicting drug responsiveness of a subject having a given mental disorder to a mental disorder drug. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with drug responsiveness to the drug in at least one mental disorder. The kit further comprises a notification in or on the packaging material, the notification identifying the kit for use in predicting drug responsiveness of a subject having the given mental disorder to the drug. Preferably, the notification also provides for instructions of using the kit in assisting in predicting drug responsiveness of a subject having the given mental disorder to the drug.

According to still a further aspect of the present invention there is provided a method of identifying a genetic association with, or a genetic cause to, varying drug responsiveness to a schizophrenia drug. The method according to this aspect of the present invention comprises determining via a population association study an association between a presence in a homozygous or heterozygous form, or an absence, of at least one genotype in the COMT locus or in neighboring loci, the neighboring loci are in linkage disequilibrium with the COMT locus, and responsiveness or non-responsiveness to the schizophrenia drug, thereby identifying a genetic association with, or a genetic cause to, the varying drug responsiveness to the schizophrenia drug.

According to still a further aspect of the present invention there is provided a kit for identifying a genetic association with, or a genetic cause to, varying drug responsiveness to a schizophrenia drug. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with schizophrenia. The kit further comprises a notification in or on the packaging material, the notification identifying the kit for use in identifying a genetic association with, or a genetic cause to, varying drug responsiveness to the drug. Preferably, the notification also provides for instructions of using the kit in identifying a genetic association with, or a genetic cause to, varying drug responsiveness to the drug.

According to still a further aspect of the present invention there is provided a method of identifying a genetic association with, or a genetic cause to, varying drug responsiveness to a mental disorder drug. The method according to this aspect of the present invention comprises determining via a population association study an association between a presence in a homozygous or heterozygous form, or an absence, of at least one genotype in the COMT locus or in neighboring loci, the neighboring loci in linkage disequilibrium with the COMT locus, and responsiveness or non-responsiveness to the mental disorder drug, thereby identifying a genetic association with, or a genetic cause to, the varying drug responsiveness to the mental disorder drug.

According to still a further aspect of the present invention there is provided a kit for identifying a genetic association with, or a genetic cause to, varying drug responsiveness of a subject having a given mental disorder to a mental disorder drug. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with drug responsiveness to the drug in at least one mental disorder. The kit further comprises a notification in or on the packaging material, the notification identifying the kit for use in identifying a genetic association with, or a genetic cause to, varying drug responsiveness of a subject having the given mental disorder to the drug. Preferably, the notification also provides for instructions of using the kit in identifying a genetic association with, or a genetic cause to, varying drug responsiveness of a subject having the given mental disorder to the drug.

According to further features in preferred embodiments the schizophrenia drug used by the present invention can be thioridazine, clozapine and/or haloperidol. Other drugs used in treating a variety of mental disorders, inclusive future drugs, can also be tested pharmacogenomically in accordance with the teachings and the broad spirit of the present invention. Hence, the mental disorder drug can be any known antipsychotic and/or neuroleptic drug for the treatment of patients with mental disorder. Examples include, but are not limited to, thioridazine, clozapine, haloperidol, fluphenazine, cholrpromazine, risperidone, levomepromazine, perhenazine, chlorprothixene, pimozide, sulpiride, olanzapine, zuclopenthixol, amitriptyline, imipramine, clomipramine, desipramine, doxepin, mianserin, maprotiline, phenelzine, fluoxetine, trazodone, fluvoxamine, sertraline, paroxetine, reboxetine, citalopram, nefazodone, venlafoxine, lithium salts, carbamazapine, valproic acid, and clonazepam.

The mental disorder for which drug responsiveness can be determined using the methods of the present invention include, for example, schizophrenia, schizoaffective disorder, bipolar disorder, depression, obsessive compulsive disorder, panic disorder, agoraphobia, specific phobia, social phobia, post-traumatic stress disorder, pain disorder, anxiety, somatization disorder, anorexia nervosa, bulimia, and/or nervosa.

### Predisposition to Bipolar Disorder

The inventors of the present invention have uncovered that the G allele and the GG genotype of SNP rs165599, and the C allele and the CC genotype of SNP rs737865 are highly and significantly associated with bipolar disorder. Moreover, as is further demonstrated in Example 3 of the Examples section which follows and in Table 1 hereinabove, the predisposition probability to bipolar disorder in females having the GG genotype of SNP rs165599 is 104% higher than in females having the protective AA genotype. In addition, the predisposition probability to bipolar disorder in females having the CC-GG double genotype of SNPs rs737865-rs165599 is 65% higher than in females having the protective double genotype. These results suggest the use of the G allele and/or the GG genotype of SNP rs165599, and the C allele and/or the CC genotype of SNPs rs737865 and of any SNPs in linkage disequilibrium with them for determination of bipolar predisposition.

Therefore, according to still a further aspect of the present invention there is provided a method of determining a predisposition to develop bipolar disorder in a subject. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with bipolar disorder, thereby determining the predisposition of the subject of developing bipolar disorder.

According to still a further aspect of the present invention there is provided a kit for determining a predisposition to develop bipolar disorder in a subject. The kit, according to this aspect of the present invention comprises a packaging material packaging at least one reagent, the at least one reagent for determining a presence in a homozygous or heterozygous form, or an absence, of at least one genotype in the COMT locus or in neighboring loci, the neighboring loci are in linkage disequilibrium with the COMT locus, the genotype having been associated with bipolar disorder. The kit further comprises a notification in or on the packaging material, the notification identifying the kit for use in determining a predisposition of a subject of developing bipolar disorder. Preferably, the notification also provides for instructions of using the kit in determining the predisposition of a subject of developing bipolar disorder.

The G allele and/or the GG genotype of SNP rs165599, and the C allele and/or the CC genotype of SNPs rs737865 were found to be associated with bipolar disorder in females in higher association than in males.

Therefore, according to still a further aspect of the present invention there is provided a method of determining a predisposition to develop bipolar disorder in a female subject. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with bipolar disorder in females in higher association than in males, thereby determining the predisposition of the female subject of developing bipolar disorder.

According to still a further aspect of the present invention there is provided a kit for determining a predisposition to develop bipolar disorder in a female subject. The kit, according to this aspect of the present invention comprises a packaging material packaging at least one reagent, the at least one reagent for determining a presence in a homozygous or heterozygous form, or an absence, of at least one genotype in the COMT locus or in neighboring loci, the neighboring loci are in linkage disequilibrium with the COMT locus, the genotype having been associated with bipolar disorder in females in higher association than in males. The kit further comprises a notification in or on the packaging material, the notification identifying the kit for use in determining a predisposition of a female subject of developing bipolar disorder. Preferably, the notification also provides for instructions of using the kit in determining the predisposition of a female subject of developing bipolar disorder.

Genotypes in the COMT locus can also assist in the diagnosis of bipolar disorder. Thus according to still a further aspect of the present invention there is provided a method of assisting in diagnosing bipolar disorder in a subject in need thereof. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with bipolar disorder, thereby assisting in diagnosing the bipolar disorder in the subject.

According to still a further aspect of the present invention there is provided a kit for assisting in diagnosing bipolar disorder in a subject in need thereof. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with bipolar disorder. The kit further comprises a notification in or on the packaging material, the notification identifying the kit for use in assisting in diagnosing bipolar disorder in a subject. Preferably, the notification also provides for instructions of using the kit in assisting in diagnosing bipolar disorder in a subject.

According to still a further aspect of the present invention there is provided a method of assisting in diagnosing bipolar disorder in a female subject in need thereof. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with bipolar disorder in females in higher association than in males, thereby assisting in diagnosing the bipolar disorder in the female subject.

According to still a further aspect of the present invention there is provided a kit for assisting in diagnosing bipolar disorder in a female subject in need thereof. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with bipolar disorder in females in higher association than in males. The kit further comprises a notification in or on the packaging material, the notification identifying the kit for use in assisting in diagnosing bipolar disorder in a female subject. Preferably, the notification also provides for instructions of using the kit in assisting in diagnosing bipolar disorder in a female subject.

### Predisposition to Breast Cancer

Several association studies have focused on the association of the COMT gene with breast cancer. However, as is further described hereinabove, these studies were of a statistically low-power and resulted in inconclusive findings. WO2001US0021954 patent by Fritz, F. Parl, which is incorporated herein by reference, discloses an association of the low-activity COMT polymorph encoded by the A allele of SNP rs4680 with breast cancer. In sharp contrast to WO2001US0021954 patent, the inventors of the present invention conducted a comprehensive breast cancer case-control study and have found that the high-activity COMT polymorph encoded by the G allele of SNP rs4680 is significantly associated with breast cancer. Moreover, as is further demonstrated in Example 4 of the Examples section which follows and in Table 1 hereinabove, the G allele and the GG genotype of SNP rs4680, and the G allele and the GG genotype of SNP rs165599 are highly and significantly associated with breast cancer. The predisposition probabilities to develop breast cancer in individuals homozygous for these two risk alleles, i.e., the G allele of SNP rs4680 and the G allele of SNP rs165599, are 35% and 85%, respectively, higher than in individuals having the protective genotypes. In addition the T-G haplotype of SNPs rs737865-rs4680 was also found to be associated with breast cancer with increased predisposition probability of 155% in individuals homozygous for the double risk genotype over individuals homozygous for the double protective genotype. In addition, breast cancer cases with age of onset higher than 50 years as well as non-familial cases of breast cancer were significantly associated with the G alleles and the GG genotypes of SNPs rs4680 and rs165599. These results demonstrate a high association with breast cancer of SNPs in the COMT locus and of SNPs in neighboring loci such as those coding for the ARVCF protein set forth in SEQ ID NO:31 and the TXNRD2 protein forms set forth in SEQ ID NOs: 32, 33 and 34, which are in linkage disequilibrium with SNPs rs4680, rs165599, and/or rs737865, and suggest their use for determining predisposition of a subject of developing breast cancer.

Thus, according to still a further aspect of the present invention there is provided a method of determining a predisposition to develop breast cancer in a subject. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with breast cancer, thereby determining the predisposition of the subject of developing breast cancer.

According to still a further aspect of the present invention there is provided a kit for determining a predisposition to develop breast cancer in a subject. The kit, according to this aspect of the present invention comprises a packaging material packaging at least one reagent, the at least one reagent for determining a presence in a homozygous or heterozygous form, or an absence, of at least one genotype in the COMT locus or in neighboring loci, the neighboring loci are in linkage disequilibrium with the COMT locus, the genotype having been associated with breast cancer. The kit further comprises a notification in or on the packaging material, the notification identifying the kit for use in determining a predisposition of a subject of developing breast cancer. Preferably, the notification also provides for instructions of using the kit in determining a predisposition of a subject of developing breast cancer.

Genotypes of SNPs in the COMT locus can also assist in the diagnosis of breast cancer. Thus, according to still a further aspect of the present invention there is provided a method of assisting in diagnosing breast cancer in a subject in need thereof. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with breast cancer, thereby assisting in diagnosing the breast cancer in the subject.

According to still a further aspect of the present invention there is provided a kit for assisting in diagnosing breast cancer in a subject in need thereof. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with breast cancer. The kit further comprises a notification in or on the packaging material, the notification identifying the kit for use in assisting in diagnosing breast cancer in a subject. Preferably, the notification also provides for instructions of using the kit in assisting in diagnosing breast cancer in a subject.

In addition, the inventors of the present invention have uncovered a strong correlation between SNPs in the COMT locus and a high malignancy form of breast cancer which is characterized by the presence of breast cancer metastases in the lymph nodes of a breast cancer patient. More specifically, the G allele and the GG genotype of SNP rs4680 and the G allele and the GG genotype of SNP rs165599 were found to be highly and significantly associated with a more aggressive form of breast cancer. Therefore, these results teach the usefulness of these COMT genotypes and genotypes in neighboring loci for determining the prognosis of patients having breast cancer.

Thus, according to still a further aspect of the present invention there is provided a method of determining a prognosis of a breast cancer in a subject in need thereof. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with lymph node metastases of breast cancer, thereby determining the prognosis of the breast cancer in the subject.

According to still a further aspect of the present invention there is provided a kit for determining a prognosis of a breast cancer in a subject in need thereof. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with lymph node metastases of breast cancer. The kit further comprises a notification in or on the packaging material, the notification identifying the kit for use in determining a prognosis of a breast cancer in a subject. Preferably, the notification also provides for instructions of using the kit in determining the prognosis of the breast cancer in a subject.

### Predisposition to Colorectal Cancer

The inventors of the present invention have uncovered that the G allele and the GG genotype of SNP rs4680 and the G allele and the GG genotype of SNP rs165599 were highly and significantly associated with colorectal cancer. In addition, as is further shown in Example 5 of the Examples section which follows and in Table 1 hereinabove, the predisposition probabilities of developing colorectal cancer in individuals having the risk genotypes of SNPs rs4680, rs165599 and rs737865 are 37%, 39% and 39% higher than in individuals having the protective genotypes. Moreover, the inventors of the present invention have found that the G-G haplotype of SNPs rs4680-rs165599 is highly and significantly associated with colorectal cancer and individuals having the GG-GG double genotype of this haplotype have 56% higher predisposition probability of developing colorectal cancer than individuals having the protective double genotype. These results therefore teach the use of SNPs in the COMT locus and of any SNPs in linkage disequilibrium with SNPs rs4680, rs165599 and rs737865 for determining a predisposition to colorectal cancer.

Thus, according to still a further aspect of the present invention there is provided a method of determining a predisposition to develop colorectal cancer in a subject. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with colorectal cancer, thereby determining the predisposition of the subject of developing colorectal cancer.

According to still a further aspect of the present invention there is provided a kit for determining a predisposition to develop colorectal cancer in a subject. The kit, according to this aspect of the present invention comprises a packaging material packaging at least one reagent, the at least one reagent for determining a presence in a homozygous or heterozygous form, or an absence, of at least one genotype in the COMT locus or in neighboring loci, the neighboring loci are in linkage disequilibrium with the COMT locus, the genotype having been associated with colorectal cancer. The kit further comprises a notification in or on the packaging material, the notification identifying the kit for use in determining a predisposition of a subject of developing colorectal cancer. Preferably, the notification also provides for instructions of using the kit in determining the predisposition of a subject of developing colorectal cancer.

Moreover, genotypes in the COMT locus can be used for diagnosing colorectal cancer. Thus, according to still a further aspect of the present invention there is provided a method of assisting in diagnosing colorectal cancer in a subject in need thereof. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with colorectal cancer, thereby assisting in diagnosing colorectal cancer in the subject.

According to still a further aspect of the present invention there is provided a kit for assisting in diagnosing colorectal cancer in a subject in need thereof. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with colorectal cancer. The kit further comprises a notification in or on the packaging material, the notification identifying the kit for use in assisting in diagnosing colorectal cancer in a subject. Preferably, the notification also provides for instructions of using the kit in assisting in diagnosing colorectal cancer in a subject.

In addition, as is further shown in Example 5 of the Examples section which follows and in Table 1 hereinabove, the inventors of the present invention have uncovered that genotypes in the COMT locus are associated with colorectal cancer in females in higher association than in males. More specifically, the G allele and the GG genotype of SNP rs4680 and the G allele and the GG genotype of SNP rs165599 are highly and significantly associated with colorectal cancer in females. In addition, the predisposition probabilities for colorectal cancer in females having the GG genotype of SNP rs4680 and the GG genotype of SNP rs165599 are 37% and 80%, respectively, higher than in females having the protective genotypes. Moreover, the G-G haplotype of SNPs rs4680-rs165599 was highly and significantly associated with colorectal cancer in females. The predisposition probability for colorectal cancer in females having the double risk genotype (i.e., GG-GG) is 110% higher than in females having the double protective genotype. These results therefore teach the use of these genotypes in the COMT locus and of any SNPs in linkage disequilibrium with SNPs rs4680, rs165599 and rs737865 for determination of predisposition to colorectal cancer in females.

Thus, according to still a further aspect of the present invention there is provided a method of determining a predisposition to develop colorectal cancer in a female subject. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with colorectal cancer in females in higher association than in males, thereby determining the predisposition of the female subject of developing colorectal cancer.

According to still a further aspect of the present invention there is provided a kit for determining a predisposition to develop colorectal cancer in a female subject. The kit, according to this aspect of the present invention comprises a packaging material packaging at least one reagent, the at least one reagent for determining a presence in a homozygous or heterozygous form, or an absence, of at least one genotype in the COMT locus or in neighboring loci, the neighboring loci are in linkage disequilibrium with the COMT locus, the genotype having been associated with colorectal cancer in females in higher association than in males. The kit further comprises a notification in or on the packaging material, the notification identifying the kit for use in determining a predisposition of a female subject of developing colorectal cancer. Preferably, the notification also provides for instructions of using the kit in determining the predisposition of the female subject of developing colorectal cancer.

Moreover, genotypes in the COMT locus can be used for diagnosing colorectal in females. Thus, according to still a further aspect of the present invention there is provided a method of assisting in diagnosing colorectal cancer in a female subject. The method according to this aspect of the present invention comprises determining a presence in a homozygous or heterozygous form, or an absence, of at least one genotype in the COMT locus or in neighboring loci, the neighboring loci are in linkage disequilibrium with the COMT locus, the at least one genotype having been associated with colorectal cancer in females in higher association than in males, thereby assisting in diagnosing colorectal cancer in the female subject.

According to still a further aspect of the present invention there is provided a kit for assisting in diagnosing colorectal cancer in a female subject in need thereof. The kit, according to this aspect of the present invention comprises a packaging material packaging at least one reagent, the at least one reagent for determining a presence in a homozygous or heterozygous form, or an absence, of a genotype in the COMT locus or in neighboring loci, the neighboring loci are in linkage disequilibrium with the COMT locus, the genotype having been associated with colorectal cancer in females in higher association than in males. The kit further comprises a notification in or on the packaging material, the notification identifying the kit for use in assisting in diagnosing colorectal cancer in a female subject. Preferably, the notification also provides for instructions of using the kit in assisting in diagnosing colorectal cancer in a female subject.

### Novel Drug Identification

The strong associations disclosed in the present invention between genotypes in the COMT locus and various complex genetic disorders such as schizophrenia, bipolar disorder, breast cancer and colorectal cancer offers the use of the COMT protein as a target for the identification of novel drugs for treating and/or preventing these disorders.

Therefore, according to still a further aspect of the present invention there is provided a method of identifying novel drugs for treatment of schizophrenia. The method according to this aspect of the present invention comprises incubating a catechol-O-methyltransferase protein or cells expressing catechol-O-methyltransferase with potential drugs and selecting for at least one drug of the potential drugs that modulates catechol-O-methyltransferase activity or expression, thereby identifying novel drugs for treatment of schizophrenia.

According to still a further aspect of the present invention there is provided a method of treating schizophrenia. The method according to this aspect of the present invention comprises administering to a subject in need thereof a therapeutically effective amount of a drug for schizophrenia, the drug for schizophrenia having been identified using the method described hereinabove.

According to still a further aspect of the present invention there is provided a method of identifying novel drugs for treatment of bipolar disorder. The method according to this aspect of the present invention comprises incubating a catechol-O-methyltransferase protein or cells expressing catechol-O-methyltransferase with potential drugs and selecting for at least one drug of the potential drugs that modulates catechol-O-methyltransferase activity or expression, thereby identifying novel drugs for treatment of bipolar disorder.

According to still a further aspect of the present invention there is provided a method of treating bipolar disorder. The method according to this aspect of the present invention comprises administering to a subject in need thereof a therapeutically effective amount of a drug for bipolar disorder, the drug for bipolar disorder having been identified using the method described hereinabove.

According to still a further aspect of the present invention there is provided a method of identifying novel drugs for treatment of breast cancer. The method according to this aspect of the present invention comprises incubating a catechol-O-methyltransferase protein or cells expressing catechol-O-methyltransferase with potential drugs and selecting for at least one drug of the potential drugs that modulates catechol-O-methyltransferase activity or expression, thereby identifying novel drugs for treatment of breast cancer.

According to still a further aspect of the present invention there is provided a method of treating breast cancer. The method according to this aspect of the present invention comprises administering to a subject in need thereof a therapeutically effective amount of a drug for breast cancer, the drug for breast cancer having been identified using the method described hereinabove.

According to still a further aspect of the present invention there is provided a method of identifying novel drugs for treatment of colorectal cancer. The method according to this aspect of the present invention comprises incubating a catechol-O-methyltransferase protein or cells expressing catechol-O-methyltransferase with potential drugs and selecting for at least one drug of the potential drugs that modulates catechol-O-methyltransferase activity or expression, thereby identifying novel drugs for treatment of colorectal cancer.

According to still a further aspect of the present invention there is provided a method of treating colorectal cancer. The method according to this aspect of the present invention comprises administering to a subject in need thereof a therapeutically effective amount of a drug for colorectal cancer, the drug for colorectal cancer having been identified using the method described hereinabove.

The potential drug can be a peptide, a polynucleotide and/or a small molecule.

The polynucleotide used by the present invention can be an antisense oligonucleotide, an siRNA, a ribozymes and/or a DNAzyme.

The peptide used by the present invention can be a small peptide, a polypeptide and/or an antibody.

Assays of catechol-O-methyltransferase activity are well known in the art, such assays are described in, for example, Dawling, S. et al., Cancer Research 61: 6716-6722, 2001.

Assays of catechol-O-methyltransferase expression are also well known in the art. Such assays may include monitoring catechol-O-methyltransferase activity, as described above, monitoring catechol-O-methyltransferase protein levels using an anti-catechol-O-methyltransferase antibody, in assays such as, for example, in situ immunochemistry staining, Western blot, ELISA, fluorescence activated cell sorting, RIA, etc., and/or monitoring catechol-O-methyltransferase mRNA levels using any one of a plurality of molecular methods based on hybridization of nucleic acids, such as, for example, in situ hybridization, Northern blot, dot blot, RNA protection assays, etc.

### Methods of Determining Genotypes/Phenotypes

According to various preferred embodiments of the present invention, determining the various alleles of SNPs in the COMT locus is effected by any one of a variety of methods including, but not limited to, a signal amplification method, a direct detection method and detection of at least one sequence change. These methods can be employed to determine the genotype of the COMT locus in a subject. As will be explained hereinbelow, determination of the COMT genotype may also be accomplished directly by analysis of the COMT gene products, and more particularly, analysis of the Val/Met polymorphism at position 158 and/or 108 of the COMT protein set forth in SEQ ID NO:29 and/or SEQ ID NO:30, respectively.

The signal amplification method: According to various preferred embodiments of the present invention amplification of, for example, a DNA molecule or an RNA molecule is used. Signal amplification methods which might be used as part of the methods and kits of the present invention include, but are not limited to PCR, LCR (LAR), Self-Sustained Synthetic Reaction (3SR/NASBA) or a Q-Beta (Qβ) Replicase reaction.

Polymerase Chain Reaction (PCR): The polymerase chain reaction (PCR), as described in U.S. Pat. Nos. 4,683,195 and 4,683,202 to Mullis and Mullis et al., is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified.”

Ligase Chain Reaction (LCR or LAR): The ligase chain reaction [LCR; sometimes referred to as “Ligase Amplification Reaction” (LAR)] described by Barany [Proc. Natl. Acad. Sci., 88:189 (1991); Barany, PCR Methods and Applic., 1:5 (1991)] and Wu and Wallace [Genomics 4:560 (1989)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, and ligation amplify a short segment of DNA. LCR has also been used in combination with PCR to achieve enhanced detection of single-base changes [Segev, PCT Publication No. WO9001069 A1 (1990)]. However, because the four oligonucleotides used in this assay can pair to form two short ligateable fragments, there is the potential for the generation of target-independent background signal. The use of LCR for mutant screening is limited to the examination of specific nucleic acid positions.

Self-Sustained Synthetic Reaction (3SR/NASBA): The self-sustained sequence replication reaction (3SR) (Guatelli et al., Proc. Natl. Acad. Sci., 87:1874-1878, 1990), with an erratum at Proc. Natl. Acad. Sci., 87:7797, (1990) is a transcription-based in vitro amplification system (Kwok et al., Proc. Natl. Acad. Sci., 86:1173-1177, 1989) that can exponentially amplify RNA sequences at a uniform temperature. The amplified RNA can then be utilized for mutation detection (Fahy et al., PCR Meth. Appl., 1:25-33, 1991). In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ) Replicase: In this method, a probe which recognizes the sequence of interest is attached to the replicatable RNA template for Qβ replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37 degrees C.). This prevents the use of high temperature as a means of achieving specificity as in the LCR, the ligation event can be used to detect a mutation at the junction site, but not elsewhere.

A successful diagnostic method must be very specific. A straight-forward method of controlling the specificity of nucleic acid hybridization is by controlling the temperature of the reaction. While the 3SR/NASBA, and Qβ systems are all able to generate a large quantity of signal, one or more of the enzymes involved in each cannot be used at high temperature (i.e., >55° C.). Therefore the reaction temperatures cannot be raised to prevent non-specific hybridization of the probes. If probes are shortened in order to make them melt more easily at low temperatures, the likelihood of having more than one perfect match in a complex genome increases. For these reasons, PCR and LCR currently dominate the research field in detection technologies.

The basis of the amplification procedure in the PCR and LCR is the fact that the products of one cycle become usable templates in all subsequent cycles, consequently doubling the population with each cycle. The final yield of any such doubling system can be expressed as: (I+X)ⁿ=y, where “X” is the mean efficiency (percent copied in each cycle), “n” is the number of cycles, and “y” is the overall efficiency, or yield of the reaction (Mullis, PCR Methods Applic., 1:1, 1991). If every copy of a target DNA is utilized as a template in every cycle of a polymerase chain reaction, then the mean efficiency is 100%. If 20 cycles of PCR are performed, then the yield will be 2²⁰, or 1,048,576 copies of the starting material. If the reaction conditions reduce the mean efficiency to 85%, then the yield in those 20 cycles will be only 1.85²⁰, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

Also, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect (Kwok et al., Nucl. Acids Res., 18:999, 1990)

A similar 3′-mismatch strategy is used with greater effect to prevent ligation in the LCR (Barany, PCR Meth. Applic., 1:5, 1991). Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

Direct detection methods: The direct detection method according to various preferred embodiments of the present invention may be, for example, a cycling probe reaction (CPR) or a branched DNA analysis.

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Traditional methods of direct detection including Northern and Southern band RNase protection assays usually require the use of radioactivity and are not amenable to automation. Recently devised techniques have sought to eliminate the use of radioactivity and/or improve the sensitivity in automateable formats. Two examples are the “Cycling Probe Reaction” (CPR), and “Branched DNA” (bDNA).

Cycling probe reaction (CPR): The cycling probe reaction (CPR) (Duck et al., BioTech., 9:142, 1990), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

Branched DNA: Branched DNA (bDNA), described by Urdea et al., Gene 61:253-264 (1987), involves oligonucleotides with branched structures that allow each individual oligonucleotide to carry 35 to 40 labels (e.g., alkaline phosphatase enzymes). While this enhances the signal from a hybridization event, signal from non-specific binding is similarly increased.

Detection of at least one sequence change: The detection of at least one sequence change according to various preferred embodiments of the present invention may be accomplished by, for example restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis or Dideoxy fingerprinting (ddF).

The demand for tests which allow the detection of specific nucleic acid sequences and sequence changes is growing rapidly in clinical diagnostics. As nucleic acid sequence data for genes from humans and pathogenic organisms accumulates, the demand for fast, cost-effective, and easy-to-use tests for as yet mutations within specific sequences is rapidly increasing.

A handful of methods have been devised to scan nucleic acid segments for single nucleotide polymorphisms. One option is to determine the entire gene sequence of each test sample (e.g., a bacterial isolate). For sequences under approximately 600 nucleotides, this may be accomplished using amplified material (e.g., PCR reaction products). This avoids the time and expense associated with cloning the segment of interest. However, specialized equipment and highly trained personnel are required, and the method is too labor-intense and expensive to be practical and effective in the clinical setting.

In view of the difficulties associated with sequencing, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a SNP of interest happens to fall within a restriction recognition sequence, a change in the pattern of digestion can be used as a diagnostic tool (e.g., restriction fragment length polymorphism [RFLP] analysis).

SNPs have been also detected by the creation or destruction of RFLPs. SNPs are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC) (Gogos et al., Nucl. Acids Res., 18:6807-6817, 1990). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes have 4 to 6 base-pair recognition sequences, and cleave too frequently for many large-scale DNA manipulations (Eckstein and Lilley (eds.), Nucleic Acids and Molecular Biology, vol. 2, Springer-Verlag, Heidelberg, 1988). Thus, it is applicable only in a small fraction of cases, as most SNPs do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered (Barlow and Lehrach, Trends Genet., 3:167, 1987). Recently, endonucleases encoded by group I introns have been discovered that might have greater than 12 base-pair specificity (Perlman and Butow, Science 246:1106, 1989), but again, these are few in number.

Allele specific oligonucleotide (ASO): If the change is not in a recognition sequence, then allele-specific oligonucleotides (ASOs), can be designed to hybridize in proximity to the polymorphic nucleotide, such that a primer extension or ligation event can be used as the indicator of a match or a mis-match. Hybridization with radioactively labeled allelic specific oligonucleotides (ASO) also has been applied to the detection of specific SNPs (Conner et al., Proc. Natl. Acad. Sci., 80:278-282, 1983). The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes (Vogelstein et al., N. Eng. J. Med., 319:525-532, 1988; and Farr et al., Proc. Natl. Acad. Sci., 85:1629-1633, 1988), and gsp/gip oncogenes (Lyons et al., Science 249:655-659, 1990). Because of the presence of various nucleotide changes in multiple positions, the ASO method requires the use of many oligonucleotides to cover all possible oncogenic mutations.

With either of the techniques described above (i.e., RFLP and ASO), the precise location of the SNP must be known in advance of the test. That is to say, they are inapplicable when one needs to detect the presence of a SNP within a gene or sequence of interest.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of SNPs in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE (Abrams et al., Genomics 7:463-475, 1990). Attaching a GC clamp to one primer is critical to ensure that the amplified sequence has a low dissociation temperature (Sheffield et al., Proc. Natl. Acad. Sci., 86:232-236, 1989; and Lerman and Silverstein, Meth. Enzymol., 155:482-501, 1987). Modifications of the technique have been developed, using temperature gradients (Wartell et al., Nucl. Acids Res., 18:2699-2701, 1990), and the method can be also applied to RNA:RNA duplexes (Smith et al., Genomics 3:217-223, 1988).

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE) (Borrensen et al., Proc. Natl. Acad. Sci. USA 88:8405, 1991). CDGE requires that gels be performed under different denaturant conditions in order to reach high efficiency for the detection of SNPs.

A technique analogous to DGGE, termed temperature gradient gel electrophoresis (TGGE), uses a thermal gradient rather than a chemical denaturant gradient (Scholz, et al., Hum. Mol. Genet. 2:2155, 1993). TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues (reviewed by Hayashi, PCR Meth. Appl., 1:34-38, 1991) and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations (Orita, et al., Genomics 5:874-879, 1989).

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations (Liu and Sommer, PCR Methods Appli., 4:97, 1994). The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

According to a presently preferred embodiment of the present invention the step of determining the genotype of the SNPs in the COMT locus or in neighboring loci in a DNA sample of a subject is effected by the Pyrosequencing™ analysis, the Acycloprime™ analysis and RFLP as is further described in the Examples section which follows. However, alternative methods can be employed, including, but not limited to, nucleic acid sequencing, polymerase chain reaction, ligase chain reaction, self-sustained synthetic reaction, Qβ-Replicase, cycling probe reaction, branched DNA, mismatch chemical cleavage, heteroduplex analysis, allele-specific oligonucleotides, denaturing gradient gel electrophoresis, constant denaturant gel electrophoresis, temperature gradient gel electrophoresis and dideoxy fingerprinting.

Determination of the COMT Val/Met polymorphism at position 158 and/or 108 of SEQ ID NO:29 and/or SEQ ID NO:30 may be accomplished directly, by analyzing the protein gene products of the COMT gene, or portions thereof. Such a direct analysis is often accomplished using an immunological detection method.

Immunological detection methods: The immunological detection methods used in context of the present invention are fully explained in, for example, “Using Antibodies: A Laboratory Manual” (Ed Harlow, David Lane eds., Cold Spring Harbor Laboratory Press (1999)) and those familiar with the art will be capable of implementing the various techniques summarized hereinbelow as part of the present invention. All of the immunological techniques require antibodies specific to at least one of the two COMT alleles. Immunological detection methods suited for use as part of the present invention include, but are not limited to, radio-immunoassay (RIA), enzyme linked immunosorbent assay (ELISA), western blot, immunohistochemical analysis, and fluorescence activated cell sorting (FACS).

Radio-immunoassay (RIA): In one version, this method involves precipitation of the desired substrate, COMT in this case and in the methods detailed hereinbelow, with a specific antibody and radiolabelled antibody binding protein (e.g., protein A labeled with I¹²⁵) immobilized on a precipitable carrier such as agarose beads. The number of counts in the precipitated pellet is proportional to the amount of substrate. In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, calorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a calorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

It will be appreciated by one ordinarily skilled in the art that determining the COMT phenotype of an individual, either directly or genetically, may be effected using any suitable biological sample derived from the examined individual, including, but not limited to, blood, plasma, blood cells, saliva or cells derived by mouth wash, and body secretions such as urine and tears, and from biopsies, etc. Alternatively, nucleic acid tests can be performed on dry samples (e.g. hair or skin). The sample may contain genomic DNA, cDNA or RNA. Methods of preparing genomic DNA or cDNA and RNA are well known in the art. For prenatal diagnosis, fetal nucleic acid samples can be obtained from maternal blood as described in International Patent Application No. WO91/07660 to Bianchi. Alternatively, amniocytes or chorionic villi may be obtained for performing prenatal testing.

The above genotype/phenotype detection methods can be used, in context of the methods and kits of the present invention to determine a presence in a homozygous or heterozygous form, or an absence, of at least one genotype in the COMT locus or in neighboring loci, the neighboring loci are in linkage disequilibrium with the COMT locus; whereby the COMT genotype is a guanine nucleotide—and/or an adenosine nucleotide—containing allele of SNP rs4680 set forth in SEQ ID NO:5; a guanine nucleotide—and/or an adenosine nucleotide—containing allele of SNP rs165599 set forth in SEQ ID NO:9 and/or a cytosine nucleotide—and/or a thymine nucleotide—containing allele of SNP rs737865 set forth in SEQ ID NO: 1; and/or a COMT allelic haplotype which comprises at least two of the above genotypes.

### Reagents

The kits of the present invention may include any reagent needed to perform the above genotype/phenotype detection methods and or of any other genotype/phenotype detection methods not specifically mentioned. To this end, the kits of the present invention may include an oligonucleotide, an antibody and/or a DNA chip.

The oligonucleotide of the present invention can have a sequence selected differentially hybridizeable to at least one allele of SNP rs4680, rs165599, and/or rs737865, and can differentiate between polymorphs of the SNP via differential hybridization, differential template dependent primer extension reaction and/or a differential template dependent ligation reaction. The oligonucleotide of the present invention can also have a sequence selected hybridizeable adjacent to SNP rs4680, rs165599, and/or rs737865, and can be used to amplify polymorphs of the SNP via an amplification reaction.

The antibody used in the kits of the present invention is selected differentially interactable with at least one form of a COMT protein encoded by a COMT allele having an SNP rs4680, and can differentiate between polymorphs of the COMT protein via differential antibody interaction. Antibodies useful in context of this embodiment of the invention can be prepared using methods of antibody preparation well known to one of ordinary skills in the art, using, for example, synthetic peptides derived from the two different forms of the COMP protein for vaccination of antibody producing animals and subsequent isolation of antibodies therefrom. Monoclonal antibodies specific to each of the COMT variants can also be prepared as is described, for example, in “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980).

The DNA chip used in context of the present invention contains at least one oligonucleotide attached to it which has a sequence selected differentially hybridizeable to at least one allele of SNP rs4680, rs165599, and/or rs737865, and can differentiate between polymorphs of the SNP via differential hybridization.

The reagent in the kits of the present invention are designed for performing a detection method such as a signal amplification method, a direct detection method, and/or detection of at least one sequence change. As described above, the signal amplification method used by the present invention can amplify a DNA molecule and/or an RNA molecule. Alternatively, or additionally, the signal amplification method can be PCR, LCR (LAR), Self-Sustained Synthetic Reaction (3SR/NASBA), and/or Q-Beta (Qβ) Replicase reaction. The direct detection method of the present invention can be a cycling probe reaction (CPR), and/or a branched DNA analysis. The detection of the sequence is preferably employed by restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis, and/or Dideoxy fingerprinting (ddF). Alternatively, the reagents used by the kits of the present invention are designed for performing an immunological detection method for a COMT protein encoded by the COMT locus. As is further detailed hereinabove, the immunological detection method can be, for example, a radio-immunoassay (RIA), an enzyme linked immunosorbent assay (ELISA), a western blot, an immunohistochemical analysis, and/or fluorescence activated cell sorting (FACS).

The oligonucleotides used in context of the present invention can be of any length or sequence, can be DNA or RNA, or any modification thereof. It is necessary, however, that the length and sequence of the oligonucleotides be chosen to optimize the specificity of the hybridization to the target sequences of interest. Time and expense considerations tend to shift preference toward shorter oligonucleotides which are still sufficiently long to ensure high sequence specificity while at the same time rapid, easy and accurate preparation.

The oligonucleotides may be of any suitable species, preferably an oligodeoxyribonucleotide, an oligoribonucleotide, a protein nucleic acid or a copolymer of deoxyribonucleotides, protein nucleic acids and ribonucleotides. Use of other analogs is also envisaged. The oligonucleotide can be either natural or synthetic. The oligonucleotide primer can be synthesized enzymatically in vivo, enzymatically in vitro, or non-enzymatically. Solid phase synthesis is presently preferred.

In addition, the oligonucleotide must be capable of hybridizing or annealing with a stretch of nucleotide bases present in the nucleic acid of interest. One way to accomplish the desired hybridization is to have the “template dependent oligonucleotide” be substantially complementary or fully complementary to the known nucleotide bases sequence of for example the SNPs in the COMT locus.

As use herein the phrase “selected differentially hybridizeable” refers to a selective hybridization of an oligonucleotide probe to a specific allele, or stretch of DNA on and over a polymorphic site. For example, if an individual is heterozygote to the G/A polymorphism of SNP rs4680 in the COMT locus and the oligonucleotide contains a cytosine nucleotide at the position complementary to the polymorphic nucleotide, then the oligonucleotide will hybridize selectively to the guanine nucleotide—containing allele and not to the adenosine nucleotide—containing allele.

The phrase “differential template dependent primer extension reaction” refers to the use of an oligonucleotide primer complementary to a sequence adjacent to and 5′ of a polymorphic nucleotide of an SNP. Preferably, the 3′-OH of the oligonucleotide is complementary to the nucleotide next to the polymorphic site. When template dependent primer extension is used, the DNA polymerase elongates the oligonucleotide sequence according to the nucleotide present at the polymorphic site.

A DNA chip describes a flat surface which contains a large number (e.g., thousands) of unique DNA molecules (e.g., probes, oligonucleotides) immobilized on the flat surface in the form of an array (e.g., checker board). Methods of producing DNA chips include the photo-lithographic method used by Affymax (Fodor, S. P. A., et al., Science, 1991, 251, 767-773). Another approach is to use pre-synthesized molecules which are applied and immobilized to a suitable substrate (e.g., microporous membrane). DNA chips are used for example, for hybridization of an unknown sample of DNA (target) which forms a unique hybridization pattern on the DNA chip. The pattern is indicative of the strength of interaction between the target DNA and the various immobilized probes and can yield sequence information. When the sequence of the target DNA is not known the technology is generally referred to as sequencing by hybridization (SBH) as described in U.S. Pat. No. 5,202,231 which is incorporated here by reference. In other applications where the sequence of the target is known and detection is directed at identification of a change associated with a disease state the method is commonly referred to as “re-sequencing” or allele specific oligonucleotide hybridization.

### Treatment/Prevention of Disease

The Val/Met polymorphism of the COMT enzyme encoded by SNP rs4680 is a functional polymorphism which determines the level of activity (e.g., high or low, respectively) of the COMT enzyme. The present invention discloses a strong association of the high activity polymorph of the COMT enzyme exhibiting the Val amino acid at position 158 of SEQ ID NO:29 and/or at position 108 of SEQ ID NO:30 to schizophrenia, breast cancer and colorectal cancer. Therefore, the inventors of the present invention have uncovered a method of treating and/or preventing these disorders by the use of effective amounts of anti COMT agents.

Thus, according to still a further aspect of the present invention there is provided a method of treating and/or preventing schizophrenia. The method according to this aspect of the present invention comprises administering to a subject in need thereof a therapeutically effective amount of at least one agent capable of inhibiting COMT protein expression or activity.

According to still a further aspect of the present invention there is provided a method of preventing breast cancer. The method according to this aspect of the present invention comprises administering to a subject in need thereof a therapeutically prophylactically effective amount of at least one agents capable of inhibiting COMT protein expression or activity.

According to still a further aspect of the present invention there is provided a method of treating and/or preventing colorectal cancer. The method according to this aspect of the present invention comprises administering to a subject in need thereof a therapeutically effective amount of at least one agents capable of inhibiting COMT protein expression or activity.

The agent used in context of the present invention to inhibit COMT activity or expression can be an anti COMT antibody, a polynucleotide encoding an intracellular anti COMT antibody, an anti COMT antisense molecule, an anti COMT siRNA, an anti COMT ribozyme, an anti COMT DNAzyme, and/or a COMT inhibitor.

Anti COMT antibody: An anti COMT antibody can be prepared by conventional antibody preparation methods.

Antibodies of the present invention can be used for recognition of the various COMT polymorphs as well as for the inhibition of the COMT enzyme activity and/or expression.

The term “antibody” as used in the present invention includes intact molecules as well as functional fragments thereof, such as Fab, F(ab′)₂, and Fv that are capable of binding to macrophages. These functional antibody fragments are defined as follows: Fab, the fragment which contains a monovalent antigen-binding fragment of an antibody molecule, can be produced by digestion of whole antibody with the enzyme papain to yield an intact light chain and a portion of one heavy chain; Fab′, the fragment of an antibody molecule that can be obtained by treating whole antibody with pepsin, followed by reduction, to yield an intact light chain and a portion of the heavy chain; two Fab′ fragments are obtained per antibody molecule; (Fab′)₂, the fragment of the antibody that can be obtained by treating whole antibody with the enzyme pepsin without subsequent reduction; F(ab′)₂ is a dimer of two Fab′ fragments held together by two disulfide bonds; Fv, defined as a genetically engineered fragment containing the variable region of the light chain and the variable region of the heavy chain expressed as two chains; and single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art. See for example, Harlow and Lane, Antibodies: A Laboratory Manual, Cold Spring Harbor Laboratory, New York, 1988, incorporated herein by reference.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of V_{H} and V_{L} chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as gluteraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boemer et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

Intracellular anti COMT antibody: The polynucleotide encoding an anti COMT antibody, obtained as described above, is inserted into a suitable mammalian expression vector dedicated for intracellular expression of a protein encoded thereby.

To generate such an expression vector, a polynucleotide segment encoding a anti COMT antibody, devoid of extracellular secretion signal peptide sequence, is ligated into, for example, a commercially available expression vector system suitable for transforming mammalian cells and for directing the expression of the anti COMT antibody within the transformed cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter polypeptides, etc.

Suitable mammalian expression vectors for use with the present invention include, but are not limited to, pcDNA3, pcDNA3.1(+/−), pZeoSV2(+/−), pSecTag2, pDisplay, pEF/myc/cyto, pCMV/myc/cyto, pCR3.1, which are available from Invitrogen, pCI which is available from Promega, pBK-RSV and pBK-CMV which are available from Strategene, pTRES which is available from Clontech, and their derivatives.

Viral expression vectors can be particularly useful for introducing an anti COMT antibody polynucleotide into a cell. The host cell can be a cell in a subject, a cell in vivo, or a cell ex vivo (see, for example U.S. Pat. No. 5,399,346). Viral vectors provide the advantage that they can infect host cells with relatively high efficiency and can infect specific cell types. Viral vectors can be particularly useful for introducing a polynucleotide useful in performing a method of the invention into a target cell. Viral vectors have been developed for use in particular host systems, particularly mammalian systems and include, for example, retroviral vectors, other lentivirus vectors such as those based on the human immunodeficiency virus (HIV), adenovirus vectors, adeno-associated virus vectors, herpes virus vectors, vaccinia virus vectors, and the like (see Miller and Rosman, BioTechniques 7:980-990, 1992; Anderson et al., Nature 392:25-30 Suppl., 1998; Verma and Somia, Nature 389:239-242, 1997; Wilson, New Engl. J. Med. 334:1185-1187(1996)).

When retroviruses, for example, are used for polynucleotide transfer, replication competent retroviruses theoretically can develop due to recombination of retroviral vector and viral gene sequences in the packaging cell line utilized to produce the retroviral vector. Packaging cell lines in which the production of replication competent virus by recombination has been reduced or eliminated can be used to minimize the likelihood that a replication competent retrovirus will be produced. All retroviral vector supernatants used to infect cells are screened for replication competent virus by standard assays such as PCR and reverse transcriptase assays. Retroviral vectors allow for integration of a heterologous gene into a host cell genome, which allows for the gene to be passed to daughter cells following cell division.

Mammalian cell systems which utilize recombinant viruses or viral elements to direct expression can be engineered. For example, when using adenovirus expression vectors, the anti COMT protein coding sequence can be ligated to an adenovirus transcription/translation control complex, e.g., the late promoter and tripartite leader sequence. Alternatively, the vaccinia virus 7.5K promoter can be used (Mackett et al., Proc. Natl. Acad. Sci., USA 79:7415-7419, 1982; Mackett et al, J. Virol. 49:857-864, 1984; Panicali et al., Proc. Natl. Acad. Sci., USA 79:4927-4931, 1982). Particularly useful are bovine papilloma virus vectors, which can replicate as extrachromosomal elements (Sarver et al., Mol. Cell. Biol. 1:486, 1981). Shortly after entry of this DNA into mouse cells, the plasmid replicates to about 100 to 200 copies per cell. Transcription of the inserted cDNA yielding a high level of expression may result without integration of the plasmid into the host cell chromosome. These vectors can be used for stable expression by including a selectable marker in the plasmid, such as, for example, the neo gene. Alternatively, the retroviral genome can be modified for use as a vector capable of introducing and directing the expression of an anti COMT protein in the host cells (Cone and Mulligan, Proc. Natl. Acad. Sci., USA 81:6349-6353, 1984). High level expression can also be achieved using inducible promoters, including, but not limited to, the metallothionein IIA promoter and heat shock promoters.

The expression vector described above can be delivered into cells using a variety of delivery approaches, including, but not limited to, microinjection, electroporation, liposomes, epidermal patches, iontophoresis or receptor-mediated endocytosis. The selection of a particular method will depend, for example, on the cell into which the polynucleotide is to be introduced, as well as whether the cell is isolated in culture, or is in a tissue or organ in culture or in situ.

Anti COMT antisense oligonucleotides/vectors: In human gene therapy, antisense nucleic acid technology has been one of the major tools of choice to inactivate genes where expression causes disease and is thus undesirable [Walton et al., 1999, Biotechnol Bioeng 65(1):1-9; Matveeva et al., 1998, Nature Biotechnology 16, 1374-1375].

By forming a DNA/target mRNA heteroduplex, the anti-sense molecule passively facilitates cleavage and degradation of the target mRNA component by endogenous RNAse H enzyme.

An antisense molecule which can be used in context of the present invention includes a polynucleotide or a polynucleotide analog of at least 10 bases, preferably between 10 and 15, more preferably between 50 and 20 bases, most preferably, at least 17, at least 18, at least 19, at least 20, at least 22, at least 25, at least 30 or at least 40 bases which is hybridizeable in vivo with its target molecules.

The antisense oligonucleotides used by the present invention can be expressed from nucleic acid construct administered into the tissue, in which case constitutive or preferably inducible promoters are preferably used such that antisense expression can be switched on and off, or alternatively such oligonucleotides can be chemically synthesized and administered directly into the tissue, as part of, for example, a pharmaceutical composition.

The ability of chemically synthesizing oligonucleotides and analogs thereof having a selected predetermined sequence offers means for downmodulating gene expression. Four types of gene expression modulation strategies may be considered.

At the transcription level, antisense or sense oligonucleotides or analogs that bind to the genomic DNA by strand displacement or the formation of a triple helix, may prevent transcription. At the transcript level, antisense oligonucleotides or analogs that bind target mRNA molecules lead to the enzymatic cleavage of the hybrid by intracellular RNase H. In this case, by hybridizing to the targeted mRNA, the oligonucleotides or oligonucleotide analogs provide a duplex hybrid recognized and destroyed by the RNase H enzyme. Alternatively, such hybrid formation may lead to interference with correct splicing. As a result, in both cases, the number of the target mRNA intact transcripts ready for translation is reduced or eliminated.

At the translation level, antisense oligonucleotides or analogs that bind target mRNA molecules prevent, by steric hindrance, binding of essential translation factors (ribosomes), to the target mRNA, a phenomenon known in the art as hybridization arrest, disabling the translation of such mRNAs.

Several prior art studies have shown that antisense oligonucleotides can be effective in vivo. For example, antisense molecules have been used to arrest hematopoietic cell proliferation (Szczylik et al., Science 253: 562 1991), growth (Calabretta et al. Proc. Natl. Acad. Sci. USA 88:2351, 1991), or entry into the S phase of the cell cycle (Heikhila et al., Nature, 328:445, 1987) and to prevent receptor mediated responses (Burch and Mahan, J. Clin. Invest. 88:1190, 1991).

Several considerations must be taken into account when designing antisense oligonucleotides. For efficient in vivo inhibition of gene expression using antisense oligonucleotides or analogs, the oligonucleotides or analogs must fulfill the following requirements (i) sufficient specificity in binding to the target sequence; (ii) solubility in water; (iii) stability against intra- and extracellular nucleases; (iv) capability of penetration through the cell membrane; and (v) when used to treat an organism, low toxicity. The binding affinity of an antisense oligonucleotide can be predicted (Walton et al., 1999, Biotechnol Bioeng 65: 1-9; Matveeva et al., 1998, Nature Biotechnology 16, 1374-1375.)

Unmodified oligonucleotides are typically impractical for use as antisense sequences since they have short in vivo half-lives, during which they are degraded rapidly by nucleases. Furthermore, they are difficult to prepare in more than milligram quantities. In addition, such oligonucleotides are poor cell membrane penetrants.

Thus it is apparent that in order to meet all the above listed requirements, oligonucleotide analogs need to be devised in a suitable manner.

For example, problems arising in connection with double-stranded DNA (dsDNA) recognition through triple helix formation have been diminished by a clever “switch back” chemical linking, whereby a sequence of polypurine on one strand is recognized, and by “switching back”, a homopurine sequence on the other strand can be recognized. Also, good helix formation has been obtained by using artificial bases, thereby improving binding conditions with regard to ionic strength and pH.

In addition, in order to improve half-life as well as membrane penetration, a large number of variations in polynucleotide backbones have been done, nevertheless with little success.

Oligonucleotides can be modified either in the base, the sugar or the phosphate moiety. These modifications include, for example, the use of methylphosphonates, monothiophosphates, dithiophosphates, phosphoramidates, phosphate esters, bridged phosphorothioates, bridged phosphoramidates, bridged methylenephosphonates, dephospho internucleotide analogs with siloxane bridges, carbonate bridges, carboxymethyl ester bridges, carbonate bridges, carboxymethyl ester bridges, acetamide bridges, carbamate bridges, thioether bridges, sulfoxy bridges, sulfono bridges, various “plastic” DNAs, anomeric bridges and borane derivatives (Cook, 1991, Anti-Cancer Drug Design 6:585).

International patent application WO 89/12060 discloses various building blocks for synthesizing oligonucleotide analogs, as well as oligonucleotide analogs formed by joining such building blocks in a defined sequence. The building blocks may be either “rigid” (i.e., containing a ring structure) or “flexible” (i.e., lacking a ring structure). In both cases, the building blocks contain a hydroxy group and a mercapto group, through which the building blocks are said to join to form oligonucleotide analogs. The linking moiety in the oligonucleotide analogs is selected from the group consisting of sulfide (—S—), sulfoxide (—SO—), and sulfone (—SO2-).

International patent application WO 92/20702 describes an acyclic oligonucleotide which includes a peptide backbone on which any selected chemical nucleobases or analogs are stringed and serve as coding characters as they do in natural DNA or RNA. These new compounds, known as peptide nucleic acids (PNAs), are not only more stable in cells than their natural counterparts, but also bind natural DNA and RNA 50 to 100 times more tightly than the natural nucleic acids cling to each other. PNA oligomers can be synthesized from the four protected monomers containing thymine, cytosine, adenine and guanine by Merrifield solid-phase peptide synthesis. In order to increase solubility in water and to prevent aggregation, a lysine amide group is placed at the C-terminal region.

Thus, antisense technology requires pairing of messenger RNA with an oligonucleotide to form a double helix that inhibits translation. The concept of antisense-mediated gene therapy was already introduced in 1978 for cancer therapy. This approach was based on certain genes that are crucial in cell division and growth of cancer cells. Synthetic fragments of genetic substance DNA can achieve this goal. Such molecules bind to the targeted gene molecules in RNA of tumor cells, thereby inhibiting the translation of the genes and resulting in dysfunctional growth of these cells. Other mechanisms have also been proposed. These strategies have been used, with some success in treatment of cancers, as well as other illnesses, including viral and other infectious diseases.

Antisense oligonucleotides are typically synthesized in lengths of 13-30 nucleotides. The life span of oligonucleotide molecules in blood is rather short. Thus, they have to be chemically modified to prevent destruction by ubiquitous nucleases present in the body. Phosphorothioates are very widely used modification in antisense oligonucleotide ongoing clinical trials. A new generation of antisense molecules consist of hybrid antisense oligonucleotide with a central portion of synthetic DNA while four bases on each end have been modified with 2′O-methyl ribose to resemble RNA. In preclinical studies in laboratory animals, such compounds have demonstrated greater stability to metabolism in body tissues and an improved safety profile when compared with the first-generation unmodified phosphorothioate. Dozens of other nucleotide analogs have also been tested in antisense technology.

RNA oligonucleotides may also be used for antisense inhibition as they form a stable RNA-RNA duplex with the target, suggesting efficient inhibition. However, due to their low stability RNA oligonucleotides are typically expressed inside the cells using vectors designed for this purpose. This approach is favored when attempting to target a mRNA that encodes an abundant and long-lived protein.

Recent scientific publications have validated the efficacy of antisense compounds in animal models of hepatitis, cancers, coronary artery restenosis and other diseases. The first antisense drug was recently approved by the FDA. The drug, Fomivirsen, was developed by Isis, and is indicated for local treatment of cytomegalovirus in patients with A/DS who are intolerant of or have a contraindication to other treatments for CMV retinitis or who were insufficiently responsive to previous treatments for CMV retinitis (Pharmacotherapy News Network).

Several antisense compounds are now in clinical trials in the United States. These include locally administered antivirals, systemic cancer therapeutics. Antisense therapeutics has the potential to treat many life-threatening diseases with a number of advantages over traditional drugs. Traditional drugs intervene after a disease-causing protein is formed. Antisense therapeutics, however, block mRNA transcription/translation and intervene before a protein is formed, and since antisense therapeutics target only one specific mRNA, they should be more effective with fewer side effects than current protein-inhibiting therapy.

A second option for disrupting gene expression at the level of transcription uses synthetic oligonucleotides capable of hybridizing with double stranded DNA. A triple helix is formed. Such oligonucleotides may prevent binding of transcription factors to the gene's promoter and therefore inhibit transcription. Alternatively, they may prevent duplex unwinding and, therefore, transcription of genes within the triple helical structure.

Anti COMT siRNA: Another mechanism of down regulating enzymes at the transcript level is RNA interference (RNAi), an approach which utilizes small interfering dsRNA (siRNA) molecules that are homologous to the target mRNA and lead to its degradation [Carthew, 2001, Curr Opin Cell Biol 13(2):244-8]. RNAi is an evolutionarily conserved surveillance mechanism that responds to double-stranded RNA by sequence-specific silencing of homologous genes (Fire et al., 1998, Nature 391, 806-811; Zamore et al., 2000, Cell 101, 25-33). RNAi is initiated by the dsRNA-specific endonuclease dicer, which promotes cleavage of long dsRNA into double-stranded fragments between 21 and 25 nucleotides long, termed small interfering RNA (siRNAs) (Zamore et al., 2000, Cell 101, 25-33; Elbashir et al., 2001, Genes Dev. 15, 188-200; Hammond et al., 2000, Nature 404, 293-296; Bernstein et al., 2001, Nature 409, 363-366). siRNA are incorporated into a protein complex that recognizes and cleaves target mRNAs (Nykanen et al., 2001, Cell 107, 309-321).

RNAi has been increasingly used for the sequence-specific inhibition of gene expression. The possibility of interfering with any specific target RNA has rendered RNAi a valuable tool in both basic research and therapeutic applications. RNAi was first used for gene silencing in nematodes (Fire et al., 1998, Nature 391, 806-811).

Recent scientific publications have validated the efficacy of such short double stranded RNA molecules in inhibiting target mRNA expression and thus have clearly demonstrated the therapeutic potential of such molecules. For example, RNAi has been utilized to inhibit expression of hepatitis C (McCaffrey et al., 2002, Nature 418, 38-39), HIV-1 (Jacque et al., 2002, Nature 418, 435438), cervical cancer cells (Jiang and Milner 2002, Oncogene 21, 6041-8) and leukemic cells (Wilda et al., 2002, Oncogene 21, 5716-24).

Several considerations must be taken into account when designing RNAi for in vivo administration into mammalian cells. Since the introduction of dsRNA into mammalian cells does not result in efficient Dicer-mediated generation of siRNA (Caplen et al., 2000, Gene 252, 95-105; Ui-Tei et al., 2000, FEBS Lett. 479, 79-82) short siRNA duplexes of typically 21 to 25-base pairs are utilized to initiate target cleavage.

Such siRNA molecules can be chemically synthesized as 21 to 25-nucleotide siRNA duplexes (Elbashir et al., 2001, Genes Dev. 15, 188-200; McCaffrey et al., 2002, Nature 418, 38-39). Synthetic siRNA oligonucleotide duplexes can be prepared with either ribonucleotide 3′ overhangs or with deoxyribonucleotide 3′ overhangs (Hohjoh 2002, FEBS Lett. 521, 195-9). They can also be prepared as a sense-stranded DNA/antisense-stranded RNA hybrids or vise versa.

The siRNA used by the present invention can be transcribed in vitro from plasmids and administered into the tissue. Transcripts that include two self-complementary siRNAs annealed to form a loop region can be further processed by single-stranded ribonucleases and/or other proteins into a functional duplex siRNA molecule (Leirdal and Sioud, 2002, Biochem Biophys Res Commun 295, 744-8). siRNA can also be prepared from dsRNA by Escherichia coli RNase III cleavage into endoribonuclease-prepared siRNA (esiRNA).

Since approaches for introducing synthetic siRNA into cells by lipofection can result in low transfection efficiencies in some cell types and/or short-term persistence of silencing effects, vector mediated methods have been developed.

Thus, siRNA molecules utilized by the present invention are preferably delivered into cell using retroviruses. Delivery of siRNA using retroviruses provides several advantages over methods, such as lipofection, since retroviral delivery is more efficient, uniform and immediately selects for stable “knock-down” cells.

Thus, siRNA molecules of the present invention are preferably transcribed from expression vectors which can facilitate stable expression of the siRNA transcripts once introduced into a host cell. These vectors are engineered to express small hairpin RNAs (shRNAs), which are processed in vivo into siRNA molecules capable of carrying out gene-specific silencing [Brummelkamp, T. R., et al., (2002) Science 296: 550-53; Paddison, P. J., et al., (2002) Genes Dev. 16:948-58; Paul et al. (2002) Nature Biotech. 20: 505-08, Yu, J. Y et al., (2002) Proc. Natl. Acad. Sci. USA 99: 6047-52].

An example of a suitable expression vector is the pSUPER™, which includes the polymerase-III H1-RNA gene promoter with a well defined start of transcription and a termination signal consisting of five thymidines in a row (T5) (Brummelkamp, 2002, Science 296: 550-53). Most importantly, the cleavage of the transcript at the termination site is at a site following the second uridine, thus yielding a transcript which resembles the ends of synthetic siRNAs, which also contain nucleotide overhangs. siRNA is cloned such that it includes the sequence of interest, i.e., COMT separated by a short spacer from the reverse complement of the same sequence. The resulting transcript folds back on itself to form a stem-loop structure, which mediates COMT RNAi.

Another suitable siRNA expression vector encodes the sense and antisense siRNA under the regulation of separate polIII promoters (Miyagishi and Taira (2002) Nature Biotech. 20:497-500). The siRNA, generated by this vector also includes a 5 thymidine termination signal.

As an alternative to anti-sense molecules, catalytic nucleic acid molecules have shown promise as therapeutic agents for suppressing gene expression, and are widely discussed in the literature (Haseloff, J. & Gerlach, W. A. Nature 1988;334: 585; Breaker, R. R. and Joyce, G. Chemistry and Biology 1994; 1:223; Koizumi, M., et al. Nucleic Acids Research 1989;17:7059; Otsuka, E. and Koizumi, M., Japanese Patent No. 4,235,919; Kashani-Sabet, M., et al. Antisense Research and Development 1992;2:3-15; Raillard, S. A. and Joyce, G. F. Biochemistry 1996;35:11693; and Carmi, N. et al. Chemistry and Biology 1996;3:1039). Unlike conventional anti-sense inhibition, a catalytic nucleic acid molecule functions by binding to and actually cleaving its target mRNA. Cleavage of the target sequence depends on complementation of the target with the hybridizing regions of the catalytic nucleic acid, and the presence of a specific cleavage sequence.

The term enzymatic nucleic acid is used interchangeably with phrases such as ribozymes, catalytic RNA, enzymatic RNA, catalytic DNA, nucleozyme, DNAzyme, RNA enzyme, endoribonuclease, minizyme, leadzyme, oligozyrne or DNA enzyme, as used in the art. All of these terminologies describe nucleic acid molecules with enzymatic activity.

Anti COMT ribozymes: Ribozymes are being increasingly used for the sequence-specific inhibition of gene expression by the cleavage of mRNAs encoding proteins of interest. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders. Most notably, several ribozyme gene therapy protocols for HIV patients are already in Phase 1 trials (Welch et al., 1998, Curr. Opin. Biotechnol., 9:486-496). More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated). As described above, novel ribozymes can be designed to cleave known substrate, using either random variants of a known ribozyme or random-sequence RNA as a starting point (Pan, T. and Uhlenbeck, O. C. Biochemistry 1996;31:3887; Tsang, J. and Joyce, G. F. Biochemistry 1994;33:5966; Breaker, R. R. and Joyce, G. Chemistry and Biology 1994; 1:223). However, ribozymes may be susceptible to hydrolysis within the cells, sometimes limiting their pharmaceutical applications.

Anti COMT DNAzymes: Recently, a new class of catalytic molecules called “DNAzymes” was created (Breaker, R. R. and Joyce, G. Chemistry and Biology 1995;2:655; Santoro, S. W. & Joyce, G. F. Proc. Natl, Acad. Sci. USA 1997;943:4262). DNAzymes are single-stranded, and cleave both RNA. A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, LM Curr Opin Mol Ther 2002;4:119-21).

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Ther www.asgt.org.). In another application, DNAzymes complementary to bcr-abl oncogenes were successful in inhibiting the oncogenes expression in leukemia cells, and lessening relapse rates in autologous bone marrow transplant in cases of CML and ALL.

COMT inhibitors: The COMT inhibitor used in context of the present invention can be any compound exerting an inhibitory effect on the COMT activity and/or expression. Examples include but are not limited to 2′-fluoro-3,4-dihydroxy-5-nitrobenzophenone, 3,4-dihydroxy-5-nitrophenyl derivatives, catechol derivatives, 3,4-dihydroxy4′-methyl-5-nitrobenzophenone.

The most potent COMT inhibitor thus far reported, 3,4-dihydroxy4′-methyl-5-nitrobenzophenone (tolcapone, Australian Pat. AU-B-69764/87), and (E)-2-cyano-N,N-diethyl-3-(3,4-dihydroxy-5-nitrophenyl)acrylamide (entacapone, German Pat. DE 3740383 A 1) have inhibition constants in the low nM range. Tolcapone differs from entacapone in being a more potent inhibitor of COMT in the periphery and furthermore at penetrating into the brain to inhibit brain COMT as well.

The 3,4-dihydroxy-5-nitrophenyl derivatives used as COMT inhibitors by the present invention include entacapone (2-cyano-3-(3,4-dihydroxy-5-nitrophenyl)-N,N-diethyl-2-propenamide), nitecapone [3-(3,4-dihydroxy-5-nitrophenyl)methylene-2,4-pentanedione], N,N-diethyl-2-cyano-3-(3,4-dihydroxy-5-nitrophenyl) acrylamide, 1-[3,4-dihydroxy-5-nitrophenyl]-3-(N-3′trifluormethylphenyl)-piperazine-1-propanone dihydrochloride, 1-(3,4-dihydroxy-5-nitrophenyl)-2-phenyl-ethanone and substituted 2-phenyl-1-(3,4-dihydroxy-5-nitrophenyl)-1-ethanones derivatives as described in U.S. Pat. No. 6,512,136, which is incorporated herein by reference.

The catechol derivatives used as COMT inhibitors include, but are not limited to, those described in U.S. Pat. Nos. 6,150,412 and 5,446,194, both are incorporated herein by reference. For example, the catechol derivative can be 3,5-dinitro-catechol, 2,6-dinitro-3-hydroxy-4-methoxyphenyl derivatives and 5-hydroxy-4-methoxy-2-nitrophenyl derivatives, 2,6-diformyl-3-hydroxy-4-methoxyphenyl) derivatives, 2,6-dicyano-3-hydroxy-4-methoxyphenyl derivatives, 3,4-dihydroxy-2,6-dinitrophenylacetic acid, 3,4-dihydroxy-2,6-dinitrophenyl derivatives, 2,6-diformyl-3,4-dihydroxyphenyl derivatives, 3,4-dihydroxy-2,6-dicyanophenyl derivatives, and 4,5-dimethoxy-2-nitrophenyl derivatives.

Other COMT inhibitors have the general formula described in U.S. Pat. No. 5,112,861, which is incorporated herein by reference. Hence, additional examples of compound which can be used as COMT inhibitors in the methods of treatment of the present invention and which correspond to the general formula described in U.S. Pat. No. 5,112,861, include, but are not limited to 3-Nitro-5-[2-(4-pyridyl)vinyl]catechol, 3-nitro-5-[2-(4-quinolyl)vinyl]catechol, 4-hydroxy-3-methoxy-5-nitrocinnamic acid, 3,4-dihydroxy-5,omega-dinitrostyrene, 3,4-dihydroxy-5-nitro-omega, omega-dicyanostyrene, 3-(3,4-diacetoxy-5-nitrophenyl)-1-phenylprop-2-en-1-one, 3-(3,4-dibenzoyloxy-5-nitrophenyl)-1-phenylprop-2-en-1-one, 3-(3-pivaloyloxy-4-hydroxy-5-nitrophenyl)-1-phenyl-prop-2-en-1-one, 7-(3,4-Dihydroxy-5-nitrobenzylidene)-8-ketononanoic acid, 4′-hydroxy-3′-methoxy-5′-nitroacetophenone, 3′4′-dihydroxy-5′-nitroacetophenone.

Additional COMT inhibitors are described in Mannisto and Kaakkola, 1999 [Catechol-O-methyltransferase (COMT): biochemistry, molecular biology, pharmacology, and clinical efficacy of the new selective COMT inhibitors. Pharmacological Reviews, 51: 593-628], which is fully incorporated here by reference.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); “Approaches to Gene Mapping in Complex Human Diseases” Jonathan L. Haines and Margaret A. Pericak-Vance eds., Wiley-Liss (1998); “Genetic Dissection of Complex Traits” D.C. Rao and Michael A. Province eds., Academic Press (1999); “Introduction to Quantitative Genetics” D. S. Falconer and Trudy F. C. Mackay, Addison Wesley Longman Limited (1996); all of which are incorporated by reference as if fully set forth herein. Other general references are provided throughout this document. The procedures therein are believed to be well known in the art and are provided for the convenience of the reader. All the information contained therein is incorporated herein by reference.

General Materials and Experimental Methods

Study subjects—Study subjects were Ashkenazi Jewish with known ethnic origin of at least two generations. Subjects were recruited to study upon signing an informed consent form with approved Institutional Review Board (IRB) protocols.

Schizophrenic and bipolar disorder cases—Diagnosis included a direct interview using structured clinical interview for personality disorder (SCID), a questionnaire with inclusion and exclusion criteria and cross-references to medical records. The inclusion criteria specified diagnosis according to the Diagnostic and Statistical Manual of Mental Disorders (DSM-IV). Questionnaires also included information regarding drugs taken and their effects on the patient.

Breast cancer and colorectal cancer cases—Diagnosis included clinical and pathological findings and was performed by the caring physicians.

Control individuals—Healthy Jewish Ashkenazi individuals.

Sample preparation—Genomic DNA was prepared from peripheral blood samples using the Nucleon™ kit (Pharmacia, North Peapack, N.J., USA) according to manufacturer's instructions. DNA was diluted to 100 ng/μl and equal aliquots of DNA samples were pooled together according to disease classification or were used for individual genotyping. Prior to genotyping reactions pool and individual DNA samples were diluted to 10 and 2 ng/μl, respectively.

SNP selection—SNPs were selected from the National Center for Biotechnology Information (NCBI) SNP data base (www.ncbi.nlm.nih.gov/entrez/query.fcgi?db=snp). Preferred SNPs were potentially functional (e.g., non-synonymous, SNPs in regulatory regions) and had records of being validated on genomic DNA samples. SNPs were relatively equally spaced throughout the genomic sequence to reach maximal coverage of the gene. SNPs were analyzed by the Pyrosequencing software (Pyrosequencing™, Uppsala, Sweden) prior to primer design.

In the COMT locus the analyzed SNPs were rs737865 (SEQ ID NO:1) and rs6269 (SEQ ID NO:13) which are located in the 5′ untranslated region (UTR) of the COMT gene, rs4680 (SEQ ID NO:5) which is the known COMT (V158M of SEQ ID NO:29 or V108M of SEQ ID NO:30) polymorphism [HGVbase (http://hgvbase.cgb.ki.se/; SNP ID: SNP000000140)], rs165599 (SEQ ID NO:9) which is located 251 bp downstream of the 3′ UTR of the COMT gene, rs4633 (SEQ ID NO:17) which is the C/T-His62 COMT synonymous polymorphism, rs362204/rs3838146 (SEQ ID NO:21) which is located in the 3′ UTR of the COMT gene, rs202017 (SEQ ID NO:24), SNPs 2 and 3 [Saito, S., A. Iida, et al. (2001). Identification of 197 genetic variations in six human methyltranferase genes in the Japanese population. J Hum Genet 46(9): 529-37, SEQ ID NOs:28 and 27, respectively] which are located in the promoter region of the COMT gene.

TABLE 2
SNPs and primers from the COMT locus

| SNP | | |
|---|---|---|
| SEQ | | |
| ID | SNP ID# | Primer SEQ ID and sequence 5′→3′: |
| NO:1 | rs737865 | NO:2 (F-B): TAGTGTCTCACTGGGCTCTGC |
| | | NO:3 (R) ACCTGCTTTTTGGATTTTTCC |
| | | NO:4 (P) TTTTCCAGCCAGGG |
| NO:5 | rs4680 | NO:6 (F) TCATCACCATCGAGATCAACC |
| | | NO:7 (R-B) CCCTTTTTCCAGGTCTGACA |
| | | NO:8 (P) GGTGGATTTCGCTGG |
| NO:9 | rs165599 | NO:10 (F-B) CACAGTGGTGCAGAGGTCAG |
| | | NO:11 (R) CTGGCTGACTCCTCTTCGTTT |
| | | NO:12 (P) TTCGTTTCCCAGGC |
| NO:13 | rs6269 | NO:14 (F-B) ATTTCTGAACCTTGCCCCTCT |
| | | NO:15 (R) CAGTGCTCTGTGCTCCTCCT |
| | | NO:16 (P) TCGCCCCCTTGTG |
| NO:17 | rs4633 | NO:18 (F) CATGGGTGACACCAAGGAG |
| | | NO:19 (R-B) CTGCTCGCAGTAGGTGTCAAT |
| | | NO:20 (P) CGCATCCTGAACCA |
| NO:21 | rs362204/ | NO:22 (F) GTTCTCTGGGACCTCTGAC |
| | rs3838146 | NO:23 (R) CTGGCTGACTCCTCTTCGTTT |
| NO:24 | rs2020917 | NO:25 (F) GCTATTGCCGTGTCTGGACT |
| | | NO:26 (R) CTGACTGCACCTGTGGTCTC |
| NO:27 | SNP 3 (Saito | NO:25 (F) GCTATTGCCGTGTCTGGACT |
| | et al., 2001*) | NO:26 (R) CTGACTGCACCTGTGGTCTC |
| NO:28 | SNP 2 (Saito | NO:25 (F) GCTATTGCCGTGTCTGGACT |
| | et al., 2001*) | NO:26 (R) CTGACTGCACCTGTGGTCTC |

SNP and primers from the COMT locus. F = forward primer, R = reverse primer, B = biotinylated at 5′, P = pyro primer. *Saito, S., A. Iida, et al. (2001). Identification of 197 genetic variations in six human methyltranferase genes in the Japanese population. J Hum Genet 46(9): 529-37

Pyrosequencing analysis—PCR primers flanking the SNP were selected using the Primer3 software (http://www.genome.wi.mit.edu) to yield an average PCR product of 150 base pairs (bp). For an avidin-based magnetic bead separation of the PCR product, one of the flanking primers was 5′-conjugated to a biotin molecule. PCR reactions included 45 cycles of denaturation (20 seconds at 94° C.), annealing (30 seconds at 55° C.) and elongation (30 seconds at 72° C.) using the HotStar Taq Polymerase kit (Qiagen, Valencia, Calif., USA) according to manufacturer's instructions. Pool DNA PCR products were subsequently subjected to Pyrosequencing analysis using the Pyrosequencing specific internal primers on the PSQ™ 96 System (Pyrosequencing™, Uppsala, Sweden) according to manufacturer's protocol.

Restriction analysis—PCR products of SNPs rs4680, rs165599 and rs737865 were digested using the Nla-III, Msp-I and Bsl I restriction enzymes (both from NEBiolabs, MA, USA) respectively, and were further analyzed on 3% agarose (BMA, ME, USA) gels.

Acycloprime analysis—PCR products were analyzed using the AcycloPrime-FP SNP Detection Kit (PerkinElmer, MA, USA) on the Ultra Multi Detection Microplate Reader (Tecan; Austria) according to manufacturer's instructions.

Statistical Analysis

Linkage disequilibrium estimates—To measure the linkage disequilibrium (LD) between SNPs, haplotype frequencies were estimated using the expectation maximization (EM) [Slatkin M, Excoffier L. (1996). Testing for linkage disequilibrium in genotypic data using the Expectation-Maximization algorithm. Heredity. 76: 377-83] algorithm on 70-90 individual genotypes and the Lewontin's (D′) and Pearson correlation (r) were calculated.

Test for association—Association of a specific SNP to a disease was evaluated in a case-control study by comparing the frequency of a specific allele and/or genotype in the case population (i.e., patients) with those of the control population, using standard χ² and proportion tests under normal approximation.

Odds ratios—is an estimate of the relative risk, i.e., the increased probability of disease in populations exposed to the risk allele. OR and approximate confidence intervals, were computed in a standard way [Alan Agresti (1990). Categorical data analysis. New York: Wiley, pp. 54-55] in order to examine the structure and strength of association between genotype and disease.

Genotype relative risk (GRR)—is the increased chance of an individual with a particular genotype to develop the disease. Thus, the GRR of the risk genotype G, with respect to the protective genotype G₀, is the ratio between the risk of an individual carrying genotype G to develop the disease, and the risk of an individual carrying genotype G₀ to develop the disease. The GRR used herein is represented in terms of an appropriate odds ratio (OR) of G versus G₀ in cases and controls. Computation of the GRR of the haplotypes was based on a multiplicative model in which the GRR of an homozygote individual was the square of the GRR of an heterozygote individual. For further details see Risch and Merikangas, 1996 [The future of genetic studies of complex human diseases. Science 273: 1516-1517].

Population attributable risk (PAR)—is the percentage of cases that would not have been affected if the population was monomorphic for the protective allele and genotype. The PAR value of a certain allele is calculated by the following equation: (K-1)/K, wherein K is Σfᵢ·gᵢ, fᵢ is the frequency of the i genotype or double genotype and gᵢ is the estimated GRR of the i genotype or double genotype, respectively.

Association of haplotypes with increased risk for a disease—The differences across all possible haplotype frequencies were tested using the likelihood-ratio statistic calculated from the estimated haplotype-frequency likelihood [Zhao, J. H., D. Curtis, et al. (2000). Model-free analysis and permutation tests for allelic associations. Hum Hered 50(2): 133-9; Fallin, D., A. Cohen, et al. (2001). Genetic analysis of case/control data using estimated haplotype frequencies: application to APOE locus variation and Alzheimer's disease. Genome Res 11(1): 143-51]. The overall significance was tested across male and female 2×2 tables by means of the Cochran-Mantel-Haenszel test.

### Example 1

### SNPS in the COMT Locus are Asociated with Schizophrenia

Various studies have tested the possible association of the nonsynonymous polymorphism V158M of SEQ ID NO:29 (or V108M of SEQ ID NO:30) in the catechol-O-methyltransferase (COMT) gene with schizophrenia. However, these studies produced inconclusive results [Kunugi et al. (1997). Psychiatr. Genet. 7: 97-101; Li et al. (1996). Psychiatr. Genet. 6: 131-133; Li et al. (2000). Mol. Psychiatry 5: 77-84; Karayiorgou et al. (1998). Biol. Psychiatry 43: 425-431; Palmatier et al. (1997). PNAS: 94: 587-592]. To test the hypothesis that a functional polymorphism in the COMT gene increases the risk for schizophrenia, a comprehensive case-control study was conducted using the homogenous Ashkenazi Jewish population with SNPs from the COMT locus.

Experimental Results

Association of the SNPs in the COMT locus by population-based pool genotyping—PCR products were prepared from pooled DNA samples of about 300 schizophrenia cases (SZP) and 1000 control individuals (CTL), and allele frequency was determined using the Pyrosequencer™. The calculated difference in allele frequency between case and control was tested for significance using a standard χ² analysis. A striking difference of 8.5% in allele frequency was observed with SNP rs4633 (Table 3). SNPs rs4680, rs165599 and rs6269 exhibited about 6.1-6.3% difference in allele frequency and SNP rs737865 exhibited a more moderate, but still significant, difference of 4% in allele frequency between schizophrenia cases and control individuals (Table 1). Thus, these results suggest that SNPs in the COMT locus are associated with increased risk for schizophrenia and suggest their further analysis by a large-scale individual genotyping.

TABLE 3
Allele frequency differences of SNPs in the COMT locus
in a schizophrenia case-control study

| SEQ ID | | Allele frequency |
|---|---|---|
| NO: | NCBI SNP ID# | difference |
| 1 | rs737865 | 4.0% |
| 5 | rs4680 | 6.5% |
| 9 | rs165599 | 6.1% |
| 13 | rs6269 | 6.4% |
| 17 | rs4633 | 8.5% |

Differences in allele frequencies of SNPs in the COMT locus determined by pooled DNA genotyping of about 300 schizophrenia cases and 1000 control individuals.

Linkage disequilibrium between SNPs in the COMT locus—To determine the linkage disequilibrium (LD) status of SNPs in the COMT locus in the Ashkenazi Jewish population SNP genotyping was performed on DNA samples from 70-90 control individuals. SNPs rs6269 and rs4633 were in complete LD (D′=1 for both SNPs) with SNP rs4680. SNP rs362204 (rs3838146) was in complete LD with rs165599 (D′=1, r²=0.84). In addition, a high degree of LD was found between SNPs rs737865 and rs4680 (D′=0.85). On the other hand, SNPs rs165599 exhibited lower LD with SNP rs4680 and rs737865 (D′=0.58 and D′=0.32, respectively). Moreover, SNP rs2020917 and SNP 2 [Saito, S., A. Iida, et al. (2001). Identification of 197 genetic variations in six human methyltranferase genes in the Japanese population. J Hum Genet 46(9): 529-37, SEQ ID NO:28] were in complete LD with SNP rs737865 (D′=1, r²=0.98 and D′=1, r²=0.32, respectively). SNP 3 [Saito, S., A. Iida, et al. (2001). Identification of 197 genetic variations in six human methyltranferase genes in the Japanese population. J Hum Genet 46(9): 529-37, SEQ ID NO:27] is estimated to be in high LD with rs737865, since it is located between rs2020917 and SNP 2, which are in completed LD with each other and with SNP rs737865. Altogether, these results suggest that SNPs rs4680, rs165599 and rs737865 represent independent polymorphisms in the COMT locus.

Therefore, to substantiate the association between SNPs in the COMT locus and increased risk for schizophrenia, a large-scale individual genotyping was conducted using the rs4680, rs165599 and rs737865 SNPs.

The high activity form of the COMT enzyme is associated with increased risk to schizophrenia—Genotyping of 835 schizophrenia cases and 4081 control individuals was performed using the Pyrosequencing and Acycloprime technologies. As shown in Table 4 hereinbelow the G allele encoding the high activity form of the COMT enzyme (V158 of SEQ ID NO:29 or V108 of SEQ ID NO:30) was more frequent in schizophrenia males (56%) than in controls [52%, p=4.81×10⁻³ (Table 4), odds ratio (OR) 1.20; 95% C.I. 1.06-1.36, (Table 5)]. A similar and significant difference in allele frequencies was observed when all schizophrenia cases (males and females) were compared with controls (p=4.80×10⁻³, Table 4). In addition, the GG genotype was more frequent among schizophrenia males than in the controls [p=1.03×10⁻² (Table 4), OR 1.27; 95% C.I. 1.05-1.55 (Table 5)]. A modest difference was observed between schizophrenia females and control females (3%, p=0.113, Table 4). In addition, the predisposition probabilities to schizophrenia are 40% and 19% higher for the homozygous and the heterozygous genotypes, respectively, over the protective genotype (GRR, Table 5). Similarly, the predisposition probabilities to schizophrenia in males are 44% and 16% higher for the homozygous and the heterozygous genotypes, respectively (GRR, Table 5). Altogether, these results demonstrate that the G allele and the GG genotype of SNP rs4680 are associated with schizophrenia.

TABLE 4
Allele and genotype frequencies of SNP rs4680 in schizophrenia case-control
study.

| | Affected | Control | Allele | AA genotype | GG genotype |
|---|---|---|---|---|---|
| Studied Group | (G Freq.) | (G Freq.) | P value | P value | P value |
| SZP A/CTL | 835 (0.55) | 4081 (0.52) | 4.80 × 10⁻³ | 2.50 × 10⁻² | 1.61 × 10⁻² |
| SZP M/CTL M | 537 (0.56) | 3034 (0.52) | 1.12 × 10⁻² | 9.88 × 10⁻² | 1.16 × 10⁻² |
| SZP F/CTL F | 291 (0.54) | 1041 (0.51) | 1.13 × 10⁻¹ | 4.55 × 10⁻² | 5.38 × 10⁻¹ |
| SZP M/CTL | 537 (0.56) | 4081 (0.52) | 4.81 × 10⁻³ | 3.83 × 10⁻² | 1.03 × 10⁻² |

Allele and genotype frequencies revealed by individual genotyping.
Affected = schizophrenia (SZP) cases;
Control (CTL) = healthy individuals;
P values reflect the significance of differences between cases and controls;
M = males,
F = females,
A = all (males and females).

TABLE 5
Significant genotype relative risk (GRR) and odds ratio (OR) of SNPs rs4680, rs165599
and rs737865 associated with schizophrenia

| | | Allele OR (95% | GRR | GRR | Risk genotype, |
|---|---|---|---|---|---|
| SNP ID# | Studied Group | C.I.) | (risk genotype) | (heterozygote) | OR (95% C.I.) |
| rs4680 | SZP A/CTL | 1.16 (1.04-1.29) | 1.40 (GG) | 1.19 | 1.22 (1.04-1.44) |
| rs4680 | SZP M/CTL | 1.20 (1.06-1.36) | 1.44 (GG) | 1.16 | 1.27 (1.05-1.55) |
| rs165599 | SZP A/CTL | 1.23 (1.11-1.37) | 1.56 (GG) | 1.15 | 1.44 (1.19-1.74) |
| rs165599 | SZP F/CTL F | 1.49 (1.24-1.80) | 2.23 (GG) | 1.27 | 1.96 (1.40-2.74) |
| rs737865 | SZP A/CTL | 1.22 (1.09-1.36) | 1.53 (CC) | 1.17 | 1.39 (1.15-1.69) |
| rs737865 | SZP M/CTL M | 1.18 (1.03-1.35) | 1.46 (CC) | NS | 1.48 (1.17-1.86) |
| rs737865 | SZP F/CTL F | 1.36 (1.12-1.65) | 1.83 (CC) | 1.70 | NS |

Genotype relative risk (GRR) and odds ratio (OR) revealed by individual genotyping.
SZP = schizophrenia cases;
CTL = healthy controls;
C.I. = confidence interval;
A = all (males and females),
M = males,
F = females;
NS = non significant.

To substantiate the association between the COMT gene and increased risk for schizophrenia, additional SNPs in the COMT locus were analyzed.

The G allele and the GG genotype of SNP rs165599 are risk factors for schizophrenia in females—Genotyping of 294 schizophrenia female cases and 1057 control females by the Pyrosequencing technology and restriction analysis revealed that the G allele of SNP rs165599 was much more frequent among schizophrenia females (44%) than among control females [35%, p=1.90×10⁻⁵ (Table 6), OR 1.49; 95% C.I. 1.24-1.80 (Table 5)]. In addition, the GG genotype was strongly associated with schizophrenia in females [p=2.58×10⁻⁵ (Table 6), OR 1.96; 95% C.I. 1.40-2.74 (Table 5)]. Moreover, comparison of all schizophrenia cases (males and females) with controls revealed a strong association of the G allele and the GG genotype with schizophrenia (G allele, p=8.14×10⁻⁵; GG genotype, p=1.29×10⁻⁴, Table 6). Thus, these results suggest that the G allele and/or the GG genotype of SNP rs165599 increase the risk for schizophrenia in the general population and in females in particular. Indeed, the predisposition probabilities to schizophrenia are 56% and 15% higher for the homozygous (GG) and the heterozygous (GA) genotypes in all schizophrenia cases, respectively (GRR, Table 5). In female, the predisposition probabilities to schizophrenia were found to be 123% higher for the homozygous risk genotype and 27% higher for the heterozygous genotype over the protective genotype (GRR, Table 5). To further evaluate the effect of SNP rs165599 on schizophrenia, the population attributable risk (PAR) was calculated for the protective genotype (AA) and demonstrated that 21% of the schizophrenic females would not have been affected if the population was monomorphic for the AA genotype (Table 7).

TABLE 6
Allele and genotype frequencies of SNP rs165599 in schizophrenia case-control
study.

| | Affected | Control | Allele | AA genotype | GG genotype |
|---|---|---|---|---|---|
| Studied Group | (G Freq.) | (G Freq.) | P value | P value | P value |
| SZP A/CTL | 848 (0.43) | 4161 (0.38) | 8.14 × 10⁻⁵ | 5.19 × 10⁻³ | 1.29 × 10⁻⁴ |
| SZP M/CTL M | 547 (0.42) | 3098 (0.38) | 2.93 × 10⁻² | 1.02 × 10⁻¹ | 4.56 × 10⁻² |
| SZP F/CTL F | 294 (0.44) | 1057 (0.35) | 1.90 × 10⁻⁵ | 2.87 × 10⁻³ | 2.58 × 10⁻⁵ |

Allele and genotype frequencies revealed by individual genotyping.
Affected = schizophrenia (SZP) cases;
Control (CTL) = healthy individuals;
P values reflect the significance of differences between cases and controls.
M = males,
F = females,
A = all males and females.

TABLE 7
Most Significant population attributable risk
(PAR) in schizophrenia

| SNP ID# | Studied Group | PAR |
|---|---|---|
| rs4680 | SZP F/CTL F | 24% |
| rs165599 | SZP F/CTL F | 21% |
| rs737865 | SZP F/CTL F | 31% |

Population attributable risk (PAR) revealed by individual genotyping.
SZP = schizophrenia cases;
CTL = healthy controls,
F = females.

The C allele and the CC genotype of SNP rs737865 are risk factors for schizophrenia—To test the association between SNP rs737865 and schizophrenia, 844 schizophrenia cases and 2951 control individuals were subjected to SNP genotyping using restriction analysis. As is shown in Table 8, the T allele was significantly more prevalent among control individuals than among schizophrenia cases (p=3.95×10⁻⁴). In addition, the T allele and the TT genotype were significantly more prevalent among healthy females than among schizophrenic females (p=2.27×10⁻³ and p=8.79×10⁻⁴, respectively, Table 8), suggesting that the T allele and the TT genotype of SNP rs737865 are protective factors against schizophrenia in females. Indeed, the calculated PAR for females was 31% in females (Table 7). Further analysis revealed high association of the CC genotype with schizophrenia in the general population [p=5.54×10⁻⁴ (Table 8), OR 1.39; 95% C.I. 1.15-1.69 (Table 5)] and in males [p=1.02×10⁻³ (Table 8), risk genotype GRR=1.46, OR 1.48; 95% C.I. 1.17-1.86 (Table 5)]. In addition, the predisposition probabilities to schizophrenia for the homozygous and heterozygous genotypes are 53% and 17%, respectively, higher than of the protective TT genotype (GRR, Table 5). Moreover, the predisposition probabilities of the homozygous CC and the heterozygote CT genotypes in females are 83% and 70%, respectively, higher than of females with the protective TT genotype (GRR, Table 5). Altogether, these results demonstrate that the C allele and/or the CC genotype of SNP rs737865 are risk factors for schizophrenia in the general population and that the T allele and the TT genotype are protective factors against schizophrenia in females.

TABLE 8
Allele and genotype frequencies of SNP rs737865 in schizophrenia case-control
study.

| | Affected | Control | Allele | CC genotype | ZTT genotype |
|---|---|---|---|---|---|
| Studied Group | (T Freq.) | (T Freq.) | P value | P value | P value |
| SZP A/CTL | 844 (0.55) | 2951 (0.60) | 3.95 × 10⁻⁴ | 5.54 × 10⁻⁴ | 1.27 × 10⁻² |
| SZP M/CTL M | 546 (0.55) | 2231 (0.59) | 1.47 × 10⁻² | 1.02 × 10⁻³ | 3.17 × 10⁻¹ |
| SZP F/CTL F | 291 (0.54) | 716 (0.62) | 2.27 × 10⁻³ | 1.52 × 10⁻¹ | 8.79 × 10⁻⁴ |

Allele and genotype frequencies revealed by individual genotyping.
Affected = schizophrenia (SZP) cases;
Control (CTL) = healthy individuals;
P values reflect the significance of differences between cases and controls.
M = males,
F = females,
A = all (males and females).

Increased risk to schizophrenia due to the C-G-G haplotype in the COMT locus—To substantiate the association between schizophrenia and SNPs in the COMT locus the frequencies of all possible two-SNP and three-SNP haplotypes were compared between schizophrenia cases and controls. A strong association with schizophrenia was observed with the three-SNP haplotype (p=1.28×10⁻⁵, Table 9). Of them, the C-G-G haplotype of SNPs rs737865, rs4680 and rs165599 exhibited the most significant association with schizophrenia (p=1.06×10⁻⁷, Table 9). In particular, a strong association of the C-G-G haplotype was observed in females [p=8.87×10⁻⁵, (Table 9), OR 1.73; 95% C.I. 1.38-2.17 (Table 10)). These results suggest that the C-G-G haplotype of SNPs rs737865-rs4680-rs165599 is a risk factor for all schizophrenia cases. Indeed, the predisposition probability of the CC-GG-GG triple haplotype in the general population is 105% higher over the protective triple genotype (GRR, Table 10). In addition, the predisposition probability of the triple risk genotype was 82% and 212% higher over the protective triple genotype for females and males, respectively (GRR, Table 10).

To further evaluate the effect of SNPs rs737865 and rs165599 on schizophrenia, the PAR value of the double genotype TT-AA was calculated. This analysis revealed that 9% of schizophrenic males, 31% of schizophrenic females and 15% of all schizophrenia cases (males and females) would not have been affected if the population was monomorphic for the TT-AA double genotype (Table 11).

TABLE 9
Most significant haplotypes of SNPs rs737865, rs4680 and rs165599 in schizophrenia.

| | | | | Combined | |
|---|---|---|---|---|---|
| | | Number of | Number | Haplotype | Best Haplotype |
| SNP ID# | Studied Group | affected | of control | P-value | P value |
| rs737865/rs4680/ | SZP/CTL | 831 | 2917 | 1.28 × 10⁻⁵ | 1.06 × 10⁻⁷ (CGG) |
| rs165599 | | | | | |
| rs737865/rs4680/ | SZP F/CTL F | 288 | 707 | 4.22 × 10⁻⁵ | 2.06 × 10⁻⁶ (CGG) |
| rs165599 | | | | | |
| rs737865/rs4680/ | SZP M/CTL M | 538 | 2206 | 1.05 × 10⁻² | 4.54 × 10⁻⁴ (CGG) |
| rs165599 | | | | | |

Haplotype analysis revealed by individual genotyping. P values represent the significance of differences between cases and controls.
SZP = schizophrenia cases;
CTL = healthy controls,
M = males,
F = females.

TABLE 10
Genotype relative risk (GRR) and odds ratio (OR) of the C-G-G
haplotype of SNPs rs737865-rs4680-rs165599 in schizophrenia.

| Studied Group | GRR | OR (95% C.I.) |
|---|---|---|
| SZP/CTL | 2.05 | 1.40 (1.24-1.59) |
| SZP F/CTL F | 1.82 | 1.73 (1.38-2.17) |
| SZP M/CTL M | 3.12 | 1.32 (1.13-1.53) |

Haplotype genotype relative risk (GRR) of the risk triple homozygote (CC-GG-GG) and odds ratio (OR) revealed by individual genotyping.
C.I. = confidence interval,
SZP = schizophrenia cases;
CTL = healthy controls,
M = males,
F = females.

TABLE 11
Population attributable risk (PAR) for the TT-AA
double genotype of SNPs rs737865 and rs165599

| Studied Group | PAR |
|---|---|
| SZP/CTL | 15% |
| SZP M/CTL M | 9% |
| SZP F/CTL F | 31% |

Population attributable risk (PAR) revealed by individual genotyping.
SZP = schizophrenia cases;
CTL = healthy controls,
M = males,
F = females.

Altogether these results demonstrate a high association of SNPs in the COMT locus with schizophrenia. Moreover, haplotype analysis revealed that the C-G-G haplotype of SNPs rs737865-rs4680-rs165599 is a risk factor for schizophrenia. These results suggest the use of SNPs rs737865, rs4680 and rs165599 and any of the SNPs in LD with them for the diagnosis of schizophrenia cases and for the prediction of an individual's risk of suffering from schizophrenia.

### Example 2

### Genotype-Dependent Drug Efficacy Using the COMT SNPS

The common treatment regimes of schizophrenia are based on reducing the severity of the symptoms and include the use of antipsychotics and neuroleptics drugs. However, these drugs have only limited effect on many patients while causing severe side effects in others. To understand the reasons for high variability in drug responsiveness a comprehensive genotype-phenotype analysis was performed using patients' questionnaire and their genotypic status for SNPs in the COMT locus.

Experimental Results

Genotype subclasses in the COMT locus display different responsiveness towards schizophrenia treatment—The genotypic status of SNPs rs4680 and rs165599 in the COMT locus were tested for association with patient's drug responsiveness. This analysis revealed that the GG genotype of SNP rs4680 was correlated with high efficiencies of thioridazine treatment in females (p=1.25×10⁻³) and of clozapine treatment in males (p=4.04×10⁻³). In addition, the AA genotype of SNP rs165599 was correlated with high efficiency of thioridazine treatment in males (p=8.3×10⁻⁴). Furthermore, adverse effects to haloperidol treatment were correlated with the AG genotype of SNP rs165599 in females (p=9.26×10⁻⁴).

Altogether, these results demonstrate that variability in drug responsiveness could result from specific genotypic status of SNPs in the COMT locus. Furthermore, these results suggest the use of SNPs in the COMT locus for the prediction of drug responsiveness and effectiveness of schizophrenia drugs.

### Example 3

### SNPS in the COMT Locus are Asociated with Bipolar Disorder

To test the association between SNPs in the COMT gene and bipolar disorder, a comprehensive case-control study was conducted on the homogenous Ashkenazi Jewish population and with three independent SNPs in the COMT locus.

Experimental Results

SNPs in the COMT locus are associated with bipolar disorders (BIP)-Genotyping of 217 bipolar disorder cases and 4091 controls was performed using SNPs rs4680, rs165599 and rs737865. The G allele of SNP rs165599 was significantly associated with bipolar females [p=5.1×10⁻³ (Table 12), OR 1.25; 95% C.I. 0.95-1.64 (Table 14)]. A modest association was found with the C allele of SNP rs737865 (p=0.032, Table 13). In addition, the GG and CC genotypes of SNPs rs165599 and rs737865, respectively, were significantly associated with bipolar females (OR 1.48; 95% C.I. 1.12-1.96 and OR 1.37; 95% C.I. 1.03-1.82, respectively, Table 14). Moreover, the predisposition probability of the homozygous GG and the heterozygous AG genotypes of SNP rs165599 in females are 104% and 93% higher than that of the protective AA genotype, respectively.

TABLE 12
Allele frequencies of SNPs rs4680 and rs165599 in bipolar
(BIP) disorders case-control study

| | Studied | Affected | Control | Allele |
|---|---|---|---|---|
| SNP ID | Group | (G Freq.) | (G Freq.) | P value |
| rs4680 | BIP M/CTL M | 104 (0.538) | 3034 (0.52) | NS |
| rs4680 | BIP F/CTL F | 110 (0.564) | 1041 (0.51) | NS |
| rs165599 | BIP M/CTL M | 107 (0.444) | 3034 (0.38) | NS |
| rs165599 | BIP F/CTL F | 110 (0.441) | 1057 (0.35) | 5.1 × 10⁻³ |

Allele frequencies revealed by individual genotyping.
Affected = bipolar disorder (BIP) cases;
Control (CTL) = healthy individuals;
P values reflect the significance of differences between cases and controls.
M = males,
F = females.
NS = non significant.

TABLE 13
Allele frequencies of SNP rs737865 in bipolar (BIP)
disorder case-control study

| | Affected | Control | Allele |
|---|---|---|---|
| Studied Group | (C Freq.) | (C Freq.) | P value |
| BIP M/CTL M | 105 (0.424) | 2231 (0.41) | NS |
| BIP F/CTL F | 109 (0.463) | 716 (0.38) | 0.032 |

Allele frequencies revealed by individual genotyping.
Affected = bipolar disorder (BIP) cases;
Control (CTL) = healthy individuals;
P values reflect the significance of differences between cases and controls.
M = males,
F = females.
NS = non significant.

TABLE 14
Most significant allele odds ratio (OR) of SNPs rs4680,
rs165599 and rs737865 associated with bipolar disorder

| | Allele OR |
|---|---|
| SNP | (95% C.I.) in females |
| rs4680 | NS |
| rs165599 | 1.48 (1.12-1.96) |
| rs737865 | 1.37 (1.03-1.82) |

Allele odds ratio (OR) revealed by individual genotyping.
C.I. = confidence interval,
NS = non-significant.

Increased risk for bipolar disorder due to specific haplotypes in the COMT locus—To substantiate the association between bipolar disorder and SNPs in the COMT locus, the frequencies of all possible haplotypes were compared between bipolar cases and controls. A strong association of bipolar disorder in females was observed with the C-G haplotype of SNPs rs737865-165599 [p=1.8×10⁻³, OR 1.67; 95% C.I. 1.21-2.30 (Table 15)] and a modest association was observed with the C-G-G haplotype of SNPs rs737865-165599-4680 [p=0.024, OR 1.47; 95% C.I. 1.05-2.06 (Table 15)]. In addition, the increased predisposition risk of the CC-GG double homozygote is 65% higher than that of the protective double homozygote (i.e., TT-AA, GRR, Table 15). These results suggest that the C-G haplotype of SNPs rs737865-rs165599 is a risk factor for bipolar disorder in females.

TABLE 15
Significant haplotype genotype relative risk (GRR) and odds ratio
(OR) of SNPs rs4680, rs165599 and rs737865 associated with bipolar
disorder

| | | Haplotype OR | |
|---|---|---|---|
| Haplotype | GRR | (95% C.I.) in females | P value |
| C-G (rs737865-165599) | 1.65 | 1.67 (1.21-2.30) | 0.0018 |
| C-G-G (rs737865-165599-4680) | ND | 1.47 (1.05-2.06) | 0.024 |

Haplotype odds ratio (OR) revealed by individual genotyping.
P values represent the significance of differences between cases and controls;
C.I. = confidence interval;
ND = not determined.

Altogether, these results demonstrate that SNPs rs737865 and rs165599 are strongly associated with bipolar disorder in females and, more particularly, that the C-G haplotype of SNPs rs737865-165599 significantly increases the risk of bipolar disorder. Furthermore, these results suggest the use of SNPs rs737865, rs165599 and rs4680 and any SNPs in LD with them for the diagnosis of bipolar disorder in females and for the prediction of an individual's risk of suffering from bipolar disorder.

### Example 4

### SNPS in the COMT Locus are Associated with Breast Cancer

Several studies have reported inconclusive results on the association between the low activity form of the COMT enzyme and breast cancer [Thompson, P. A., P. G. Shields, et al. (1998). Genetic polymorphisms in catechol-O-methyltransferase, menopausal status, and breast cancer risk. Cancer Res 58(10): 2107-10; Lavigne, J. A., K. J. Helzlsouer, et al. (1997). An association between the allele coding for a low activity variant of catechol-O-methyltransferase and the risk for breast cancer. Cancer Res 57(24): 5493-7; Matsui, A., T. Ikeda, et al. (2000). Progression of human breast cancers to the metastatic state is linked to genotypes of catechol-O-methyltransferase. Cancer Lett 150(1): 23-31; Yim, D. S., S. K. Parkb, et al. (2001). Relationship between the Val158Met polymorphism of catechol O-methyl transferase and breast cancer. Pharmacogenetics 11(4): 279-86; Millikan, R. C., G. S. Pittman, et al. (1998). Catechol-O-methyltransferase and breast cancer risk. Carcinogenesis 19(11): 1943-7; Bergman-Jungestrom, M. and S. Wingren (2001). Catechol-O-Methyltransferase (COMT) gene polymorphism and breast cancer risk in young women. Br J Cancer 85(6): 859-62; Hamajima, N., K. Matsuo, et al. (2001). Limited association between a catechol-O-methyltransferase (COMT) polymorphism and breast cancer risk in Japan. Int J Clin Oncol 6(1): 13-8; Kocabas, N. A., S. Sardas, et al. (2002). Cytochrome P450 CYP1B1 and catechol O-methyltransferase (COMT) genetic polymorphisms and breast cancer susceptibility in a Turkish population. Arch Toxicol. 76(11): 643-9]. To test the association between the COMT enzyme and breast cancer, a comprehensive breast cancer case-control study was conducted on the Ashkenazi Jewish population using SNPs from the COMT locus.

Experimental Results

The COMT high activity allele is a risk factor for breast cancer—To test the association between the COMT gene and breast cancer, DNA samples of 834 cases and 4081 control individuals were genotyped. In sharp contrast to previous publications [Thompson, P. A., P. G. Shields, et al. (1998). Genetic polymorphisms in catechol-O-methyltransferase, menopausal status, and breast cancer risk. Cancer Res 58(10): 2107-10; Lavigne, J. A., K. J. Helzlsouer, et al. (1997). An association between the allele coding for a low activity variant of catechol-O-methyltransferase and the risk for breast cancer. Cancer Res 57(24): 5493-7; Matsui, A., T. Ikeda, et al. (2000). Progression of human breast cancers to the metastatic state is linked to genotypes of catechol-O-methyltransferase. Cancer Lett 150(1): 23-31; Yim, D. S., S. K. Parkb, et al. (2001). Relationship between the Val158Met polymorphism of catechol O-methyl transferase and breast cancer. Pharmacogenetics 11(4): 279-86] the high activity form of the COMT enzyme (V158 of SEQ ID NO:29 or V108 of SEQ ID NO:30) encoded by the G allele of SNP rs4680 was strongly associated with breast cancer [p=8.18×10⁻³ (Table 16), OR 1.15; 95% C.I. 1.04-1.28 (Table 17)]. In addition, the homozygous GG genotype was highly associated to BRC [p=3.76×10⁻⁴ (Table 16), OR 1.34; 95% C.I. 1.14-1.58 (Table 17)]. These results suggest that the GG genotype of SNP rs4680 is a risk genotype for breast cancer. Indeed, the predisposition probability of the GG genotype is 35% higher than the protective genotype (GRR, Table 17). Moreover, stratification of the case population by age of onset (AOO) lower or higher than 50 years of age, familial or sporadic disease and aggressiveness of the disease (i.e., malignancy existence in, or absence from lymph nodes) revealed a significant association of the high activity COMT with late-onset disease [G allele, p=4.61×10⁻³; GG genotype, p=5.07×10⁻⁴, (Table 16), GRR=1.28 (Table 17)], with non-familial breast cancer (GG genotype, p=2.46×10⁻³ (Table 16), GRR=1.32 (Table 17)], and with increased risk of developing a more aggressive form of breast cancer (GG genotype, p=3.07×10⁻³ (Table 16), GRR=1.50 (Table 17)]. The overall calculated effect of this SNP on breast cancer (PAR value) was 8.7%, suggesting that 8.7% of cases would not have been affected if the population was monomorphic for the A allele of SNP rs4680. Altogether, these results demonstrate a strong association of the high activity COMT allele with breast cancer, and more particularly, association of the GG genotype of SNP rs4680 with a late-onset, non-familial and aggressive form of breast cancer.

TABLE 16
Allele and genotype frequencies of SNP rs4680 in
breast cancer case-control study.

| | Affected | Control | Allele | GG genotype |
|---|---|---|---|---|
| Studied Group | (G Freq.) | (G Freq.) | P value | P value |
| BRC/CTL | 834 (0.552) | 4081 (0.517) | 8.18 × 10⁻³ | 3.76 × 10⁻⁴ |
| BRC; | 215 (0.537) | 4081 (0.517) | 4.06 × 10⁻¹ | 1.19 × 10⁻¹ |
| AOO <50/CTL | | | | |
| BRC; | 594 (0.561) | 4081 (0.517) | 4.61 × 10⁻³ | 5.07 × 10⁻⁴ |
| AOO >50/CTL | | | | |
| BRC; Not | 331 (0.563) | 4081 (0.517) | 2.05 × 10⁻² | 2.46 × 10⁻³ |
| Familial/CTL | | | | |
| BRC; | 491 (0.543) | 4081 (0.517) | 1.21 × 10⁻¹ | 2.12 × 10⁻¹ |
| Familial/CTL | | | | |
| BRC; | 214 (0.579) | 4081 (0.517) | 1.12 × 10⁻² | 3.07 × 10⁻³ |
| Nodes (+)/CTL | | | | |
| BRC; | 415 (0.525) | 4081 (0.517) | 6.35 × 10⁻¹ | 2.63 × 10⁻¹ |
| Nodes (−)/CTL | | | | |

Allele and genotype frequencies revealed by individual genotyping.
Affected = breast cancer (BRC) cases;
Control (CTL) = healthy individuals;
P values reflect the significance of differences between cases and controls.
AOO = age of onset,
Nodes (+/−) = presence or absence of malignancy in lymph nodes.

TABLE 17
Significant genotype relative risk (GRR) and odds ratio (OR) of SNPs
rs4680 and rs165599 associated with breast cancer

| | | Allele OR | GRR | Risk genotype OR |
|---|---|---|---|---|
| SNP ID# | Studied Group | (95% C.I.) | (risk genotype) | (95% C.I.) |
| rs4680 | BRC/CTL | 1.15 (1.04-1.28) | 1.35 (GG) | 1.34 (1.14-1.58) |
| rs4680 | BRC; AOO > 50/ | 1.17 (1.04-1.32) | 1.28 (GG) | 1.32 (1.11-1.59) |
| | CTL | | | |
| rs4680 | BRC; Not | 1.18 (1.01-1.38) | 1.32 (GG) | 1.40 (1.11-1.77) |
| | Familial/CTL | | | |
| rs4680 | BRC; Nodes | 1.27 (1.04-1.54) | 1.50 (GG) | 1.46 (1.09-1.94) |
| | (+)/CTL | | | |
| rs165599 | BRC F/CTL F | 1.34 (1.17-1.52) | 1.85 (GG) | 1.60 (1.24-2.07) |
| rs165599 | BRC; AOO > 50/ | 1.19 (1.06-1.35) | 1.51 (GG) | 1.43 (1.15-1.78) |
| | CTL | | | |
| rs165599 | BRC; Not | 1.21 (1.03-1.42) | 1.44 (GG) | NS |
| | Familial/CTL | | | |
| rs165599 | BRC; Nodes | 1.28 (1.06-1.56) | 1.17 (GG) | 1.44 (1.01-2.04) |
| | (+)/CTL | | | |

Genotype relative risk (GRR) and odds ratio (OR) revealed by individual genotyping.
BRC = breast cancer cases;
CTL = healthy controls,
C.I. = confidence interval,
F = females,
NS = not significant.

The T-G haplotype of SNPs rs737865-4680 is a risk factor for breast cancer—Although genotyping of SNP rs737865 resulted only in a modest association with breast cancer (p=2.57×10⁻², Table 18) haplotype analysis of SNPs rs737865 and rs4680 revealed high association with breast cancer (p=4.17×10⁻⁵). The strongest association with breast cancer was observed with the T-G haplotype of SNPs rs737865-rs4680 (p=1.69×10⁻⁴, OR 1.33; 95% C.I. 1.15-1.53). The predisposition risk of the TT-GG double homozygote is 155% higher over the protective double homozygote (CC-AA).

TABLE 18
Allele and genotype frequencies of SNP rs737865 in
breast cancer case-control study.

| Studied | Affected | Control | Allele | TT genotype |
|---|---|---|---|---|
| Group | (T Freq.) | (T Freq.) | P value | P value |
| BRC/CTL | 833 (0.616) | 2951 (0.597) | 1.60 × 10⁻¹ | 2.57 × 10⁻² |
| BRC; AOO < | 217 (0.615) | 2951 (0.597) | 4.49 × 10⁻¹ | 2.61 × 10⁻¹ |
| 50/CTL | | | | |
| BRC; AOO > | 590 (0.612) | 2951 (0.597) | 3.33 × 10⁻¹ | 7.26 × 10⁻² |
| 50/CTL | | | | |
| BRC; Not | 330 (0.609) | 2951 (0.597) | 5.40 × 10⁻¹ | 2.42 × 10⁻¹ |
| Familial/CTL | | | | |
| BRC; | 491 (0.625) | 2951 (0.597) | 9.12 × 10⁻² | 2.64 × 10⁻² |
| Familial/CTL | | | | |
| BRC; Nodes | 216 (0.609) | 2951 (0.597) | 6.22 × 10⁻¹ | 1.90 × 10⁻¹ |
| (+)/CTL | | | | |
| BRC; Nodes | 414 (0.634) | 2951 (0.597) | 4.00 × 10⁻² | 5.03 × 10⁻² |
| (−)/CTL | | | | |

Allele and genotype frequencies revealed by individual genotyping.
Affected = breast cancer (BRC) cases;
Control (CTL) = healthy individuals;
P values reflect the significance of differences between cases and controls.
AOO = age of onset,
Nodes (+/−) = presence or absence of malignancy in lymph nodes.

High association of SNP rs165599 with increased risk to breast cancer—To further substantiate the association of the COMT gene with breast cancer genotyping was performed with the rs165599 SNP. The G allele was more prevalent in breast cancer cases (41.4%) than in control individuals (37.5%, p=2.23×10⁻³, Table 19). Accordingly, the GG genotype was highly associated with breast cancer [(p=4.17×10⁻³, (Table 19), OR 1.60; 95% C.I. 1.24-2.07 (Table 17)] suggesting that it is a breast cancer risk factor. Indeed, the predisposition probability of the GG genotype is 85% higher than the protective genotype (GRR, Table 17). When the case population was stratified by AOO lower or higher than 50 years of age, higher association to the GG genotype was observed in patients with late-onset disease [p=2.72×10⁻⁴ (Table 19), GRR=1.51 (Table 17)] than in patients with early-onset disease (p=8.65×10⁻¹, Table 19). In addition, the G allele and the GG genotype were significantly associated with a non-familial breast cancer [allele p value=8.42×10⁻³ (Table 19), risk genotype GRR=1.44 (Table 17)] and with a relatively more aggressive form of breast cancer having lymph nodes metastases [allele p value=8.63×10⁻³ (Table 19), risk genotype GRR=1.17, OR 1.44; 95% C.I. 1.01-2.04 (Table 17)]. The overall calculated effect of this SNP on breast cancer (PAR value) was 19%, demonstrating a significant contribution of this SNP towards breast cancer.

TABLE 19
Allele and genotype frequencies of SNP rs165599 in
breast cancer case-control study

| Studied | Affected | Control | Allele | GG genotype |
|---|---|---|---|---|
| Group | (G Freq.) | (G Freq.) | P value | P value |
| BRC/CTL | 836 (0.414) | 4161 (0.375) | 2.23 × 10⁻³ | 4.17 × 10⁻³ |
| BRC; AOO < | 217 (0.410) | 4161 (0.375) | 1.37 × 10⁻¹ | 8.65 × 10⁻¹ |
| 50/CTL | | | | |
| BRC; AOO > | 593 (0.422) | 4161 (0.375) | 1.53 × 10⁻³ | 2.72 × 10⁻⁴ |
| 50/CTL | | | | |
| BRC; Not | 332 (0.426) | 4161 (0.375) | 8.42 × 10⁻³ | 6.19 × 10⁻² |
| Familial/CTL | | | | |
| BRC; | 492 (0.412) | 4161 (0.375) | 2.40 × 10⁻² | 1.34 × 10⁻² |
| Familial/CTL | | | | |
| BRC; Nodes | 216 (0.438) | 4161 (0.375) | 8.63 × 10⁻³ | 5.00 × 10⁻² |
| (+)/CTL | | | | |
| BRC; Nodes | 415 (0.399) | 4161 (0.375) | 1.71 × 10⁻¹ | 1.10 × 10⁻¹ |
| (−)/CTL | | | | |

Allele and genotype frequencies revealed by individual genotyping.
Affected = breast cancer (BRC) cases;
Control (CTL) = healthy individuals;
P values reflect the significance of differences between cases and controls.
AOO = age of onset,
Nodes (+/−) = presence or absence of metastasis in lymph nodes.

Altogether, these results demonstrate strong associations of the high activity form of the COMT enzyme encoded by the G allele of SNP rs4680 and of the G allele of SNP rs165599 with breast cancer. These results further suggest the use of SNPs rs4680 and rs165599 and any SNP in LD with them and/or the T-G haplotype of SNPs rs73785-rs

4680 in the diagnosis and prediction of prognosis of breast cancer.

### Example 5

### SNPS in the COMT Locus are Associated with Colorectal Cancer (CRC)

To define risk factors for colorectal cancer a comprehensive case-control study was conducted in the Ashkenazi Jewish population using SNPs from the COMT locus.

Experimental Results

The high activity form of the COMT enzyme is highly associated with increased risk for colorectal cancer—Genotyping of 938 CRC cases and 4018 controls revealed high association of the G allele and the GG genotype with CRC [allele, p=5.10×10⁻³ (Table 20); genotype, p=2.91×10⁻³ (Table 20); risk genotype GRR=1.37, OR 1.28; 95% C.I. 1.10-1.49 (Table 21)]. Further analysis based on the case's gender revealed that the difference in frequency of the G allele was much more prominent in female cases (allele frequency difference=4.4%, p=1.36×10⁻², Table 20) than in males (allele frequency difference=2.9%, p=8.32×10⁻², Table 20). Moreover, the calculated PAR in females was 21% (Table 22), suggesting a significant effect of this SNP on CRC females. Thus, these results demonstrate that the G allele and the GG genotype of SNP rs4680 are risk factors for CRC in general and in females in particular.

TABLE 20
Allele and genotype frequencies of SNP rs4680 in colorectal cancer case-control
study.

| Studied | Affected | Control | Allele | AA genotype | GG genotype |
|---|---|---|---|---|---|
| Group | (G Freq.) | (G Freq.) | P value | P value | P value |
| CRC/CTL | 932 (0.553) | 4018 (0.517) | 5.10 × 10⁻³ | 1.12 × 10⁻¹ | 2.91 × 10⁻³ |
| CRC M/CTL | 490 (0.546) | 4018 (0.517) | 8.32 × 10⁻² | 7.14 × 10⁻¹ | 1.52 × 10⁻² |
| CRC F/CTL | 438 (0.561) | 4018 (0.517) | 1.36 × 10⁻² | 3.93 × 10⁻² | 4.38 × 10⁻² |

Allele and genotype frequencies revealed by individual genotyping.
Affected = colorectal cancer (CRC) cases;
Control (CTL) = healthy individuals;
P values reflect the significance of differences between cases and controls.
M = males,
F = females.

TABLE 21
Significant genotype relative risk (GRR) and odds ratio (OR) of SNPs rs4680, rs165599 and
rs737865 associated with colorectal cancer.

| | | | GRR | | Risk genotype, |
|---|---|---|---|---|---|
| | | Allele OR | (risk | GRR | OR |
| SNP ID# | Studied Group | (95% C.I.) | homozygote) | (heterozygote) | (95% C.I.) |
| rs4680 | CRC/CTL | 1.16 (1.04-1.28) | 1.37 (GG) | 1.15 | 1.28 (1.10-1.49) |
| rs165599 | CRC/CTL | 1.18 (1.07-1.31) | 1.39 (GG) | 1.14 | 1.25 (1.03-1.52) |
| rs165599 | CRC F/CTL F | 1.34 (1.14-1.57) | 1.80 (GG) | 1.20 | 1.60 (1.18-2.17) |
| rs737865 | CRC/CTL | 1.18 (1.06-1.31) | 1.39 (CC) | 1.17 | 1.23 (1.02-1.49) |

Allele and genotype odds ratio (OR) revealed by individual genotyping.
CRC = colorectal cancer cases;
CTL = healthy controls,
C.I. = confidence interval,
F = females.

TABLE 22
Most significant population attributable risk (PAR) of
SNPs rs4680, rs165599 and rs737865 in colorectal cancer.

| SNP ID# | Studied Group | PAR |
|---|---|---|
| rs4680 | CRC F/CTL | 21% |
| rs165599 | CRC F/CTL F | 17% |
| rs737865 | CRC F/CTL | 17% |

Population attributable risk (PAR) revealed by individual genotyping.
CRC = colorectal cancer cases;
CTL = healthy controls,
F = females.

SNP rs165599 is highly associated with increased risk for colorectal cancer—To substantiate the association of CRC with the COMT locus 938 CRC cases and 4161 controls were further individually genotyped for SNP rs165599. A strong association was observed between the G allele (p=9.33×10⁻⁴, Table 23) and the GG genotype [p=3.48×10⁻³ (Table 23); risk genotype GRR=1.39, OR 1.25; 95% C.I. 1.03-1.52 (Table 21)] of SNP rs165599 with CRC. Stratification of the case and control populations by gender revealed a strong effect of this SNP in females [allele, p=3.21×10⁻⁴ (Table 23); genotype, p=1.9×10⁻³ (Table 23); risk genotype GRR=1.80, OR 1.60; 95% C.I. 1.18-2.17 (Table 21)] demonstrating that the G allele and the GG genotype of SNP rs165599 are risk factor for CRC in the population in general, and in females in particular. The overall effect of this SNP on CRC females was 17% (Table 22).

TABLE 23
Allele and genotype frequencies of SNP rs165599 in colorectal cancer case-control
study.

| | Affected | Control | Allele | AA genotype | GG genotype |
|---|---|---|---|---|---|
| Studied Group | (G Freq.) | (G Freq.) | P value | P value | P value |
| CRC/CTL | 938 (0.416) | 4161 (0.375) | 9.33 × 10⁻⁴ | 1.02 × 10⁻² | 3.48 × 10⁻³ |
| CRC M/CTL M | 493 (0.416) | 3098 (0.384) | 5.53 × 10⁻² | 1.05 × 10⁻¹ | 1.29 × 10⁻¹ |
| CRC F/CTL F | 441 (0.417) | 1057 (0.348) | 3.21 × 10⁻⁴ | 6.25 × 10⁻³ | 1.19 × 10⁻³ |

Allele and genotype frequencies revealed by individual genotyping.
Affected = colorectal cancer (CRC) cases;
Control (CTL) = healthy individuals;
P values reflect the significance of differences between cases and controls.
M = males,
F = females.

The T allele of SNP rs737865 is a protective factor in colorectal cancer—Genotyping of the C/T polymorphism of SNP rs737865 revealed that the CC genotype was associated with increased risk of CRC [p=1.19×10⁻² (Table 24), risk genotype GRR=1.39, OR 1.23; 95% C.I. 1.02-1.49 (Table 21)]. On the other hand, the T allele and the TT genotype were associated with control individuals (allele, p=1.78×10⁻³, genotype, p=8.59×10⁻³, Table 24), suggesting the T allele of SNP rs737865 is a protective factor against CRC.

TABLE 24
Allele and genotype frequencies of SNPs rs737865 in colorectal cancer case-control
study.

| | Affected | Control | Allele | CC genotype | TT genotype |
|---|---|---|---|---|---|
| Studied Group | (T Freq.) | (T Freq.) | P value | P value | P value |
| CRC/CTL | 938 (0.556) | 2951 (0.597) | 1.78 × 10⁻³ | 1.19 × 10⁻² | 8.59 × 10⁻³ |
| CRC M/CTL | 492 (0.558) | 2951 (0.597) | 2.18 × 10⁻² | 1.58 × 10⁻² | 1.38 × 10⁻¹ |
| CRC F/CTL | 442 (0.553) | 2951 (0.597) | 1.40 × 10⁻² | 1.78 × 10⁻¹ | 1.03 × 10⁻² |

Allele and genotype frequencies revealed by individual genotyping.
Affected = colorectal cancer (CRC) cases;
Control (CTL) = healthy individuals;
P values reflect the significance of differences between cases and controls.
M = males,
F = females.

The G-G haplotype of SNPs rs4680-165599 is a risk factor for colorectal cancer—To substantiate the association between colorectal cancer and SNPs in the COMT locus the frequencies of all possible haplotypes were compared between CRC cases and controls. A strong association was observed for the G-G haplotype of SNPs rs4680/rs165599 [p=4.63×10⁻⁶ (Table 25); OR 1.48; 95% C.I. 1.26-1.76] with CRC in females. Similar association was found between all CRC cases and controls [p=9.58×10⁻⁵ (Table 25); OR 1.23; 95% C.I. 1.11-1.38] suggesting that the G-G haplotype of SNPs rs4680-rs165599 is a risk factor for CRC. The population attributable risk of the G-G haplotype of SNPs rs4680-rs165599 was 11.5% in the general population and 24.4% in females. In addition, the genotype relative risk of the GG-GG double homozygote was 56% and 110% higher than of the protective double genotype in the general population and in females, respectively.

TABLE 25
Most significant haplotypes of SNPs rs4680, rs165599 associated with colorectal
cancer

| | | | | Combined | |
|---|---|---|---|---|---|
| | | Number | Number | Haplotype | Best Haplotype |
| SNP ID# | Studied Group | of affected | of control | P value | P value |
| rs4680/rs165599 | CRC/CTL | 931 | 4064 | 1.41 × 10⁻³ | 9.58 × 10⁻⁵ (GG) |
| rs4680/rs165599 | CRC M/CTL M | 490 | 3024 | 2.29 × 10⁻¹ | 6.61 × 10⁻² (AA) |
| rs4680/rs165599 | CRC F/CTL F | 437 | 1034 | 7.10 × 10⁻⁵ | 4.63 × 10⁻⁶ (GG) |

Haplotype analysis revealed by individual genotyping.
P values represent the significance of differences between cases and controls.
CRC = colorectal cancer cases;
CTL = healthy controls,
M = males,
F = females.

Altogether these results demonstrate a strong association of SNPs in the COMT locus with colorectal cancer. More particularly, the G allele of SNP rs4680, the G allele of SNP rs165599, the C allele of SNP rs737865 and the G-G haplotype of SNPs rs4680-rs165599 are risk factors for CRC. On the other hand, the T allele of SNP rs737865 is a protective factor against CRC. Therefore, these results suggest the use of SNPs rs4680, rs165599 and rs737865 and any SNP in LD with them for the diagnosis and prediction of an individual's risk of suffering form CRC. Furthermore, these markers can further contribute to disease treatment by suggesting drug candidates.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of determining a predisposition of a subject to develop schizophrenia, the method comprising determining a presence in a homozygous or heterozygous form, or an absence, of at least one genotype in the COMT locus or in neighboring loci, said neighboring loci are in linkage disequilibrium with said COMT locus, said at least one genotype having been associated with schizophrenia, thereby determining the predisposition of the subject of developing schizophrenia.

2. The method of claim 1, wherein said at least one genotype in said COMT locus is a guanine nucleotide—containing allele of SNP rs4680 set forth in SEQ ID NO:5.

3. The method of claim 1, wherein said at least one genotype in said COMT locus is a guanine nucleotide—containing allele of SNP rs165599 set forth in SEQ ID NO:9.

4. The method of claim 1, wherein said at least one genotype in said COMT locus is a cytosine nucleotide—containing allele of SNP rs737865 set forth in SEQ ID NO:1.

5. The method of claim 1, wherein said at least one genotype in said COMT locus is an allelic haplotype comprising at least two of:
a cytosine nucleotide—containing allele of SNP rs737865 set forth in SEQ ID NO:1;
a guanine nucleotide—containing allele of SNP rs4680 set forth in SEQ ID NO:5; and
a guanine nucleotide—containing allele of SNP rs165599 set forth in SEQ ID NO:9.

6. A method of determining a predisposition to develop schizophrenia in a female subject, the method comprising determining a presence in a homozygous or heterozygous form, or an absence, of at least one genotype in the COMT locus or in neighboring loci, said neighboring loci are in linkage disequilibrium with said COMT locus, said at least one genotype having been associated with schizophrenia in females in higher association than in males, thereby determining the predisposition of the female subject of developing schizophrenia.

7. The method of claim 6, wherein said at least one genotype in said COMT locus is a guanine nucleotide—containing allele of SNP rs165599 set forth in SEQ ID NO:9.

8. The method of claim 6, wherein said at least one genotype in said COMT locus is a cytosine nucleotide—containing allele of SNP rs737865 set forth in SEQ ID NO:1.

9. The method of claim 6, wherein said at least one genotype in said COMT locus is an allelic haplotype comprising at least two of:
a cytosine nucleotide—containing allele of SNP rs737865 set forth in SEQ ID NO:1;
a guanine nucleotide—containing allele of SNP rs4680 set forth in SEQ ID NO:5; and
a guanine nucleotide—containing allele of SNP rs165599 set forth in SEQ ID NO:9.

10-48. (canceled)

49. A kit for determining a predisposition of a subject to develop schizophrenia, the kit comprising a packaging material packaging at least one reagent, said at least one reagent for determining a presence in a homozygous or heterozygous form, or an absence, of at least one genotype in the COMT locus or in neighboring loci, said neighboring loci are in linkage disequilibrium with said COMT locus, said genotype having been associated with schizophrenia, the kit further comprising a notification in or on said packaging material, said notification identifying the kit for use in determining a predisposition of developing schizophrenia.

50. The kit of claim 49, wherein said notification also provides for instructions of using the kit in determining a predisposition of developing schizophrenia.

51. The kit of claim 50, wherein said at least one genotype in said COMT locus is a guanine nucleotide—and/or an adenosine nucleotide—containing allele of SNP rs4680 set forth in SEQ ID NO:5.

52. The kit of claim 50, wherein said at least one genotype in said COMT locus is a guanine nucleotide—and/or an adenosine nucleotide—containing allele of SNP rs165599 set forth in SEQ ID NO:9.

53. The kit of claim 50, wherein said at least one genotype in said COMT locus is a cytosine nucleotide—and/or a thymine nucleotide—containing allele of SNP rs737865 set forth in SEQ ID NO:1.

54. The kit of claim 50, wherein said at least one genotype in said COMT locus is an allelic haplotype comprising at least two of:
a cytosine nucleotide—and/or a thymine nucleotide—containing allele of SNP rs737865 set forth in SEQ ID NO:1;
a guanine nucleotide—and/or an adenosine nucleotide containing allele of SNP rs4680 set forth in SEQ ID NO:5; and
a guanine nucleotide—and/or an adenosine nucleotide containing allele of SNP rs165599 set forth in SEQ ID NO:9.

55. The kit of claim 50, wherein said at least one reagent is selected from the group consisting of at least one oligonucleotide, at least one antibody and a DNA chip.

56. The kit of claim 55, wherein said at least one oligonucleotide has a sequence selected differentially hybridizeable to at least one allele of an SNP selected from the group consisting of rs4680, rs165599 and rs737865, said at least one oligonucleotide can differentiate between polymorphs of said SNP via differential hybridization.

57. The kit of claim 55, wherein said at least one oligonucleotide has a sequence selected hybridizeable adjacent to an SNP selected from the group consisting of rs4680, rs165599 and rs737865, said at least one oligonucleotide can differentiate between polymorphs of said SNP via a differential template dependent primer extension reaction or a differential template dependent ligation reaction.

58. The kit of claim 55, wherein said at least one oligonucleotide has a sequence selected hybridizeable adjacent to an SNP selected from the group consisting of rs4680, rs165599 and rs737865, said at least one oligonucleotide can be used to amplify polymorphs of said SNP via an amplification reaction.

59. The kit of claim 55, wherein said at least one antibody is differentially interactable with at least one form of a COMT protein encoded by a COMT allele having an SNP rs4680, said at least one antibody can differentiate between polymorphs of said COMT protein via differential antibody interaction.

60. The kit of claim 55, wherein said DNA chip comprises attached thereto at least one oligonucleotide having a sequence selected differentially hybridizeable to at least one allele of an SNP selected from the group consisting of rs4680, rs165599 and rs737865, said at least one oligonucleotide can differentiate between polymorphs of said SNP via differential hybridization.

61. The kit of claim 49, wherein said at least one reagent is designed for performing a detection method selected from the group consisting of a signal amplification method, a direct detection method and detection of at least one sequence change.

62. The kit of claim 61, wherein said signal amplification method amplifies a molecule selected from the group consisting of a DNA molecule and an RNA molecule.

63. The kit of claim 61, wherein said signal amplification method is selected from the group consisting of PCR, LCR (LAR), Self-Sustained Synthetic Reaction (3SR/NASBA) and Q-Beta (Qβ) Replicase reaction.

64. The kit of claim 61, wherein said direct detection method is selected from the group consisting of a cycling probe reaction (CPR) and a branched DNA analysis.

65. The kit of claim 61, wherein said detection of at least one sequence change employs a method selected from the group consisting of restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis and Dideoxy fingerprinting (ddF).

66. The kit of claim 49, wherein said at least one reagent is designed for performing an immunological detection method for a COMT protein encoded by said COMT locus.

67. The kit of claim 66, wherein said immunological detection method is selected from the group consisting of a radio-immunoassay (RIA), an enzyme linked immunosorbent assay (ELISA), a western blot, an immunohistochemical analysis, and fluorescence activated cell sorting (FACS).

68-493. (canceled)


SEQUENCE LISTING (34 sequences)

SEQ ID NO: 1 - DNA, 343 bp, Homo sapiens
atcagcatggagccagctttttctcatgttgtcagtagttaccaagtaaaatctgttttttctgcattaactagtgtctcactgggctctgcagggcagctcctacggtccctcaggcttggagggtcactttaaacaataaaaagcaacaggacacaaaaayccctggctggaaaaatccaaaaagcaggtctgttagcagggcaggcccggagtgacttcccctttctctaacattcccacgtggtcctgcacaccacacatccccccgaccctggggagtcctgggccccttcctggtggagcagcctctctcttgcagggaaaggccctggggtacccc

SEQ ID NO: 2 - DNA, 21 bp, Artificial sequence
  Single strand DNA oligonucleotide
tagtgtctcactgggctctgc

SEQ ID NO: 3 - DNA, 21 bp, Artificial sequence
  Single strand DNA oligonucleotide
acctgctttttggatttttcc

SEQ ID NO: 4 - DNA, 14 bp, Artificial sequence
  Single strand DNA oligonucleotide
ttttccagccaggg

SEQ ID NO: 5 - DNA, 201 bp, Homo sapiens
gcgcatggcccgcctgctgtcaccaggggcgaggctcatcaccatcgagatcaaccccgactgtgccgccatcacccagcggatggtggatttcgctggcrtgaaggacaaggtgtgcatgcctgacccgttgtcagacctggaaaaagggccggctgtgggcagggagggcatgcgcactttgtcctccccaccaggtgt

SEQ ID NO: 6 - DNA, 21 bp, Artificial sequence
  Single strand DNA oligonucleotide
tcatcaccatcgagatcaacc

SEQ ID NO: 7 - DNA, 20 bp, Artificial sequence
  Single strand DNA oligonucleotide
ccctttttccaggtctgaca

SEQ ID NO: 8 - DNA, 15 bp, Artificial sequence
  Single strand DNA oligonucleotide
ggtggatttcgctgg

SEQ ID NO: 9 - DNA, 201 bp, Homo sapiens
ggcacgcctggcccactggcctcccagccacagtggtgcagaggtcagccctcctgcagctaggccaggggcacctgttagccccatggggacgactgccrgcctgggaaacgaagaggagtcagccagcattcacacctttctgaccaagcaggcgctggggacaggtggaccccgcagcagcaccagcccctctgggcc

SEQ ID NO: 10 - DNA, 20 bp, Artificial sequence
  Single strand DNA oligonucleotide
cacagtggtgcagaggtcag

SEQ ID NO: 11 - DNA, 21 bp, Artificial sequence
  Single strand DNA oligonucleotide
ctggctgactcctcttcgttt

SEQ ID NO: 12 - DNA, 14 bp, Artificial sequence
  Single strand DNA oligonucleotide
ttcgtttcccaggc

SEQ ID NO: 13 - DNA, 201 bp, Homo sapiens
acccgagggcaccagagggcacgagaaggctggctccctggcgctgacacgtcaggcaactgaggcacaaggctggcatttctgaaccttgcccctctgcraacacaagggggcgatggtggcactccaagcaaaggggcgtgtgggtgctgcaggaggagcacagagcactggcgcccctcccctcccgccctgcagatg

SEQ ID NO: 14 - DNA, 21 bp, Artificial sequence
  Single strand DNA oligonucleotide
atttctgaaccttgcccctct

SEQ ID NO: 15 - DNA, 20 bp, Artificial sequence
  Single strand DNA oligonucleotide
cagtgctctgtgctcctcct

SEQ ID NO: 16 - DNA, 13 bp, Artificial sequence
  Single strand DNA oligonucleotide
tcgcccccttgtg

SEQ ID NO: 17 - DNA, 201 bp, Homo sapiens
ggggctggggcctgtgccttatcggctggaacgagttcatcctgcagcccatccacaacctgctcatgggtgacaccaaggagcagcgcatcctgaaccaygtgctgcagcatgcggagcccgggaacgcacagagcgtgctggaggccattgacacctactgcgagcagaaggagtgggccatgaacgtgggcgacaaga

SEQ ID NO: 18 - DNA, 19 bp, Artificial sequence
  Single strand DNA oligonucleotide
catgggtgacaccaaggag

SEQ ID NO: 19 - DNA, 21 bp, Artificial sequence
  Single strand DNA oligonucleotide
ctgctcgcagtaggtgtcaat

SEQ ID NO: 20 - DNA, 14 bp, Artificial sequence
  Single strand DNA oligonucleotide
cgcatcctgaacca

SEQ ID NO: 21 - DNA, 401 bp, Homo sapiens
  misc_feature (201)..(201): missing nucleotide or G
taaagaagtcatgattgagtcttaaaaaagaacaatccagtgttgcagttcagagaggttagcatgtcagggcgcaggcctcggccgaggtgtgctttgcatttaggacacagcccggagccgcagaaggtcagcaggagcacgtctggcaccttcagtaccaggctgggtgagagagcccgagaggggggccgggggggncagtcagggccctgcttcgctgcctgggcccttgtagatggccttctccaggccgtccaccacctccctgtattccaggaacgattggtagtgtgtgcactcaaagcagctgctcccgcgcacgtgtgctaggaagtctggcgcacctgggcagatcacgttgtcagccagtagcactgtccccttccgcagcaggccac

SEQ ID NO: 22 - DNA, 20 bp, Artificial sequence
  Single strand DNA oligonucleotide
gttctctgggcacctctgac

SEQ ID NO: 23 - DNA, 21 bp, Artificial sequence
  Single strand DNA oligonucleotide
ctggctgactcctcttcgttt

SEQ ID NO: 24 - DNA, 133 bp, Homo sapiens
ttgggaagtctggcyagtggggccggtgcctggtgacctcgggaggtgggatatcatcatcttcagaactgtagttgttactgggataccagctctgggagaccacaggtgcagtcagcacagcaggacctta

SEQ ID NO: 25 - DNA, 20 bp, Artificial sequence
  Single strand DNA oligonucleotide
gctattgccgtgtctggact

SEQ ID NO: 26 - DNA, 20 bp, Artificial sequence
  Single strand DNA oligonucleotide
ctgactgcacctgtggtctc

SEQ ID NO: 27 - DNA, 101 bp, Homo sapiens
attattgtcctgatttagttaactgttcttcaggggctccaggaggacgartgtgtatcctcccattgctctgtgcagcctctaacctctagagtctaggg

SEQ ID NO: 28 - DNA, 101 bp, Homo sapiens
tttggaatttggctattgccgtgtctggactgtgagtatgggaaggggaarcttttctgtctgttgtccccactaccgcccctcacatccgtgattctgaa

SEQ ID NO: 29 - protein, 271 aa, Homo sapiens
  misc_feature (158)..(158): Valine or Methionine
MPEAPPLLLAAVLLGLVLLVVLLLLLRHWGWGLCLIGWNEFILQPIHNLLMGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGXKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP

SEQ ID NO: 30 - protein, 221 aa, Homo sapiens
  misc_feature (108)..(108): Valine or Methionine
MGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGXKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP

SEQ ID NO: 31 - protein, 962 aa, Homo sapiens
MEDCNVHSAASILASVKEQEARFERLTRALEQERRHVALQLERAQQPGMVSGGMGSGQPLPMAWQQLVLQEQSPGSQASLATMPEAPDVLEETVTVEEDPGTPTSHVSIVTSEDGTTRRTETKVTKTVKTVTTRTVRQVPVGPDGLPLLDGGPPLGPFADGALDRHFLLRGGGPVATLSRAYLSSGGGFPEGPEPRDSPSYGSLSRGLGMRPPRAGPLGPGPGDGCFTLPGHREAFPVGPEPGPPGGRSLPERFQAEPYGLEDDTRSLAADDEGGPELEPDYGTATRRRPECGRGLHTRAYEDTADDGGELADERPAFPMVTAPLAQPERGSMGSLDRLVRRSPSVDSARKEPRWRDPELPEVLAMLRHPVDPVKANAAAYLQHLCFENEGVKRRVRQLRGLPLLVALLDHPRAEVRRRACGALRNLSYGRDTDNKAAIRDCGGVPALVRLLRAARDNEVRELVTGTLWNLSSYEPLKMVIIDHGLQTLTHEVIVPHSGWEREPNEDSKPRDAEWTTVFKNTSGCLRNVSSDGAEARRRLRECEGLVDALLHALQSAVGRKDTDNKSVENCVCIMRNLSYHVHKEVPGADRYQEAEPGPLGSAVGSQRRRRDDASCFGGKKAKEEWFHQGKKDGEMDRNFDTLDLPKRTEAAKGFELLYQPEVVRLYLSLLTESRNFNTLEAAAGALQNLSAGNWMWATYIRATVRKERGLPVLVELLQSETDKVVRAVAIALRNLSLDRRNKDLIGSYAMAELVRNVRNAQAPPRPGACLEEDTVVAVLNTIHEIVSDSLDNARSLLQARGVPALVALVASSQSVREAKAASHVLQTVWSYKELRGTLQKDGWTKARFQSAAATAKGPKGALSPGGFDDSTLPLVDKSLEGEKTGSRDVIPMDALGPDGYSTVDRRERRPRGASSAGEASEKEPLKLDPSRKAPPPGPSRPAVRLVDAVGDAKPQPVDSWV

SEQ ID NO: 32 - protein, 522 aa, Homo sapiens [sequence omitted]

SEQ ID NO: 33 - protein, 426 aa, Homo sapiens
MHQAALLGGLIQDAPNYGWEVAQPVPHDWRKMAEAVQNHVKSLNWGHRVQLQDRKVKYFNIKASFVDEHTVCGVAKGGKEILLSADHIIIATGGRPRYPTHIEGALEYGITSDDIFWLKESPGKTLVVGASYVALECAGFLTGIGLDTTIMMRSIPLRGFDQQMSSMVIEHMASHGTRFLRGCAPSRVRRLPDGQLQVTWEDSTTGKEDTGTFDTVLWAIGRVPDTRSLNLEKAGVDTSPDTQKILVDSREATSVPHIYAIGDVVEGRPELTPIAIMAGRLLVQRLFGGSSDLMDYDNVPTTVFTPLEYGCVGLSEEEAVARHGQEHVEVYHAHYKPLEFTVAGRDASQCYVKMVCLREPPQLVLGLHFLGPNAGEVTQGFALGIKCGASYAQVMRTVGIHPTCSEEVVKLRISKRSGLDPTVTGC

SEQ ID NO: 34 - protein, 492 aa, Homo sapiens
MEDQAGQRDYDLLVVGGGSGGLACAKEAAQLGRKVAVVDYVEPSPQGTRWGLGGTCVNVGCIPKKLMHQAALLGGLIQDAPNYGWEVAQPVPHDWRKMAEAVQNHVKSLNWGHRVQLQDRKVKYFNIKASFVDEHTVCGVAKGGKEILLSADHIIIATGGRPRYPTHIEGALEYGITSDDIFWLKESPGKTLVVGASYVALECAGFLTGIGLDTTIMMRSIPLRGFDQQMSSMVIEHMASHGTRFLRGCAPSRVRRLPDGQLQVTWEDSTTGKEDTGTFDTVLWAIGRVPDTRSLNLEKAGVDTSPDTQKILVDSREATSVPHIYAIGDVVEGRPELTPIAIMAGRLLVQRLFGGSSDLMDYDNVPTTVFTPLEYGCVGLSEEEAVARHGQEHVEVYHAHYKPLEFTVAGRDASQCYVKMVCLREPPQLVLGLHFLGPNAGEVTQGFALGIKCGASYAQVMRTVGIHPTCSEEVVKLRISKRSGLDPTVTGC